(19) United States
(12) Patent Application Publication
(10) Pub. No.: US 2023/0148956 A1
(43) Pub. Date: May 18, 2023

(54) APPARATUS FOR HEALTH MONITORING

(71) Applicant: Eisai R&D Management Co., Ltd., Bunkyo-ku, Tokyo (JP)

(72) Inventors: Rajeev NARAYANAN, Woodcliff Lake, NJ (US); Tushar D. PATEL, Hillsborough, NJ (US); Alexander SCOTT, Montclair, NJ (US); Mitsuo KOSAKA, Edgewater, NJ (US); David LEVIN, Highland Mills, NY (US)

(73) Assignee: Eisai R&D Management Co., Ltd., Bunkyo-ku, Tokyo (JP)

(21) Appl. No.: 17/913,635

(22) PCT Filed: Mar. 25, 2021
(86) PCT No.: PCT/US2021/024057

§ 371 Date: Sep. 22, 2022

Related U.S. Application Data
(60) Provisional application No. 63/000,083, filed on Mar. 26, 2020.
(60) Provisional application No. 63/007,626, filed on Apr. 9, 2020.

(51) Int. Cl. A61B 5/00; A61B 5/11

(52) U.S. Cl. CPC A61B 5/4848; A61B 5/6802; A61B 5/486; A61B 5/1101; A61B 5/4812; A61B 5/4815; A61B 5/4809; A61B 2562/046; A61B 2562/0219

(57) ABSTRACT

An apparatus for monitoring a user's health and/or sleep and/or the efficacy of a treatment (e.g. a sleep treatment or other type of health treatment) can include use of a wearable electronic device. The device can include an array of sensors for collecting user data. The user data can be used by the device to evaluate criteria related to the user's health to monitor efficacy of a treatment. In addition, or alternatively, the collected data can be transmitted to a central server and/or input/output device for evaluating different criteria for monitoring the user's health and/or sleep as well as the efficacy of a treatment being provided to the user.

### CROSS-REFERENCE TO RELATED APPLICATIONS

The present application claims priority to U.S. Provisional Patent Application No. 63/000,083, filed on Mar. 26, 2020. The present application also claims priority to U.S. Provisional Patent Application No. 63/007,626, filed on Apr. 9, 2020.

### FIELD

The present innovation relates to electronic devices and communication systems that can be configured to facilitate the monitoring of a patient's health, which can include, for example, the patient's sleep and/or other health parameters. In some embodiments, the device can also be (or alternatively be) configured to evaluate the efficacy of a treatment the patient is utilizing to improve a health parameter (e.g. a sleep treatment for a patient who has been diagnosed as having a sleep related health issue, etc.). This present innovation also relates methods of making and using embodiments of the device and system, methods of evaluating a health improvement treatment and methods of facilitating the diagnosis of a health issue.

### BACKGROUND

Connected wearable devices such as a watch can be configured to communicate data to a smart phone or smart speaker about a user's health. Examples of these types of devices are disclosed in U.S. Pat. Nos. 8,075,499, 8,945,017, 9,582,034, 9,743,848, 10,146,196, 10,506,944, as well as U.S. Patent Application Publication Nos. 2017/0358942 and 2015/0290419 and U.S. Design Pat. Nos. D760,395 and D867,599. Such biometric data can include heart beat sensor data, core body temperature, oxygen saturation level (SPO₂), and activity estimation data. We have determined that such devices and systems often are unable to take into account user specific data. For instance, such devices and systems often are unable to take into consideration a particular treatment the user is being administered to help the person address a health issue.

### SUMMARY

We have determined that an apparatus can be designed to help address sleep related issues a person may have. For instance, sleep can be comorbid to other diseases such as diabetes, dementia, ADHD, Epilepsy, etc. Embodiment of the apparatus can be configured to integrate pathological and biological data of a person with the biometric, health record (e.g. A1C, Blood pressure, Blood-urea ratio, etc.), and clinical data to predict their health state in real-time.

Embodiments of the apparatus can be configured as a wearable device or a system that utilizes a wearable device that can communicatively connect to at least one other device (e.g. a server and/or an input/output device such as a smart phone, laptop computer, tablet, or other type of computer device or communication device) to facilitate the monitoring of a patient and/or the efficacy of a treatment the patient is being administered. Embodiments of methods for making and using such apparatuses are also provided. Embodiments of the apparatuses can also be utilized in methods of evaluating a sleep treatment and methods of facilitating the diagnosis of a sleep related health issue. Embodiments of the apparatuses can also be utilized in methods of evaluating a treatment for a neurological disorder and neurodegenerative diseases associated with tremor and/or for which tremor is a symptom. A tremor can be associated with a number of neurological disorders, including, for example, multiple sclerosis, stroke, and traumatic brain injury, and neurodegenerative diseases that affect parts of the brain, including, for example, Parkinson's disease, attention deficit hyperactivity disorder (ADHD), attention deficit disorder (ADD), dementia, or Alzheimer's disease. Embodiments of the apparatuses can also be utilized in methods of evaluating tremors which result from the use of certain medicines (for example, asthma medication, amphetamines, caffeine, corticosteroids, and drugs used for certain psychiatric and neurological disorders), alcohol abuse or alcohol withdrawal, overactive thyroid, liver failure, kidney failure, anxiety or panic. A number of different forms of tremor exist and may be detected by embodiments of the apparatuses disclosed herein, including, for example, essential tremor (also known as benign essential tremor or familial tremor), dystonic tremor, cerebellar tremor (e.g., tremors caused by damage to the cerebellum and its pathways to other brain regions resulting from a stroke or tumor, or caused by disease (such as multiple sclerosis or an inherited degenerative disorder, e.g., ataxia and Fragile X syndrome) or resulting from chronic damage to the cerebellum due to alcoholism), psychogenic tremor (also called functional, and includes tremors resulting from stress or a psychiatric disorder such as depression or post-traumatic stress disorder (PTSD)), enhanced physiological tremor (e.g., tremors resulting from certain drugs, alcohol withdrawal, or medical conditions including an overactive thyroid and hypoglycemia, Parkinsonian tremor, and orthostatic tremor.

In a first aspect, an apparatus for health monitoring can include one or more of: (i) a wearable device comprising a processor, a non-transitory computer readable medium connected to the processor; and a sensor array connected to the processor and/or the non-transitory computer readable medium, (ii) a server communicatively connectable to the wearable deice to receive sensor data from the wearable device, and (iii) an input/output device communicatively connectable to the wearable device to receive sensor data from the wearable device. In some embodiments, the apparatus can include the wearable device and the input/output device. In other embodiments, the apparatus can include the input/output device and the server. In yet other embodiments, the apparatus can include the wearable device, the server, and the input/output device. In yet other embodiments, the apparatus can include only one of the wearable device, the server, and the input/output device.

In a second aspect, the apparatus for health monitoring can include at least the wearable device. The wearable device can be configured to obtain the sensor data via the sensor array when a user wears the wearable device, analyze the sensor data to track a condition of the user, and generate output to help the user improve the condition or maintain the condition. In the second aspect of the apparatus, the apparatus may only include the wearable device, may include the wearable device and the input/output device, the wearable device and the server, or may include the server, the input/output device, and the wearable device.

In a third aspect, the wearable device of the apparatus for health monitoring can be configured to generate the output to suggest the change to the user to help the user improve the condition based on the sensor data and subjective data provided by the user. The output can include visual output via a graphical user interface (GUI), text, graphical representation of data, audible output emitted from at least one speaker, tactile output (e.g. a vibration output from a vibration mechanism), or other type of output. The output that is generated can be output from the wearable device or can be communicated to another device for being emitted (e.g. to an input/output device for emission by a speaker or screen of the input/output device, etc.).

In a fourth aspect, the wearable device of the apparatus for health monitoring can be configured to obtain the sensor data via the sensor array when a user wears the wearable device, analyze the sensor data to track a condition of the user and determine a baseline for the condition. The wearable device can also be configured to respond to a first input indicating that a drug at a first dosage is being taken by the user by comparing the baseline for the condition with the sensor data obtained after the receipt of the first input to determine whether the condition has improved.

In a fifth aspect, the wearable device of the apparatus for health monitoring can be configured to respond to a determination that the condition has not improved within a pre-specified improvement time period after receipt of the first input by suggesting a change to the user for adjusting a frequency of taking the drug and/or adjusting a dosage of the drug to a second dosage that differs from the first dosage. The second dosage may be a higher dosage than the first dosage or a lower dosage than the first dosage. The frequency adjustment for the drug can be taking the drug more frequently or less frequently. For instance, frequency adjustment may occur by the same dosage of the drug or a different dosage of the drug being taken ever 2-4 hours instead of once a day or being taken 4-6 hours instead of being taken every 2-4 hours, or being taken every 6-12 hours instead of once a day, etc. Other types of frequency adjustments can also be provided.

In a sixth aspect, the wearable device of the apparatus for health monitoring can be configured to respond to a second input indicating that a change to the drug being taken by the user has occurred by comparing the baseline for the condition with the sensor data obtained after the receipt of the second input to determine whether the condition has improved. The change to the drug can include a change in dosage of the drug, a change in the frequency at which the drug is being taken, or both a change in dosage and a change in frequency.

In a seventh aspect, the wearable device of the apparatus for health monitoring can be configured to respond to a determination that the condition has not improved within a pre-specified improvement time period after receipt of the second input by suggesting a change to the user for adjusting a frequency of taking the drug and/or adjusting a dosage of the drug to a third dosage that differs from the first dosage. The third dosage may be a higher dosage than the first dosage or a lower dosage than the first dosage. The third dosage may be a higher dosage than the second dosage or a lower dosage than the second dosage as well. The frequency adjustment for the drug can be taking the drug more frequently or less frequently. For instance, frequency adjustment may occur by the same dosage of the drug or a different dosage of the drug being taken ever 2-4 hours instead of once a day or being taken 4-6 hours instead of being taken every 2-4 hours, or being taken every 6-12 hours instead of once a day, etc. Other types of frequency adjustments can also be provided.

In an eighth aspect, the apparatus for monitoring health can include at least he server. The server can be configured to analyze the sensor data to track a condition of the user and generate output to suggest a change to help the user improve the condition or maintain the condition for output via the wearable device and/or the input/output device to which the server is communicatively connectable. In the eighth aspect of the apparatus, the apparatus may only include the server, may include the server and the input/output device, the server and the wearable device, or may include the server, the input/output device, and the wearable device.

In a ninth aspect, the apparatus for health monitoring can be configured so that the server is configured to generate the output to suggest the change to the user to help the user improve the condition based on the sensor data and subjective data provided by the user. The subjective data provided by the user can be provided via at least one input device of the wearable device and/or the input/output device. Such input can be provided via a microphone, pointer device, touch screen, or other type of input device, for example. The user subjective data can be provided to the server via a communication connection the server has with the wearable device or the input/output device (e.g. an application programming interface connection provided via at least one network, via an internet connection, via an intranet communication connection, etc.).

In a tenth aspect, the server of the apparatus for health monitoring can be configured to evaluate the sensor data to track a condition of the user and determine a baseline for the condition. The server can also be configured to respond to a first input indicating that a drug at a first dosage is being taken by the user by comparing the baseline for the condition with the sensor data obtained after the receipt of the first input to determine whether the condition has improved. In some embodiments, the first input can be provided by the user via at least one input device of the wearable device and/or the input/output device. Such input can be provided via a microphone, pointer device, touch screen, or other type of input device, for example. The first input can be provided to the server via a communication connection the server has with the wearable device or the input/output device (e.g. an application programming interface connection provided via at least one network, via an internet connection, via an intranet communication connection, etc.).

In an eleventh aspect, server of the apparatus for health monitoring can be configured to respond to a determination that the condition has not improved within a pre-specified improvement time period after receipt of the first input by suggesting a change to the user for adjusting a frequency of taking the drug and/or adjusting a dosage of the drug to a second dosage that differs from the first dosage. The second dosage may be a higher dosage than the first dosage or a lower dosage than the first dosage. The frequency adjustment for the drug can be taking the drug more frequently or less frequently. For instance, frequency adjustment may occur by the same dosage of the drug or a different dosage of the drug being taken ever 2-4 hours instead of once a day or being taken 4-6 hours instead of being taken every 2-4 hours, or being taken every 6-12 hours instead of once a day, etc. Other types of frequency adjustments can also be provided.

In a twelfth aspect, the server of the apparatus for monitoring health can be configured to respond to a second input indicating that a change to the drug being taken by the user has occurred by comparing the baseline for the condition with the sensor data obtained after the receipt of the second input to determine whether the condition has improved. In some embodiments, the second input can be provided by the user via at least one input device of the wearable device and/or the input/output device. Such input can be provided via a microphone, pointer device, touch screen, or other type of input device, for example. The second input can be provided to the server via a communication connection the server has with the wearable device or the input/output device (e.g. an application programming interface connection provided via at least one network, via an internet connection, via an intranet communication connection, etc.).

In a thirteenth aspect, the server of the apparatus for health monitoring can be configured to respond to a determination that the condition has not improved within a pre-specified improvement time period after receipt of the second input by suggesting a change to the user for adjusting a frequency of taking the drug and/or adjusting a dosage of the drug to a third dosage that differs from the first dosage. The change to the drug can include a change in dosage of the drug, a change in the frequency at which the drug is being taken, or both a change in dosage and a change in frequency. For instance, the third dosage may be a higher dosage than the first dosage or a lower dosage than the first dosage. The third dosage may be a higher dosage than the second dosage or a lower dosage than the second dosage as well. The frequency adjustment for the drug can be taking the drug more frequently or less frequently. For instance, frequency adjustment may occur by the same dosage of the drug or a different dosage of the drug being taken ever 2-4 hours instead of once a day or being taken 4-6 hours instead of being taken every 2-4 hours, or being taken every 6-12 hours instead of once a day, etc. Other types of frequency adjustments can also be provided.

In a fourteenth aspect, the apparatus for health monitoring can include the input/output device. The input/output device can be configured to analyze the sensor data to track a condition of the user and generate output to suggest a change to the user to help the user improve the condition for output to the user. Embodiments of the fourteenth aspect of the apparatus can only include the input/output device or can include the input/output device in combination with the server, input/output device in combination with the wearable device or can include the input/output device in combination with the server and the wearable device.

In a fifteenth aspect, the input/output device of the apparatus for health monitoring can be configured to generate the output to suggest the change to help the user improve the condition or maintain the condition based on the sensor data and subjective data provided by the user. The output that is generated can include visual output via a graphical user interface (GUI), text, graphical representation of data, audible output emitted from at least one speaker, tactile output (e.g. a vibration output from a vibration mechanism), or other type of output. The output that is generated can be output from the input/output device and/or can be communicated to another device for being emitted (e.g. to a wearable device for emission by a speaker, screen or vibration mechanism of the wearable device, etc.).

In a sixteenth aspect, the input/output device of the apparatus for health monitoring can be configured to evaluate the sensor data to track a condition of the user and determine a baseline for the condition. The input/output device can also be configured to respond to a first input indicating that a drug at a first dosage is being taken by the user by comparing the baseline for the condition with the sensor data obtained after the receipt of the first input to determine whether the condition has improved. The first input can be input a user provided via an input device of the input/output device (e.g. a pointer device, touch screen display, button, keypad, etc.).

In a seventeenth aspect, the input/output device of the apparatus for health monitoring can be configured to respond to a determination that the condition has not improved within a pre-specified improvement time period after receipt of the first input by suggesting a change to the user for adjusting a frequency of taking the drug and/or adjusting a dosage of the drug to a second dosage that differs from the first dosage. The second dosage may be a higher dosage than the first dosage or a lower dosage than the first dosage. The frequency adjustment for the drug can be taking the drug more frequently or less frequently. For instance, frequency adjustment may occur by the same dosage of the drug or a different dosage of the drug being taken ever 2-4 hours instead of once a day or being taken 4-6 hours instead of being taken every 2-4 hours, or being taken every 6-12 hours instead of once a day, etc. Other types of frequency adjustments can also be provided.

In an eighteenth aspect, the input/output device of the apparatus for health monitoring can be configured to respond to a second input indicating that a change to the drug being taken by the user has occurred by comparing the baseline for the condition with the sensor data obtained after the receipt of the second input to determine whether the condition has improved. The second input can be provided by a user via an input device of the input/output device in some embodiments.

In a nineteenth aspect, the input/output device of the apparatus for health monitoring can be configured to respond to a determination that the condition has not improved within a pre-specified improvement time period after receipt of the second input by suggesting a change to the user for adjusting a frequency of taking the drug and/or adjusting a dosage of the drug to a third dosage that differs from the first dosage. The third dosage may be a higher dosage than the first dosage or a lower dosage than the first dosage. The third dosage may be a higher dosage than the second dosage or a lower dosage than the second dosage as well. The frequency adjustment for the drug can be taking the drug more frequently or less frequently. For instance, frequency adjustment may occur by the same dosage of the drug or a different dosage of the drug being taken ever 2-4 hours instead of once a day or being taken 4-6 hours instead of being taken every 2-4 hours, or being taken every 6-12 hours instead of once a day, etc. Other types of frequency adjustments can also be provided.

In a twentieth aspect, the apparatus for monitoring health can be configured so that the condition that is monitored and/or evaluated is a tremor condition, epilepsy, a sleep condition, an Alzheimer's disease condition, a neurological disorder, a neurodegenerative disease associated with a tremor or for which the tremor is a symptom, multiple sclerosis, stroke, traumatic brain injury, Parkinson's disease, ADHD, dementia, Alzheimer's disease, the condition is a result of use of a medicine, the condition is a result of alcohol abuse, the condition is a withdrawal of a drug, the condition is a thyroid condition, the condition is an overactive thyroid, the condition is a liver condition, the condition is liver failure, the condition is kidney failure, the condition is anxiety or the condition is panic. Embodiments of the apparatus can include only the input/output device, only the wearable device, only the server, or combinations of these devices.

In a twenty-first aspect, the wearable device can be configured to evaluate the sensor data to track sleep of the user to determine a baseline level of sleep for the user. In a twenty-second aspect, the wearable device can be configured to monitor sleep of the user when the user wears the wearable device and determine one or more time durations at which the user was in a light sleep state during the sleep, a deep sleep state during the sleep, and/or a rapid eye movement (REM) sleep state during the sleep. In a twenty-third aspect, the wearable device can be configured to automatically determine when the user has woken up to stop monitoring of the sleep of the user. In a twenty-fourth aspect, the wearable device can be configured to automatically determine when the user has begun attempting to go to sleep to start monitoring of the sleep of the user. Each of the twenty-first through twenty-fourth aspects can be included in any of the other aspects discussed herein.

In a twenty-fifth aspect, the wearable device can be configured to calculate (i) a total time slept during a monitoring of the sleep, (ii) a time of sleep onset indicating a time it took the user to fall asleep after being detected as attempting to go to sleep, and/or (iii) a sleep efficiency indicating an amount of time the user was asleep during the total time the sleep of the user was monitored. In some embodiments, only one of these parameters (i)-(iii) can be calculated, in other embodiments, only two of these parameters (i)-(iii) can be calculated and in yet other embodiments all three of parameters (i)-(iii) can be calculated.

In a twenty-sixth aspect, the wearable device can be configured to determine (i) an amount of time during the monitored sleep that the user was in the light sleep state, (ii) an amount of time during the monitored sleep that the user was in the deep sleep state, and/or (iii) an amount of time during the monitored sleep that the user was in the REM sleep state. In some embodiments, only one of these parameters (i)-(iii) can be calculated, in other embodiments, only two of these parameters (i)-(iii) can be calculated and in yet other embodiments all three of parameters (i)-(iii) can be calculated.

In a twenty-seventh aspect, the wearable device can be configured to determine a sleep score for the user based on the sensor data obtained from monitoring of the sleep of the user. In a twenty-eighth aspect, the sleep score can be determined in accordance with a sleep score formula:

Sleep Score=LS*LS+W_{DS}*DS+W_{RS}*RS+W_{BMsleep}*BMₛₗₑₑₚ,W_{ENV}*ENV+W_{Td}*Td+W_{To}*To+W_{Te}*Te;

wherein:
LS is a value for an amount of time the user was determined to be in a light sleep state;
DS is a value for an amount of time the user was determined to be in a deep sleep state;
RS is value for an amount of time the user was determined to be in a REM sleep state;
Td is a value for duration of total sleep time of the user;
To is a value for a determined sleep onset for the sleep;
Te is a value for the determined sleep efficiency for the sleep;
BMₛₗₑₑₚ is a value for the detected body movement of the user during the sleep;
ENV is a value for the detected environment during the sleep;
W_{LS} is the weight for LS;
W_{DS} is the weight for DS;
W_{RS} is the weight for RS;
W_{BMsleep} is the weight for BMₛₗₑₑₚ,
W_{ENV} is the weight for ENV;
W_{Td} is the weight for Td;
W_{To} is the weight for To; and
W_{Te} is the weight for Te.

Each of the twenty-fifth through twenty-eighth aspects can be included in any of the other aspects discussed herein.

In a twenty-ninth aspect, the server can be configured to evaluate the sensor data to track sleep of the user to determine a baseline level of sleep for the user. In a thirtieth aspect, the server can be configured to monitor sleep of the user based on the sensor data and determine one or more time durations at which the user was in a light sleep state during the sleep, a deep sleep state during the sleep, and/or a rapid eye movement (REM) sleep state during the sleep. In a thirty-first aspect, the server can be configured to automatically determine when the user has woken up to define an end of the sleep of the user for determining a total duration of sleep of the user. In a thirty-second aspect, the server can be configured to automatically determine when the user began attempting to go to sleep and when the user first fell asleep during the sleep for determining a duration of time for sleep onset of the sleep. Each of the twenty-ninth aspect through the thirty-second aspect can be utilized in any of the other aspects discussed herein.

In a thirty-third aspect, the server can be configured to calculate (i) a total time slept during the sleep, (ii) a time of sleep onset indicating a time it took the user to fall asleep after being detected as attempting to go to sleep, and/or (iii) a sleep efficiency indicating an amount of time the user was asleep during the total time the sleep based on the sensor data. In some embodiments, only one of the parameters (i)-(iii) may be calculated, in other embodiments only two of the parameters (i)-(iii) can be calculated, and in yet other embodiments at least all three of the parameters (i)-(iii) can be calculated.

In a thirty-fourth aspect, the server can be configured to determine (i) an amount of time during the monitored sleep that the user was in the light sleep state, (ii) an amount of time during the monitored sleep that the user was in the deep sleep state, and/or (iii) an amount of time during the monitored sleep that the user was in the REM sleep state based on the sensor data. In some embodiments, only one of the parameters (i)-(iii) can be determined. In other embodiments only two of the parameters (i)-(iii) can be determined. In yet other embodiments at least all three of the parameters (i)-(iii) can be determined.

In a thirty-fifth aspect, the server can be configured to determine a sleep score for the user based on the sensor data. In a thirty-sixth aspect, the sleep score can be determined in accordance with a sleep score formula:

Sleep Score=W_{LS}*LS+W_{DS}*DS+W_{RS}*RS+W_{BMsleep}*BMₛₗₑₑₚ,+W_{ENV}*ENV+W_{Td}*Td+W_{To}*To+W_{Te}*Te;

wherein:
LS is a value for an amount of time the user was determined to be in a light sleep state;
DS is a value for an amount of time the user was determined to be in a deep sleep state;
RS is value for an amount of time the user was determined to be in a REM sleep state;
Td is a value for duration of total sleep time of the user;
To is a value for a determined sleep onset for the sleep;
Te is a value for the determined sleep efficiency for the sleep;
BMₛₗₑₑₚ is a value for the detected body movement of the user during the sleep;
ENV is a value for the detected environment during the sleep;
W_{LS} is the weight for LS;
W_{DS} is the weight for DS;
W_{RS} is the weight for RS;
W_{BMsleep} is the weight for BMₛₗₑₑₚ,
W_{ENV} is the weight for ENV;
W_{Td} is the weight for Td;
W_{To} is the weight for To; and
W_{Te} is the weight for Te.

Each of the thirty-third through thirty-sixth aspects can be utilized in any of the other aspects discussed herein.

In a thirty-seventh aspect, the input/output device can be configured to evaluate the sensor data to track sleep of the user to determine a baseline level of sleep for the user. In a thirty-eight aspect, the input/output device can be configured to monitor sleep of the user based on the sensor data and determine one or more time durations at which the user was in a light sleep state during the sleep, a deep sleep state during the sleep, and/or a rapid eye movement (REM) sleep state during the sleep. In a thirty-ninth aspect, the the input/output device can be configured to automatically determine when the user has woken up to define an end of the sleep of the user for determining a total duration of sleep of the user. In a fortieth aspect, the input/output device can be configured to automatically determine when the user began attempting to go to sleep and when the user first fell asleep during the sleep for determining a duration of time for sleep onset of the sleep. Each of the thirty-seventh through fortieth aspects can be utilized in any of the other aspects discussed herein.

In a forty-first aspect, the input/output device can be configured to calculate (i) a total time slept during the sleep, (ii) a time of sleep onset indicating a time it took the user to fall asleep after being detected as attempting to go to sleep, and/or (iii) a sleep efficiency indicating an amount of time the user was asleep during the total time the sleep based on the sensor data. In some embodiments, only one of the parameters (i)-(iii) can be calculated, in other embodiments only two of the parameters (i)-(iii) can be calculated, and in yet other embodiments at least all three of the parameters (i)-(iii) can be calculated.

In a forty-second aspect, the input/output device can be configured to determine (i) an amount of time during the monitored sleep that the user was in the light sleep state, (ii) an amount of time during the monitored sleep that the user was in the deep sleep state, and/or (iii) an amount of time during the monitored sleep that the user was in the REM sleep state based on the sensor data. In some embodiments, only one of the parameters (i)-(iii) may be determined, in other embodiments only two of the parameters (i)-(iii) can be determined, and in yet other embodiments at least all three of the parameters (i)-(iii) can be determined.

In a forty-third aspect, the server can be configured to determine a sleep score for the user based on the sensor data. In a forty-fourth aspect, the sleep score can be determined in accordance with a sleep score formula:

Sleep Score=W_{LS}*LS+W_{DS}*DS+W_{RS}*RS+W_{BMsleep}*BMₛₗₑₑₚ,+W_{ENV}*ENV+W_{Td}*Td+W_{To}*To+W_{Te}*Te;

wherein:
LS is a value for an amount of time the user was determined to be in a light sleep state;
DS is a value for an amount of time the user was determined to be in a deep sleep state;
RS is value for an amount of time the user was determined to be in a REM sleep state;
Td is a value for duration of total sleep time of the user;
To is a value for a determined sleep onset for the sleep;
Te is a value for the determined sleep efficiency for the sleep;
BMₛₗₑₑₚ is a value for the detected body movement of the user during the sleep;
ENV is a value for the detected environment during the sleep;
W_{LS} is the weight for LS;
W_{DS} is the weight for DS;
W_{RS} is the weight for RS;
W_{BMsleep} is M the weight for BMₛₗₑₑₚ,
W_{ENV} is the weight for ENV;
W_{Td} is the weight for Td;
W_{To} is the weight for To; and
W_{Te} is the weight for Te.

Each of the forty-first through forty-fourth aspects can be utilized in any of the other aspects discussed herein.

In a forty-fifth aspect, the apparatus for health monitoring can be configured so that the sensor data can be evaluated to determine whether the heart rate of the user is at or above a pre-selected anxiety threshold level and, in response to a determination that the heart rate is above the pre-selected anxiety threshold level, body movement sensor data can be evaluated to determine whether a frequency of motion is increasing beyond an anxiety threshold rate. In response to determining that the frequency of the motion increased beyond the anxiety threshold rate, an intervention to provide output to the user can be actuated. In a forty-sixth aspect, the output that is actuated can be a vibration generated by a vibration mechanism of the wearable device, music output by the wearable device or the input/output device, and/or audible sound output by a speaker of the input/output device or the wearable device. The server, wearable device, and/or the input/output device can be configured to perform such evaluation and determinations. For example, in a forty-seventh aspect, the wearable device can be configured to evaluate the sensor data and emit the output. In a forty-eighth aspect, the input/output device can evaluate the sensor data and emits the output. In a forty-ninth aspect, the server can evaluate the sensor data and the input/output device and/or the wearable device can emit the output.

In a fiftieth aspect, accelerometer data of the sensor data can be evaluated for a first tremor baseline time period to determine: (i) a number of tremors that happened within the first tremor baseline time period to determine a Tremor Count (T_{C}); (ii) a Tremor Duration (T_{D}) as an amount of time elapsed during occurrence and non-occurrence of a detected tremor for each tremor detected within the first tremor baseline time period based on the sensor data; (iii) a Tremor Amplitude (T_{A}) in the detected tremor within a single period of the tremor for each tremor detected within the first tremor baseline time period; and (iv) tremor frequency (T_{F}) as a number of tremor occurrences within the first tremor baseline time period.

In a fifty-first aspect, a first weight w1, a second weight w2, a third weight w3, and a fourth weight w4 can be determined to calculate a first baseline tremor score (first T_{S}) according to:

first T_{S}=(w1*T_{F}+w2*T_{A}+w3*T_{D}+w4*T_{C}).

In a fifty-second aspect, the input/output device, the server, or the wearable device can be configured to evaluate the accelerometer data to determine the first baseline tremor score.

In a fifty-third aspect, the apparatus can be configured so that, in response to input indicating a drug is taken by the user, the accelerometer data of the sensor data obtained after the drug is taken by the user is evaluated for a second tremor baseline time period to determine: (i) a number of tremors that happened within the second tremor baseline time period to determine a Tremor Count (T_{C}); (ii) a Tremor Duration (T_{D}) as an amount of time elapsed during occurrence and non-occurrence of a detected tremor for each tremor detected within the second tremor baseline time period based on the sensor data; (iii) a Tremor Amplitude (T_{A}) in the detected tremor within a single period of the tremor for each tremor detected within the second tremor baseline time period; and (iv) tremor frequency (T_{F}) as a number of tremor occurrences within the second tremor baseline time period. In a fifty-fourth aspect, a first weight w1, a second weight w2, a third weight w3, and a fourth weight w4 can be determined to calculate a second baseline tremor score (second T_{S}) according to:

second T_{S}=(w1*T_{F}+w2*T_{A}+w3*T_{D}+w4*T_{C}).

In a fifty-fifth aspect, the input/output device, the server, or the wearable device can be configured to evaluate the accelerometer data to determine the new second baseline tremor score. In a fifty-sixth aspect, the input/output device, the server, or the wearable device can be configured to compare the second baseline tremor score to the first baseline tremor score to evaluate efficacy of the drug. In a fifty-seventh aspect, the input/output device, the server, or the wearable device can be configured to generate output for suggesting a change to a dose of the drug and/or a frequency at which the drug is to be taken in response to determining that (i) the second baseline tremor score indicates a worse tremor condition as compared to the first baseline tremor score, (ii) the second baseline tremor score indicates a tremor condition that is the same as the first baseline tremor score; (iii) that the second baseline tremor score is higher than the first baseline tremor score, (iv) that the second baseline tremor score is within a pre-selected non-efficacy range of the first baseline tremor score, or (iv) that the second baseline tremor score differs from the first baseline tremor score by no more than a pre-selected efficacy value. In a fifty-eighth aspect, the output for suggesting a change to a dose of the drug and/or a frequency at which the drug is to be taken and/or the drug to be taken includes a graphical depiction and/or text. The output can also include audible output or other output.

In a fifty-ninth aspect, the server can be communicatively connectable to at least one data management device having health data of the user stored thereon to access the health data of the user to evaluate a sleep condition, a tremor condition, or a health condition of the user. An example of such a health data management device can include, for example, at least one electronic health record (EHR) device.

In a sixtieth aspect, the wearable device can be configured to record snoring and/or coughing that occurs while the user is asleep. In a sixty-first aspect, the server and/or the input/output device can be configured to identify a snoring and/or a coughing pattern from sensor data of the sensor array for predicting a sleep condition of the user based on the recorded snoring and/or coughing data of the sensor data. In a sixty-second aspect, the wearable device and/or the input/output device can be configured to output at least one audible and/or sensory output to the user while the user is still asleep to improve sleep quality and/or duration of sleep for the user while the user is asleep. Such output can be triggered via a communication from the server, input/output device, or wearable device.

In a sixty-third aspect, the wearable device and/or the input/output device can be configured to periodically evaluate sensor data to detect a heart rate, body movement and sweat of the user wearing the wearable device to determine whether the heart rate, body movement, and sweat exceed a pre-selected threshold sleep condition criteria so that at least one output is emitted by the wearable device and/or the input/output device to improve a duration and/or quality of sleep of the user in response to the pre-selected threshold sleep condition criteria being met or exceeded. In a sixty-fourth aspect, the output includes the wearable device vibrating via a vibration mechanism. In a sixty-fifth aspect, the output includes the wearable device or the input/output device triggering an audible output of at least one sound or music output via the input/output device. In a sixty-sixth aspect, the output can also include the wearable device vibrating via a vibration mechanism in combination with other output (e.g. audible and/or visual output).

In a sixty-seventh aspect, the apparatus for monitoring health can also include a docking station configured to communicatively connect the wearable device to the server and/or the input/output device and also recharge a battery of the wearable device. In a sixty-eight aspect, the docking station can be provided and be configured to communicatively connect the wearable device to the input/output device.

In a sixty-ninth aspect, the input/output device and/or the wearable device can be configured to output audible queries to solicit receipt of input from the user that include: (i) a series of nighttime questions output within a pre-selected time period of the user going to sleep; and/or (ii) a series of morning questions output within a pre-selected time period of the user waking up. The input/output device and/or wearable device can be configured to store user answers to the audible queries for evaluation of the sleep of the user. In a seventieth aspect, the input/output device and/or the wearable device can be configured to store an audio file defining a voice for the output of the audible queries so that the output of audible queries are output based on a stored audio file so that the audible queries are spoken by a voice of a relative or friend of the user. In some embodiments, such an audio file can be provided via the server.

In a seventy-first aspect, the input/output device, server, and/or wearable device can be configured to determine a quality of daytime activity (QODA) score. The QODA score can be determined based on the stored sensor data in some embodiments.

In a seventy-second aspect, the QODA score can be determined based on a formula of:

QODA=Sleep Score+mind,body and diet (MBD)Score;

QODA=Sleep Score+MBD Score+Activity Score;

QODA=Sleep Score*W_{QODASS}+MBD Score*W_{QODAMBD}; or

QODA=Sleep Score*W_{QODASS}+MBD Score*W_{QODAMBD}+Activity Score*W_{QODAAct}

where:
W_{QODASS} is a QODA weight for the Sleep Score;
W_{QODAMBD} is a weight for the MBD Score; and
W_{QODAAct} is a weight for the Activity Score.

In a seventy-third aspect, the MBD score can be determined from:

MBD score=MB*w1_{MBD}+D*w2_{MBD}

where:
MB is a mind and body score based on subjective input the user provided;
D is a diet score based on dietary information of the user;
w1_{MBD} is a weight for the MB score; and
w2_{MBD} is a weight to weigh the diet score D.

In a seventy-fourth aspect, the Sleep Score can be determined from:

Sleep Score=W_{LS}*LS+W_{DS}*DS+W_{RS}*RS+W_{BMsleep}*BMₛₗₑₑₚ,+W_{ENV}*ENV+W_{Td}*Td+W_{To}*To+W_{Te}*Te;

wherein:
LS is a value for an amount of time the user was determined to be in a light sleep state;
DS is a value for an amount of time the user was determined to be in a deep sleep state;
RS is value for an amount of time the user was determined to be in a REM sleep state;
Td is a value for duration of total sleep time of the user;
To is a value for a determined sleep onset for the sleep;
Te is a value for the determined sleep efficiency for the sleep;
BMₛₗₑₑₚ is a value for the detected body movement of the user during the sleep;
ENV is a value for the detected environment during the sleep;
W_{LS} is the weight for LS;
W_{DS} is the weight for DS;
W_{RS} is the weight for RS;
W_{BMsleep} is the weight for BMₛₗₑₑₚ,
W_{ENV} is the weight for ENV;
W_{Td} is the weight for Td;
W_{To} is the weight for To; and
W_{Te} is the weight for Te.

It should be appreciated that any of the forty-fifth through seventy-fourth aspects can be combined with the first aspect in any combination and can also be combined with any of the other aspects discussed herein.

In a seventy-fifth aspect, a method of monitoring a health condition of a user can include obtaining sensor data via a sensor array when a user wears a wearable device having the sensor array, analyzing the sensor data to track a condition of the user, and generating output to suggest a change to help the user improve the condition or maintain the condition based on the analyzed sensor data. An embodiment of the apparatus for monitoring health can be configured to utilize the method.

In a seventy-sixth aspect, the method can be configured so that the analyzing of the sensor data to track the condition of the user is performed to determine a first baseline for the condition. The server, input/output device, or the wearable device of the apparatus for monitoring health can perform such analyzing of the sensor data in some embodiments.

In a seventy-seventh aspect, the method can include responding to a first input indicating that a drug at a first dosage is being taken by the user by comparing the baseline for the condition with the sensor data obtained after the receipt of the first input to determine whether the condition has improved. The wearable device, input/output device, or server can be configured to implement such a response to the first input.

In a seventy-eight aspect, the method can include responding to a determination that the condition has not improved within a pre-specified improvement time period after receipt of the first input by suggesting a change to the user for adjusting a frequency of taking the drug and/or adjusting a dosage of the drug to a second dosage that differs from the first dosage and/or adjusting the drug to a different drug indicated for the condition. As discussed herein, the second dosage may be a higher dosage than the first dosage or a lower dosage than the first dosage. The frequency adjustment for the drug can be taking the drug more frequently or less frequently. For instance, frequency adjustment may occur by the same dosage of the drug or a different dosage of the drug being taken ever 2-4 hours instead of once a day or being taken 4-6 hours instead of being taken every 2-4 hours, or being taken every 6-12 hours instead of once a day, etc. Other types of frequency adjustments can also be provided. In some embodiments, the wearable device, input/output device, or server can be configured to implement such a response to the determination that the condition has not improved.

In a seventy-ninth aspect, the method can also include responding to a second input indicating that a change to the drug being taken by the user has occurred by comparing the baseline for the condition with the sensor data obtained after the receipt of the second input to determine whether the condition has improved. In some embodiments, the wearable device, input/output device, or server can be configured to implement such a response to the second input.

In an eightieth aspect, the method can also include responding to a determination that the condition has not improved within a pre-specified improvement time period after receipt of the second input by suggesting a change to the user for adjusting a frequency of taking the drug and/or adjusting a dosage of the drug to a third dosage that differs from the first dosage and/or adjusting the drug to a third different drug indicated for the condition. The third dosage may be a higher dosage than the first dosage or a lower dosage than the first dosage. The third dosage may be a higher dosage than the second dosage or a lower dosage than the second dosage as well. The frequency adjustment for the drug can be taking the drug more frequently or less frequently. For instance, frequency adjustment may occur by the same dosage of the drug or a different dosage of the drug being taken ever 2-4 hours instead of once a day or being taken 4-6 hours instead of being taken every 2-4 hours, or being taken every 6-12 hours instead of once a day, etc. Other types of frequency adjustments can also be provided. In some embodiments, the wearable device, input/output device, or server can be configured to implement such a response to the determination that the condition has not improved.

In an eighty-first aspect, the condition can be a tremor condition, epilepsy, a sleep condition, an Alzheimer's disease condition, a neurological disorder, a neurodegenerative disease associated with a tremor or for which the tremor is a symptom, multiple sclerosis, stroke, traumatic brain injury, Parkinson's disease, ADHD, dementia, Alzheimer's disease, the condition is a result of use of a medicine, the condition is a result of alcohol abuse, the condition is a withdrawal of a drug, the condition is a thyroid condition, the condition is an overactive thyroid, the condition is a liver condition, the condition is fatty liver disease, the condition is kidney failure, the condition is anxiety or the condition is panic.

In an eighty-second aspect, the analyzing of the sensor data to track the condition of the user can include determining a baseline level of sleep for the user. In an eighty-second aspect, the method can also include monitoring sleep of the user based on the sensor data obtained when the user wears the wearable device having the sensor array while sleeping to determine one or more time durations at which the user was in a light sleep state during the sleep, a deep sleep state during the sleep, and/or a rapid eye movement (REM) sleep state during the sleep. In an eighty-fourth aspect, the monitoring of the sleep can be performed such that it is automatically determined when the user has woken up to stop monitoring of the sleep of the user. In an eighty-fifth aspect, the monitoring of the sleep can be performed to automatically determine when the user has begun attempting to go to sleep to start monitoring of the sleep of the user. Embodiments of the apparatus for monitoring health can be configured to implement embodiments of the eighty-second through eighty-fifth aspects.

In an eighty-sixth aspect, the method can include calculating: (i) a total time slept during a monitoring of the sleep, (ii) a time of sleep onset indicating a time it took the user to fall asleep after being detected as attempting to go to sleep, and/or (iii) a sleep efficiency indicating an amount of time the user was asleep during the total time the sleep of the user was monitored. In some embodiments, only one of these parameters (i)-(iii) can be calculated, only two of these parameters (i)-(iii) can be calculated or at least all of parameters (i)-(iii) can be calculated. Embodiments of the apparatus can be configured to implement such an aspect.

In an eighty-seventh aspect, the method can include determining (i) an amount of time during the monitored sleep that the user was in the light sleep state based on the sensor data, (ii) an amount of time during the monitored sleep that the user was in the deep sleep state based on the sensor data, and/or (iii) an amount of time during the monitored sleep that the user was in the REM sleep state based on the sensor data. In some embodiments, only one of these parameters (i)-(iii) can be determined. In other embodiments, only two of these parameters (i)-(iii) can be determined. In yet other embodiments, at least all of parameters (i)-(iii) can be determined.

In an eighty-eighth aspect, the method can include determining a sleep score for the user based on the sensor data obtained from monitoring of the sleep of the user. In an eighty-ninth aspect, the sleep score can be determined in accordance with a sleep score formula:

Sleep Score=W_{LS}*LS+W_{DS}*DS+W_{RS}*RS+W_{BMsleep}*BMₛₗₑₑₚ,+W_{ENV}*ENV+W_{Td}*Td+W_{To}*To+W_{Te}*Te;

wherein:
LS is a value for an amount of time the user was determined to be in a light sleep state;
DS is a value for an amount of time the user was determined to be in a deep sleep state;
RS is value for an amount of time the user was determined to be in a REM sleep state;
Td is a value for duration of total sleep time of the user;
To is a value for a determined sleep onset for the sleep;
Te is a value for the determined sleep efficiency for the sleep;
BMₛₗₑₑₚ is a value for the detected body movement of the user during the sleep;
ENV is a value for the detected environment during the sleep;
W_{LS} is the weight for LS;
W_{DS} is the weight for DS;
W_{RS} is the weight for RS;
W_{BMsleep} is the weight for BMₛₗₑₑₚ,
W_{ENV} is the weight for ENV;
W_{Td} is the weight for Td;
W_{To} is the weight for To; and
W_{Te} is the weight for Te.

In a ninetieth aspect, the method can also include determining whether the heart rate of the user is at or above a pre-selected anxiety threshold level based on the sensor data and, in response to a determination that the heart rate is above the pre-selected anxiety threshold level, evaluating body movement sensor data to determine whether a frequency of motion is increasing beyond an anxiety threshold rate. In response to determining that the frequency of the motion increased beyond the anxiety threshold rate, an intervention to provide output to the user can be actuated. Embodiments of the apparatus can be configured to implement such an aspect.

In a ninety-first aspect, the method can be implemented so that the output provided in response to determining that the frequency of the motion increased beyond the anxiety threshold rate is a vibration generated by a vibration mechanism of the wearable device, music output by the wearable device or an input/output device, and/or audible sound output by a speaker of the input/output device or the wearable device.

In a ninety-second aspect, the method can include evaluating accelerometer data of the sensor data for a first tremor baseline time period to determine: (i) a number of tremors that happened within the first tremor baseline time period to determine a Tremor Count (T_{C}); (ii) a Tremor Duration (T_{D}) as an amount of time elapsed during occurrence and non-occurrence of a detected tremor for each tremor detected within the first tremor baseline time period based on the sensor data; (iii) a Tremor Amplitude (T_{A}) in the detected tremor within a single period of the tremor for each tremor detected within the first tremor baseline time period; and (iv) tremor frequency (T_{F}) as a number of tremor occurrences within the first tremor baseline time period.

In a ninety-third aspect, the method can be implemented for determining a first weight w1, a second weight w2, a third weight w3, and a fourth weight w4 to calculate a first baseline tremor score (first T_{S}) according to:

first T_{S}=(w1*T_{F}+w2*T_{A}+w3*T_{D}+w4*T_{C}).

In a ninety-fourth aspect, the method can also include the accelerometer data of the sensor data obtained after the drug is taken by the user being evaluated for a second tremor baseline time period in response to input indicating a drug is taken by the user. The evaluation of the accelerometer data of the sensor data can be performed to determine: (i) a number of tremors that happened within the second tremor baseline time period to determine a Tremor Count (T_{C}); (ii) a Tremor Duration (T_{D}) as an amount of time elapsed during occurrence and non-occurrence of a detected tremor for each tremor detected within the second tremor baseline time period based on the sensor data; (iii) a Tremor Amplitude (T_{A}) in the detected tremor within a single period of the tremor for each tremor detected within the second tremor baseline time period; and (iv) tremor frequency (T_{F}) as a number of tremor occurrences within the second tremor baseline time period.

In a ninety-fifth aspect, the method can also include determining a first weight w1, a second weight w2, a third weight w3, and a fourth weight w4 a to calculate a second baseline tremor score (second T_{S}) according to:

second T_{S}=(w1*T_{F}±w2*T_{A}±w3*T_{D}±w4*T_{C}).

In a ninety-sixth aspect, the method can also include comparing the second baseline tremor score to the first baseline tremor score to evaluate efficacy of the drug. In a ninety-seventh aspect, the method can include changing a dose of the drug and/or a frequency at which the drug is to be taken in response to determining that (i) the second baseline tremor score indicates a worse tremor condition as compared to the first baseline tremor score, (ii) the second baseline tremor score indicates a tremor condition that is the same as the first baseline tremor score; (iii) that the second baseline tremor score is higher than the first baseline tremor score, (iv) that the second baseline tremor score is within a pre-selected non-efficacy range of the first baseline tremor score, or (iv) that the second baseline tremor score differs from the first baseline tremor score by no more than a pre-selected efficacy value.

In a ninety-eight aspect, the method can include recording snoring and/or coughing that occurs while the user is asleep. In a ninety-ninth aspect, the method can include identifying a snoring and/or a coughing pattern from recording of the snoring and/or coughing and the sensor data to predict a sleep condition of the user based on the recorded snoring and/or coughing Ie a one hundredth aspect, the method can also include emitting at least one audible and/or sensory output to the user while the user is still asleep to improve sleep quality and/or duration of sleep for the user while the user is asleep.

In a one hundred and first aspect, the method can also include periodically evaluating the sensor data to detect a heart rate, body movement and sweat of a user wearing the wearable device to determine whether the heart rate, body movement, and sweat exceed a pre-selected threshold sleep condition criteria so that at least one output is emitted by a wearable device and/or an input/output device to improve a duration and/or quality of sleep of the user in response to the pre-selected threshold sleep condition criteria being met or exceeded. In a one hundred and second aspect, the output can include the wearable device vibrating via a vibration mechanism. In a one hundred and third aspect, the output can also (or alternatively) include the wearable device or the input/output device triggering an audible output of at least one sound or music output via the wearable device and/or the input/output device.

In a one hundred and fourth aspect, the method can include querying the user (i) a series of nighttime questions output within a pre-selected time period of the user going to sleep and/or (ii) a series of morning questions output within a pre-selected time period of the user waking up. The method can also include storing user answers to the audible queries for evaluation of the sleep of the user. In a one hundred and fifth aspect, the method can also include storing an audio file defining a voice for the output of the audible queries so that the output of audible queries are output based on a stored audio file so that the audible queries are spoken by a voice of a relative or friend of the user.

In a one hundred and sixth aspect, the method can include determining a quality of daytime activity (QODA) score based on the sensor data. In a one hundred and seventh aspect, the QODA score can be determined based on a formula of:

QODA=Sleep Score+mind,body and diet (MBD) Score;

QODA=Sleep Score+MBD Score+Activity Score;

QODA=Sleep Score*W_{QODASS}+MBD Score*W_{QODAMBD}; or (

QODA=Sleep Score*W_{QODASS}+MBD Score*W_{QODAMBD}+Activity Score*W_{QODAAct}

where:
W_{QODASS} is a QODA weight for the Sleep Score;
W_{QODAMBD} is a weight for the MBD Score; and
W_{QODAAct} is a weight for the Activity Score.

In a one hundred and eighth aspect, the MBD score can be determined from:

MBD score=MB*w1_{MBD}+D*w2_{MBD}

where:
MB is a mind and body score based on subjective input the user provided;
D is a diet score based on dietary information of the user;
w1_{MBD} is a weight for the MB score;
w2_{MBD} is a weight to weigh the diet score D.

In a one hundred and ninth aspect, the Sleep Score can be determined from:

Sleep Score=W_{LS}*LS+W_{DS}*DS+W_{RS}*RS+W_{BMsleep}*BMₛₗₑₑₚ,W_{ENV}*ENV+W_{Td}*Td+W_{To}*To+W_{Te}*Te;

wherein:
LS is a value for an amount of time the user was determined to be in a light sleep state;
DS is a value for an amount of time the user was determined to be in a deep sleep state;
RS is value for an amount of time the user was determined to be in a REM sleep state;
Td is a value for duration of total sleep time of the user;
To is a value for a determined sleep onset for the sleep;
Te is a value for the determined sleep efficiency for the sleep;
BMₛₗₑₑₚ is a value for the detected body movement of the user during the sleep;
ENV is a value for the detected environment during the sleep;
W_{LS} is the weight for LS;
W_{DS} is the weight for DS;
W_{RS} is the weight for RS;
W_{BMsleep} is the weight for BM
W_{ENV} is the weight for ENV;
W_{Td} is the weight for Td;
W_{To} is the weight for To; and
W_{Te} is the weight for Te.

Embodiments of the apparatus for monitoring health can be configured to implement any of the seventy-fifth through one hundred and ninth aspects. Moreover, it should be appreciated that any of the seventy-sixth through one hundred and ninth aspects can be combined with each other in different embodiments of the method.

A wearable device is also provided. Embodiments of the wearable device can be included in the apparatus for monitoring health. The wearable device can include a processor, a non-transitory computer readable medium connected to the processor, and a sensor array connected to the processor and/or the non-transitory computer readable medium.

In some embodiments of the wearable device, the sensor array of the wearable device can include an optical sensor to monitor heart rate and blood oxygen content; a temperature sensor to measure temperature of a user wearing the wearable device; a microphone to detect audible noise during sleep; and a sweat sensor to measure sweat of the user. Other embodiments can utilize different sensor arrays having additional sensors, less sensors, or different sensors.

Embodiments of the wearable device can include a housing. The sensor array can be attached to the housing. The housing can also have an identifier attached thereto or defined thereon.

The wearable device can include a power unit. An example of a power unit can include a rechargeable battery attached to or within the housing.

Embodiments of the wearable device can also include at least one data transmission interface or at least one wireless transceiver unit connected to the processor.

Embodiments of the wearable device can be configured to record snoring and/or coughing that occurs while the user is asleep for storage and transmission. The wearable device can be configured to send such data to another device (e.g. a server or an input/output device). Embodiments of the wearable device can also (or alternatively) be configured to identify a snoring and/or a coughing pattern from data of the sensor array for predicting a sleep condition of the user.

Embodiments of the wearable device can be configured to periodically evaluate sensor data to detect a heart rate, body movement and sweat of a user wearing the wearable device and, upon a determination that the heart rate, body movement, and sweat exceed a pre-selected threshold sleep condition criteria, cause at least one output to be emitted to improve a duration and/or quality of sleep of the user. This output can include the wearable device vibrating via a vibration mechanism in some embodiments. The output can also (or alternatively) include e wearable device triggering an audible output of at least one sound or music. The sound or music can be output via the wearable deice or an input/output device communicatively connectable to the wearable device in some embodiments.

Embodiments of an apparatus for monitoring at least one user of a wearable device is also provided. Some embodiments can utilize aspects of the apparatus for monitoring health discussed herein. In some embodiments, the wearable device can be communicatively connected to an input/output device and/or a central server for transmission of data collected via the sensor array to the central server, for example.

Embodiments of the apparatus for monitoring at least one user of a wearable device can also include at least one data management device having health data of the user stored thereon. The central server can be communicatively connected to the least one data management device to access the health data of the user to evaluate a sleep condition or health condition of the user. Examples of at least one data management device having health data of the user stored thereon can include, for example, an electronic health record (EHR) device.

Embodiments of the apparatus for monitoring at least one user of a wearable device can be configured such that the wearable device is configured to record snoring and/or coughing that occurs while the user is asleep for transmission to the central server. In some embodiments, the central server can be configured to identify a snoring and/or a coughing pattern from data of the sensor array for predicting a sleep condition of the user.

Embodiments of the apparatus for monitoring at least one user of a wearable device can also include a docking station configured to communicatively connect the wearable device to the central server and/or recharge a battery of the wearable device.

A method of evaluating sleep of a user is also provided. Embodiments of the method can include providing an embodiment of the wearable device or an embodiment of the apparatus for monitoring health.

Embodiments of the method of evaluating sleep can also include recording snoring and/or coughing that occurs while the user is asleep via the wearable device for transmission of data of the recorded snoring and/or coughing to a central server. The central server can identify a snoring and/or a coughing pattern from the data of the recorded snoring and/or coughing to detect or predict a sleep condition of the user.

Embodiments of the method of evaluating sleep can also include recording heart rate, sweat and body movement data, determining from the recorded heart rate, sweat and body movement data whether the user has exceeded a pre-selected sleep criteria threshold; and outputting at least one audible and/or sensory output to the user while the user is still asleep to improve sleep quality and/or duration of sleep for the user while the user is asleep.

It should be appreciated that embodiments of the method of evaluating sleep can also be configured to utilize different aspects of methods or apparatuses discussed herein.

Other details, objects, and advantages of the electronic devices and communication systems that can be configured to facilitate the monitoring of a patient's sleep and/or the efficacy of a sleep treatment for a patient who has been diagnosed as having a sleep related health issue as well as methods of making and using the same and methods of evaluating a sleep treatment and methods of facilitating the diagnosis of a sleep related health issue will become apparent as the following description of certain exemplary embodiments thereof proceeds. Similarly, other details, objects, and advantages of the electronic devices and communication systems that can be configured to facilitate the monitoring of a patient's tremors and/or the efficacy of a treatment for a patient experiencing tremors as well as methods of making and using the same and methods of evaluating a treatment for a neurological disorder or neurodegenerative diseases or other condition associated with tremors and methods of facilitating the diagnosis of a tremor related health issue will become apparent as the following description of certain exemplary embodiments thereof proceeds.

Other details, objects, and advantages of the electronic devices and communication systems that can be configured to facilitate the monitoring of a patient's health and/or the efficacy of a treatment (e.g. use of drug, behavioral change of the patient, etc.) for a patient to improve the patient's health as well as methods of making and using the same will become apparent as the following description of certain exemplary embodiments thereof proceeds.

### BRIEF DESCRIPTION OF THE DRAWINGS

Exemplary embodiments of electronic devices and communication systems as well as methods of making and using the same and methods of evaluating a sleep treatment and methods of facilitating the diagnosis of a sleep related health issue are shown in the drawings included herewith. It should be understood that like reference characters used in the drawings may identify like components.

FIG. 1 is a block diagram of a first exemplary apparatus configured to facilitate the monitoring of a patient's health. This can include, for example monitoring a patient's health data related to the patient's sleep and/or the efficacy of a sleep treatment for a patient who has been diagnosed as having a sleep related health issue. This can also (or alternatively) include monitoring of the patient's health related to a condition of the patient that can include a tremor condition symptom. This can also (or alternatively) include monitoring of the patient's health for use of the monitored data to evaluate how a treatment may be affecting the patient's health (e.g. use of drug, behavioral change of the patient, etc.). Different platform configurations for a first platform 8 and a second platform 9 are illustrated in broken line in FIG. 1 as different platform configurations that can be utilized in some embodiments of the apparatus.

FIG. 2 is a perspective view of a first exemplary embodiment of a wearable electronic device that can be included in the exemplary apparatus shown in FIG. 1. The electronic device is illustrated in a first configuration in which the electronic device can be worn by a user (e.g. around the user's wrist, upper arm, lower arm, lower leg, upper leg, chest, waist, ankle, arm, leg, or neck) in FIG. 2.

FIG. 3 is a schematic view of an exemplary battery assembly that can be utilized in conjunction with embodiments of the electronic device 3 shown in FIGS. 1 and 2.

FIG. 4 is an exploded view of the first exemplary embodiment of the wearable electronic device that can be included in the exemplary apparatus shown in FIG. 1. The electronic device is illustrated in a second configuration in FIG. 4 in which the electronic device can be released from a user (e.g. be unclasped from the user's wrist, ankle, arm, leg, chest, waist, upper arm, lower arm, upper leg, lower leg, or neck).

FIG. 5 is a schematic block diagram of an exemplary circuit board arrangement for the first exemplary embodiment of the wearable electronic device.

FIG. 6 is a flow chart illustrating an exemplary embodiment of a method for collecting patient data, analysis and use of the data that can be utilized by the first exemplary embodiment of the electronic device and/or components of the apparatus shown in FIG. 1.

FIG. 7 is a flow chart illustrating an exemplary embodiment of a method for utilizing sensor data from the first exemplary embodiment of the electronic device or at least one exemplary component of the apparatus shown in FIG. 1.

FIG. 8 is a flow chart illustrating a method by which the exemplary apparatus of FIG. 1 can be configured for operation.

FIG. 9 is a perspective view of the first exemplary embodiment of the wearable electronic device configured to be worn near an ankle of a patient.

FIG. 10 is a perspective view of a first exemplary embodiment of the wearable electronic device in a configuration so that the device can be positioned on or near the chest of a patient.

FIG. 11 is perspective view of the first exemplary embodiment of the wearable electronic device in the first configuration.

FIG. 12 is a flow chart illustrating a first exemplary embodiment of a method for integrating subjective and objective patient data to detect a patient pattern and suggest a behavior alteration to help the patient detect the pattern and take action to avoid a possible negative condition that may result from the detected pattern.

FIG. 13 is a flow chart illustrating a second exemplary embodiment of a method for integrating subjective and objective patient data to detect a patient pattern and suggest a behavior alteration to help the patient detect the pattern and take action to avoid a possible negative condition that may result from the detected pattern.

FIG. 14 is a flow chart illustrating an exemplary embodiment of a method for detecting a possible sleep condition that can trigger sleep monitoring as well as triggering utilization of a method that integrates subjective and objective patient data to detect a pattern of conduct by the patient that could contribute to the detected sleep condition.

FIG. 15 is a flow chart illustrating an exemplary embodiment of a method that can facilitate utilization of personalized voice data for audibly communicating prompts, reminders, questions, or other audible output to a user.

FIG. 16 is a flow chart illustrating an exemplary method of monitoring sleep and responding to the monitored sleep to account for a detected patient condition to provide at least one type of output (e.g. music, white noise, vibration, etc.) that can facilitate improvement in the patient's sleep (e.g. improve the quality of sleep and/or duration of time the patient is asleep).

FIG. 17 is a flow chart illustrating an exemplary embodiment of process for prompting a user to provide subjective and objective data via visual, video and/or audio output at different times to solicit input via at least one input device for collecting subjective user data related to the monitoring of the user's sleep and storing and/or transmitting the user provided input to help monitor at least one health condition of the user.

FIG. 18 is a block diagram of the first exemplary embodiment of the wearable electronic device that can be included in the exemplary apparatus shown in FIG. 1.

FIG. 19 is side view of the first exemplary embodiment of the wearable electronic device in the first configuration.

FIG. 20 is a perspective of the of the exemplary embodiment of the wearable electronic device positioned for battery charging and communicative connection with a first exemplary embodiment of the docking station 5 the that can be included in the exemplary apparatus shown in FIG. 1.

FIG. 21 is a flow chart illustrating an exemplary process for monitoring data related to at least one health condition of a user and transmitting data related to the monitored data.

FIG. 22 is a flow chart illustrating an exemplary process for detection of a tremor of a user.

FIG. 23 is a flow chart illustrating an exemplary process for monitoring a tremor of a user and evaluating it.

FIG. 24 is a chart illustrating a scale for allocation of a user's subjective data input.

FIG. 25 is a flow chart illustrating an exemplary process for calculating a mind, body, and diet (MBD) score. The MBD score can also be considered a type of Emotion and Diet score.

FIG. 26 is a chart illustrating a scale for allocation of a user's subjective data input concerning the user's diet.

FIG. 27 is a flow chart illustrating an exemplary process for calculating a sleep score using data obtained from monitoring of one or more health conditions of the user.

FIG. 28 is a schematic diagram illustrating an exemplary display of a graphical user interface (GUI) that can be generated for display by an input/output device 13 (e.g. smart phone, tablet, laptop computer, personal computer, smart speaker, etc.), a wearable device 3, and/or docking station 5.

FIG. 29 is a flow chart illustrating an exemplary process for determining sleep parameters for a patient based on sensor data obtained via the wearable device 3.

FIG. 30 is a flow chart illustrating exemplary process for determining sleep parameters of a user via sensor data obtained from the wearable device 3 for evaluation of whether the user is in a sleep state or is awake.

FIG. 31 is a flow chart illustrating an exemplary process for determining whether the user is awake based on the sensor data.

FIG. 32 is a flow chart illustrating an exemplary process for determining whether the use is in a light sleep state based on the sensor data.

FIG. 33 is a flow chart illustrating an exemplary process for determining whether the user is in a deep sleep state based on the sensor data.

FIG. 34 is a flow chart illustrating an exemplary process for determining whether the user is in a rapid eye movement (REM) sleep state based on the sensor data.

### DETAILED DESCRIPTION OF EXEMPLARY EMBODIMENTS

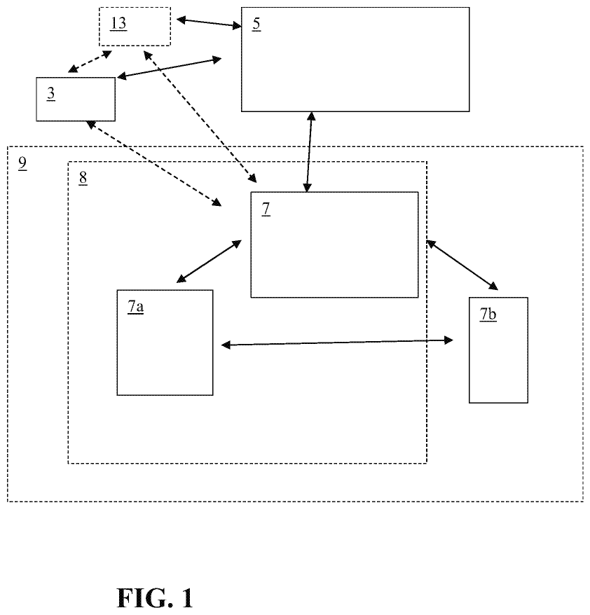

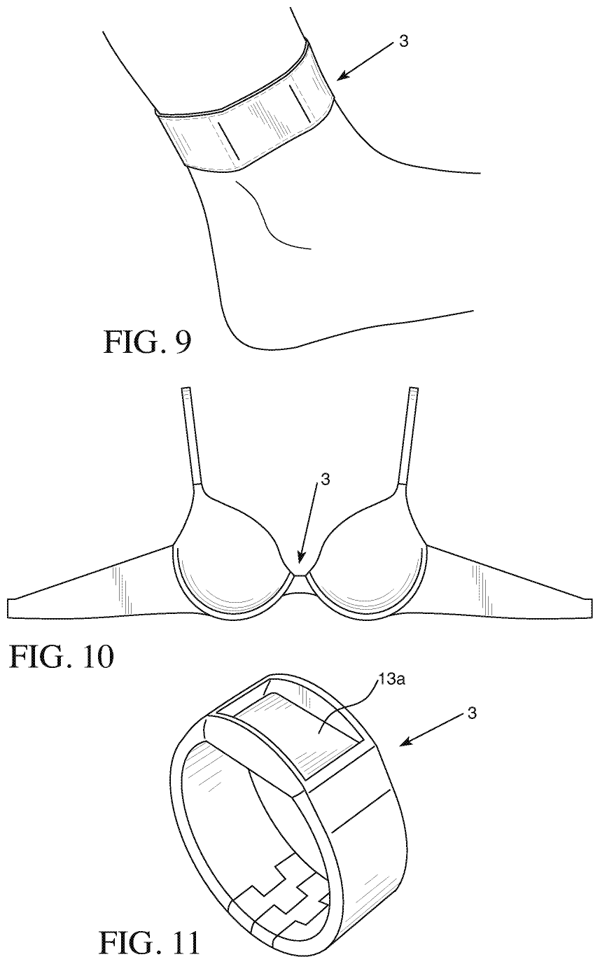

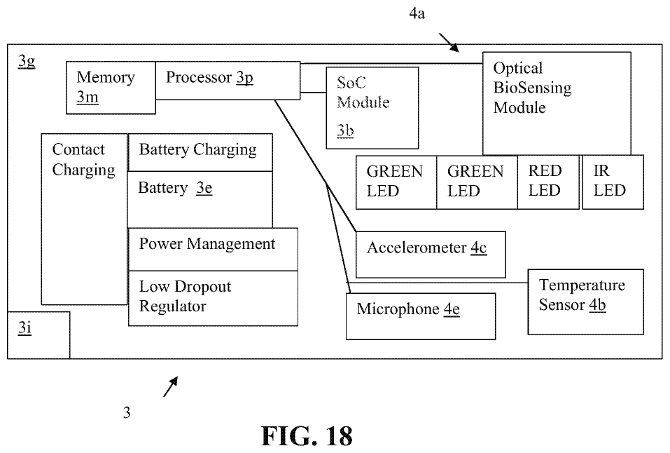

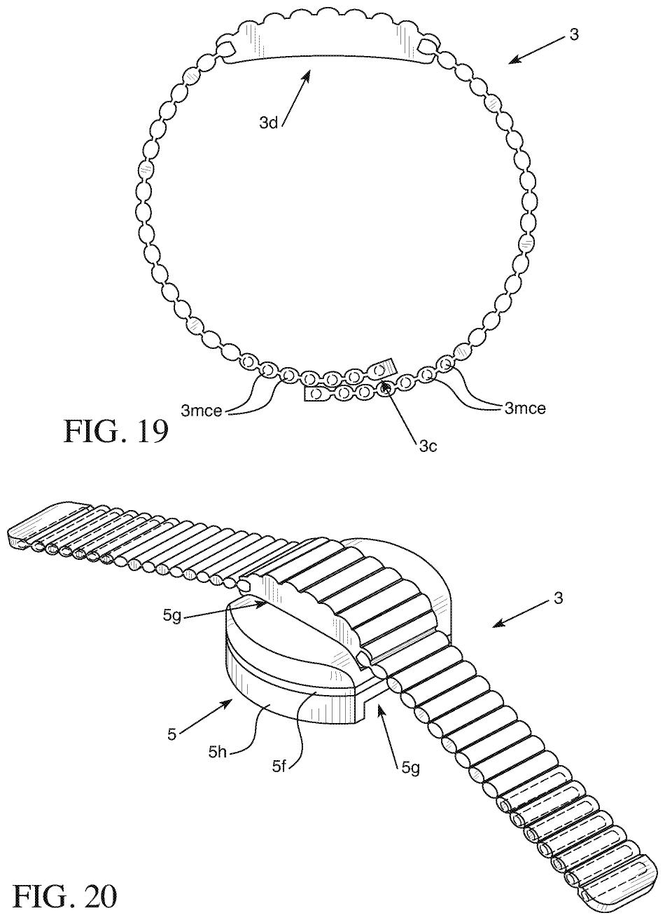

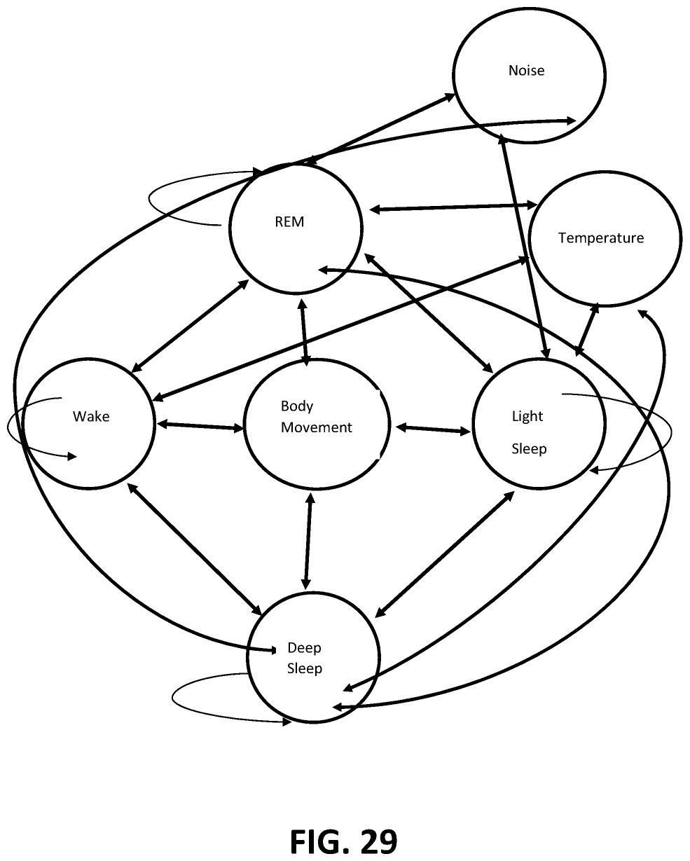

Referring to FIGS. 1-34, an apparatus 1 can include a wearable device 3 that can be structured as an electronic device that is adjustable between a first position in which the device can be attached to a user's body (e.g. clasped to form a band, ring or other annular structure that can encircle a user's arm, leg, upper leg, lower leg, upper arm, lower arm, wrist, ankle, chest, waist, neck, etc. as can be appreciated from FIGS. 9, 11 and 19, for example) and a second position in which the device can be separated from a user (e.g. taken off a user's body, taken off a user's arm, upper arm, lower arm, upper leg, lower leg, waist, chest, wrist, leg, ankle, neck, etc.). As can be seen in FIG. 10, embodiments of the wearable device 3 can also be integrated into a garment for positioning of the wearable device on the patient (e.g. near a chest of the patient or on another part of the patient). The garment can be a conductive garment or a non-conductive garment. Straps of the wearable device can also include attachable extension straps to permit the wearable band to be worn on larger parts of a user's body or to better accommodate people of different sizes.

The wearable device 3 can include at least one processor 3p (e.g. a microcontroller, a central processing unit processor assembly, an array of processors, a core processor, interconnected processors, etc.) that is connected to non-transitory memory 3m (e.g. flash memory) or other type of non-transitory computer readable medium, and at least one transceiver unit 3b (e.g. a wireless network transceiver, a cellular network transceiver, a Bluetooth transceiver, a universal serial bus (“USB”) transceiver, etc.). The wearable device 3 can also include an array 3a of sensors 4 that are communicatively connected to the non-transitory computer readable medium (e.g. memory 3m) and/or one or more processors 3p.

Each of the sensors 4 of the wearable device 3 can be configured to detect or measure at least one physical property of a patient. For measuring a patient's sleep, the array and positioning of the sensors can be paramount to gather critical information at relatively low costs. There can be challenges with incorporating the array of sensors into the wearable device. For instance, while many sensors can be produced in very small sizes, housing additional components needed to measure, amplify, convert, process, and transmit at least one signal generated by the sensor can pose challenges to achieving small form factors or placing sensors in ideal locations. Moreover, components of such sensors may have limited life and need frequent replacing. We have developed solutions to such issues as may be appreciated from the exemplary embodiments discussed herein.

The wearable device 3 can also include a power source 3e that can provide a source of power to the electrical components and sensors. The power source can be a rechargeable battery or other type of battery, for example, that can provide a source of voltage to power the device and the components of the device. The power source 3e can also include a power management module that can include a low dropout regulator (LDO) as well as contact charging and battery charging modules configured to facilitate different charging mechanisms for charging the battery (e.g. charging via UBS connection, wireless charging, magnetic resonant coupling for charging of the battery, etc.).

The wearable device 3 can be communicatively connectable to a docking station 5 and/or an access point (e.g. a wireless access point). In some embodiments, the docking station 5 can be configured as a recharging station for the wearable device 3 that can recharge the battery of the wearable device and also function as an access point that facilitate a communication connection of the wearable device 3 with a network so that the wearable device 3 is able to be communicatively connected to a remote first platform 8 and/or a remote second platform 9 that can include at least one central server 7 and/or other communication devices. The docking station 5 can also be configured to receive sensor data or other data from the wearable device 3 while the wearable device is connected to the docking station for recharging or within a proximity of a near field wireless communication connection for subsequently using that data and/or forwarding that data to the central server 7.

In some embodiments, the docking station 5 and wearable device 3 can be configured so that whenever the wearable device 3 is charging and/or within a proximity of the docking station 5, the wearable device 3 transmits the sensor data to the docking station 5 for subsequent processing and/or forwarding to central server 7. The transmitted sensor data can be all the data stored in the memory of the wearable device 3 or can be all the sensor data that is stored in the memory of the wearable device that had not previously been transmitted to the docking station. In yet other embodiments, the transmitted data can be a particular sub-type or class of data of the wearable device that is automatically sent by the wearable device to the docking station 5 or is sent by the wearable device in response to a request for such data received from the docking station.

In some embodiments, the network can represent a combination of connections and protocols that support communication between two connected devices that may be a Bluetooth network, a Wi-Fi network, or a combination of such networks and may operate in wireless frequencies or frequency ranges including 2.4 GHz and 5 GHz internet, near-field communication, Z-Wave, ZigBee etc. The network can include or help ensure creation and maintenance of a communication channel or a gateway channel capable of transferring data between connected devices.

In some embodiments, the wearable device 3 can be configured to transmit sensor data to a central server 7 so that the sensor data is storable in at least one wearable device data management device 7a and also storable in a patient data management device 7b that may be overseen by a care provider or other entity that has control over a patient's health related data. The forwarding of such data can be provided via a communication connection the wearable device 3 directly has with the docking station 5 such that data is sent to the central server 7 via the docking station 5. The forwarding of such data can also (or alternatively) be provided via a communication connection the wearable device 3 directly has with an input/output device 13 (e.g. a smart phone, smart speaker, laptop computer, etc.) such that data is sent to the central server 7 via the input/output device 13.

It should be appreciated that the central server 7, wearable device data management device 7a and patient data management device 7b can each be configured as computer device that has at least one processor connected to at least one non-transitory computer readable medium (e.g. memory, a hard drive, etc.) and at least one transceiver unit (e.g. a network transceiver, etc.). Each data management device can be configured as a database server, for example. The central server 7 can be configured as a host server that hosts services provided to the user of the wearable device 3 via communications exchanged between the central server 7 and the wearable device 3, docking station 5, and/or at least one input/output device 13 (e.g. smart phone, smart speaker, tablet, laptop computer, personal computer, etc.).

The patient data management device 7b can be a server computer device or a collection of server computer devices that are configured to retain a patient's health records by a care provider (e.g. electronic health records (EHRs)). The patient data management device 7b can be remote from the central server 7 or can be a component of the platform 9 having the central server 7 and the wearable device data management device 7b (e.g. within the same corporate network environment, within the same local area network, etc.). The wearable device data management device 7a can be remote from the central server 7 or can be a component of the first platform 8 and/or the second platform 9.

In some embodiments, the central server 7 can be an array of servers that includes at least one data management device 7a for the collection of sensor data. The central server 7 can also be configured to forward the collected sensor data to a remote patient data management device 7b that may be remote from the first platform 8 of the central server 7 (e.g. for embodiments in which the patient data management device 7b is part of a care provider computer system) via at least one network connection (e.g. via an encrypted internet transmission, etc.).

As discussed herein, embodiments of the first platform 8 and second platform 9 can also include other elements of a communication system. For instance, embodiments of these platforms can include at least one gateway or session border control device to facilitate a network connection between the wearable device 3 and the central server 7 and/or the central server 7 and the wearable device data management server 7a and/or the central server and the patient data management device 7b and/or the input/output device 13 and the central server 7 and/or the docking station 5 and the central server 7. In some embodiments, the wearable device 3 can be configured to transmit encrypted data to and receive encrypted data from the central server 7 via the docking station 5 or other access point to which the wearable device 3 may be wirelessly connected or otherwise communicatively connected.

The first and second platforms 8 and 9 can each include various other network elements (e.g. routers, access points, gateways, border control devices, endpoints, etc.). In some embodiments, the first platform 8 can be a network that includes at least one wearable device data management device 7a. The first platform 8 can be operated by a distributor of the wearable device 3 in some embodiments or by a health care provider (e.g. hospital system, health care provider network, etc.). The second platform 9 can be a network that is operated by a health care provider that can include at least one electronic health record (EHR) database that includes a patient EHRs. The EHR database can be hosted by at least one patient data management device 7b (e.g. at least one server of a health care provider system). In other embodiments, the second platform 9 can be operated by the manufacturer and/or distributor of the wearable device 3.

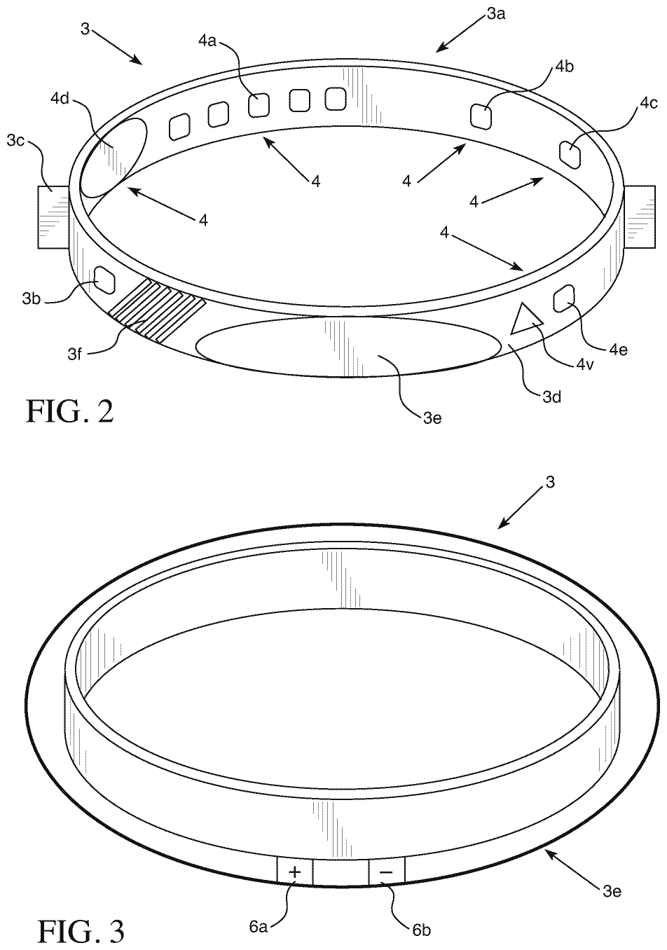

FIGS. 2-5 and 18-20 illustrate an exemplary embodiment of the wearable device 3 that can be utilized in the exemplary embodiment of the apparatus 1 shown in FIG. 1 and can be connectable to and/or positioned on a docking station 5 for recharging of the device's battery as well as to also facilitate collection and transmission of data to the central server 7 and/or other devices of the first platform 8 and. second platform 9. As may be best seen in FIGS. 1 and 20, the wearable device 3 can be configured to be positioned on or near the docking station 5 for connection to the charging unit of the docking station 5 so that the power source 3e of the wearable device can be charged when the wearable device is connected to the charging unit of the docking station 5 (e.g. positioned on the docking station to connect for wireless charging, positioned on the docking station and connected via a USB connection for charging of the power source, etc.).

In some embodiments, the docking station 5 can include a housing 5h that has an upper groove 5g and a lower groove 5g defined there to facilitate bands of the wearable device 3 being encircled about the housing of the docking station 5. The housing 5h can also include a centrally positioned annular light emitting device 5f that can be flashed or lit in a first color to indicate the power source 3e of the wearable device is being charged and illuminated in a solid continuous color of light or be illuminated in a second color that differs from the first color to indicate when charging of the power source 3e is completed. A controller within the housing 5h can be connected to the charging unit of the docking station, the light emitting device 5f, a wireless transceiver for receiving data from the wearable device 3 and sending that data to an input/output device 13 and/or central server 7, as well as other elements.

The wearable device 3 can include a sensor array 3a that can include a plurality of sensors 4 connected to a housing or frame of the wearable device 3. The sensors 4 of the sensor array 3a can be communicatively connected to the processor and/or computer readable medium of the wearable device as well. For example, the sensors 4 of the sensor array 3a can include: (i) at least one optical sensor 4a configured to detect a heart rate of a user and the content of the blood of the user wearing the wearable device 3 (e.g. oxygen content of blood, etc.), (ii) a temperature sensor 4b configured to measure the internal body temperature of the user wearing the wearable device 3, (iii) an accelerometer 4c configured to detect and/or measure movement of the wearable device 3 while the user wears the device to detect and measure movement by the user, (iv) a sweat sensor 4d configured to detect when a user wearing the wearable device 3 is sweating and measure (a) an amount of sweat or a rate of sweat being produced by the user, (b) a blood alcohol content of the user, and/or (c) a glucose level of the user, and (v) a microphone 4e configured to measure and/or detect sound emitted from the user for use in evaluating the user's breathing and/or other audible metric of the user while the user is wearing the wearable device 3. For example, the microphone 4e can detect and record user coughing or snoring that may occur when the user is detected as being asleep and also detect environmental conditions at night that can affect a user's ability to sleep (e.g. noise pollution environment, etc.). In some embodiments, the wearable device 3 can also include a vibration mechanism 4v. Embodiments of the vibration mechanism 4v can be configured so that when the vibration mechanism is actuated it causes the wearable device 3 or a portion of the wearable device 3 to vibrate.

The optical sensor 4a and/or other sensors 4 can also be configured to detect a user's glucose level, blood alcohol content of the user, and/or other metrics of interest. For example, the wearable device's sensor array 3a can also include a glucose monitoring sensor and/or a blood alcohol content sensor.

In some embodiments, the optical sensor 4a can include at least one light emitting diode (LED) and/or at least one optical detector. For example, the optical sensor 4a can include an optical detector, a red LED and an infrared (IR) LED. The red LED and the IR LED can each be configured to direct transmission of red light and IR light (i.e. electromagnetic radiation (EMR) with wavelengths longer than those of visible light within the IR spectrum) at a body of the user when the wearable device is worn by the user. The optical detector can be configured to measure a heart rate of a user based on measuring the red LED and infrared LED lights directed at the user's body as well as to detect whether the wearable device 3 is being worn by the user via the detection of the user's skin or heart rate. The optical sensor 4a can also include one or more green LEDs that can emit light to facilitate detection of the heart rate or other medical conditions.

The wearable device 3 can also include a flexible housing 3d that can retain the processor, computer readable medium, and sensors 4 of the sensor array 3a as well as other components of the wearable device (e.g. a power source, at least one antenna for wireless communications, at least one interface 3i, etc.). An adjuster 3c can be attached to the housing 3d or integrated into the housing 3d to permit the size of the annular structure of the wearable device 3 to be adjusted so that it can be worn by users having different sized body parts (e.g. arm, upper arm, lower arm, leg, upper leg, lower leg, chest, waist, wrist, neck, ankle, etc.). In some embodiments, the adjuster 3c can be configured as a band adjuster. In some embodiments, the adjuster 3c can include a plurality of magnetically connectable elements 3mce that are within portions of bands that are magnetically connectable to form an annular structure for being worn on a user's arm (e.g. around upper arm, lower arm, or wrist), chest, head, neck, leg (upper leg above knee or lower leg below knee), or ankle. The magnetically connectable elements 3mce can be partially or entirely encased within an outer covering of each strap of the wearable device or can be otherwise positioned on opposite straps of the wearable device for providing such functionality so that the straps can be connected together to form an annular structure after the bands are positioned around a user's wrist, arm, upper arm, lower arm, leg, lower leg, upper leg, ankle, chest, waist, or other body parts (e.g. head, neck, etc.).

The wearable device 3 can also include a wireless communication transceiver unit 3b positioned in the housing 3d or attached to the housing 3d. The wireless communication transceiver unit 3b can have an antenna that helps facilitate the transmission and receipt of data that is connected to the processor 3p. In some embodiments, the wireless communication transceiver unit 3b can be configured as a wireless interface for the transmission and receipt of data via a wireless connection with a docking station 5, input/output device 13, or an access point. For example, the wireless communication transceiver unit 3b can include a Bluetooth Low Energy (BLE) system-on-chip (SoC) module (also referred to as a “BLE SoC Module”, which is a type of SoC module) coupled to the circuit board 3g and the processor 3p of the wearable device 3 to facilitate Bluetooth wireless communications with the docking station 5, input/output device 13 and/or other communication devices of a local area network. In other embodiments, the wireless communication transceiver unit 3b can also include or alternatively include a Wi-Fi transceiver unit or other wireless communication transceiver unit (e.g. a near field communication transceiver unit, a wireless local area network transceiver unit, etc.).

The housing 3d of the wearable device 3 can also have or be attached to an identifier mechanism 3f In some embodiments, the identifier mechanism 3f can be a barcode, a matrix barcode (e.g. a quick response “QR” code), or a radio frequency identification tag (RFID tag) that is positioned on the housing (e.g. adhesively attached, integrally connected, defined thereon, etc.).

The housing 3d can also be connected to a rechargeable power source 3e. The rechargeable power source 3e can be positioned on an external surface of the housing or be positioned within the housing 3d. In some embodiments, the power source 3e can be configured as a thin film battery that is releaseably attached to the housing 3d (e.g. is attachable to and detachable from the housing 3d). The removability of the power source 3e can permit a new power source to be attached to the housing while an older power source 3e is recharged.

The power source 3e can include a negative terminal 6b and a positive terminal 6a. These terminals can be positioned on the housing 3d or within the housing 3d to facilitate connection to a recharging device of a docking station 5 for receipt of electrical current for recharging of the power source.

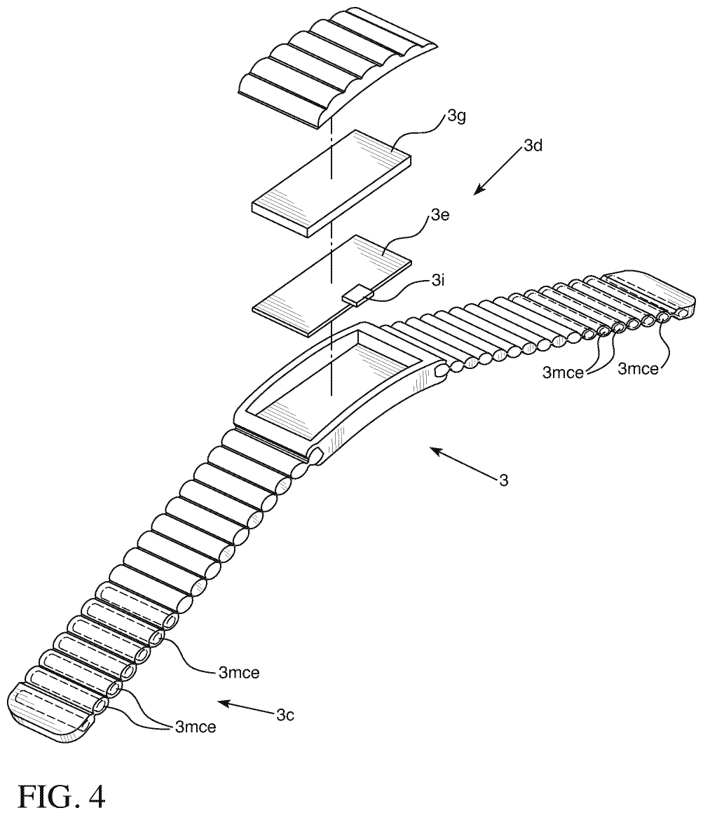

As may best be appreciated from FIG. 4, the wearable device 3 can include a rigid or flexible circuit board 3g positioned within the housing 3d. The circuit board 3g can have the processor, non-transitory computer readable medium, and other circuitry (e.g. wireless transceiver, etc.) positioned thereon. The power source 3e can also be positioned in contact with the circuit board 3g for powering the circuit board 3g. At least one interface 3i can be attached to the circuit board 3g and/or the power source 3e. For instance, the interface 3i can be configured as a USB interface to facilitate a wired connection to the circuit board 3g and/or power source 3e for the transmission of data and/or electrical current to the wearable device 3 as well as permitting the wearable device 3 to transmit data to another device (e.g. docking station 5, central server 7, etc.) via the interface 3i. A liquid crystal display 13a or other types of display 13a can also be attached to the housing and connected to the processor 3p and the non-transitory computer readable medium of the wearable device 3. The display 13a can be configured to be detachable or deactivatable to reduce power consumption.

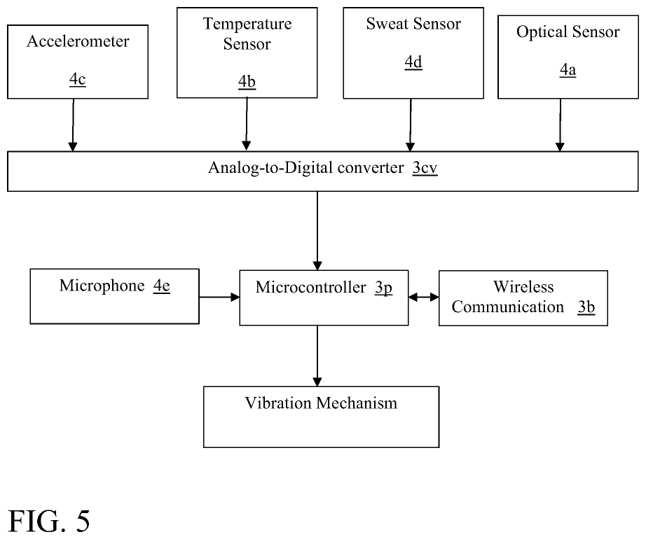

FIG. 5 illustrates a schematic view of components of the exemplary wearable device shown in FIGS. 1-4. As may best be appreciated from FIG. 5, the circuit board 3g can include an analog-to-digital converter 3cv that connects at least some of the sensors 4 of the sensor array 3a to the processor 3p. For example, measurement data or other data sensed or detected by the accelerometer 4c, temperature sensor 4b, sweat sensor 4d, and optical sensor 4a can be recorded as analog data and sent to the memory of the device for storage in the memory. The analog-to-digital converter 3cv can receive the data before it is stored in the memory to convert the analog data to digital data in embodiments in which the sensor data is analog data.

Other sensors of the sensor array 3a can be communicatively connected to the computer readable medium and/or the processor 3p without the use of a converter. For example, the microphone 4e can be configured as a digital microphone that can be connected to the computer readable medium and/or processor 3p so that the audible data collected by the microphone is stored and/or otherwise used without being converted via an analog-to-digital converter.

The processor 3p can be connected to a wireless transceiver unit 3b and/or the interface 3i so that sensor data received from the sensors (e.g. directly from sensors and/or via at least one converter 3cv) can be transmitted to the docking station 5, a central server 7, input/output device 13, or other computer device for longer-term storage and evaluation for detection of a user health condition and/or monitoring of a treatment being provided to the user. As discussed herein, the input/output device 13, docking station 5, and/or central server 7 can be configured to utilize the sensor data received from the wearable device 3 for storage and also use that data to perform calculations and other evaluations, as well as provide data for generation of a graph, chart, text, or other display of information via a GUI for providing information to a user about the user's detected sleep via a display 13a.

In some embodiments, the sensor data can also be stored within the non-transitory computer readable medium of the wearable device 3 for an extended period of time and the processor 3p can be configured to evaluate the stored data to detect a condition of the user and/or monitor a treatment being administered to the user. The wearable device 3 can also be configured to utilize stored data to perform user evaluations of that data or emit output as discussed herein, for example.

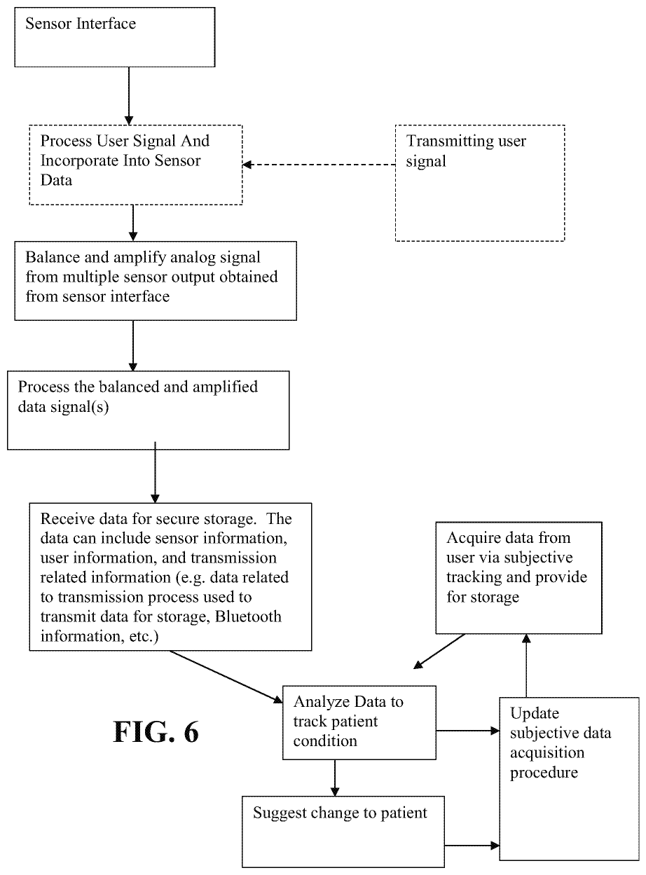

FIG. 6 illustrates an exemplary method by which the sensor data collected by the sensors 4 of the sensor array 3a can be processed for storage and/or transmission. For instance, the sensor data can be obtained from sensors 4 of the sensory array 3a via at least one sensor interface that connects the sensors to the processor 3p. The sensor interface can include at least one analog-to-digital converter 3cv in some embodiments. The sensor data can be processed via the sensor interface (e.g. converted or otherwise processed). For example, the sensor data can be balanced and amplified before being subsequently processed by a converter 3cv. The converted data can then be transmitted for secure storage and/or stored on the computer readable medium of the wearable device 3. When the stored sensor data is transmitted to the docking station 5, input/output device 13 and/or central server 7, transmission related data can be included with the transmitted sensor data to identify the transmission process used to transmit the sensor data, time of transmission, time at which sensor data was stored and/or collected, sensor device identifier for each type of sensor data, as well as other data relevant to the collection and storage of the sensor data for subsequent evaluation by the wearable device 3, central server 7, input/output device 13, docking station 5, and/or other computer device.

In some embodiments, the processor can run an application that defines a method by which the microphone 4e can be utilized to collect user data related to the user's snoring or coughing that can occur while the user is asleep. For instance, the microphone can be activated to record audible noise upon the processor determining the user is asleep via heart rate and/or accelerometer data. The processor 3p can store data for each cough event and each snoring event that is recorded along with time data to identify the duration of the cough event and/or snoring event that is detected by the microphone 4e. This data can be stored and subsequently transmitted to the docking station 5, input/output device 13, and/or central server 7 for subsequent evaluation to detect coughing or snoring pattern of the user and correlate that pattern with a particular health condition related to the user's sleep (good sleep, insomnia, etc.).

As can be seen in FIG. 6, the wearable device 3 or another aspect of the apparatus (e.g. a central server, a server communicatively connected to a smart speaker or other types of input/output device 13, docking station 5 connected to an input/output device 13, or an input/output device 13 such as a smart speaker, touch screen display 13a of a tablet or smart phone, or health monitoring device having a speaker, a microphone, a touch screen display 13a, a keyboard, and/or a pointer device connected to a processor running at least one application stored in the memory of the device to facilitate receipt of user input while also providing visual and/or audible output to a patient, etc.) can also be configured to acquire subjective data from a patient via output that can be provided audibly and/or visually (e.g. text, a video, audible prompts or questions, etc.).

The subjective data acquisition can include querying the patient about food the patient may have consumed at a particular time, how the patient feels after waking up from sleeping, or other questions or prompts to receive input from the patient related to such information. Such information can be classified as subjective information and be stored for use in analyzing the patient data along with the more objective data acquired via the wearable device 3. The subjective and objective data can be analyzed and the patient can subsequently be provided output to suggest a behavioral change to try and improve the patient's sleep quality, sleep duration, or other health condition. The querying utilized to acquire subjective data input from a patient can also be updated after such behavioral changes are suggested and/or as a result of the analysis of data related to the monitoring of a patient's condition or treatment so that additional or alternative information can be acquired from the patient via the subjective data querying. The wearable device 3, input/output device 13, docking station 5, and/or central server 7 can be configured to utilize the sensor data and facilitate the acquisition of the patient's subjective data. as well as provide data for generation of a graph, chart, text, or other display of information via a GUI for providing information to a user to receiving subjective data from the user or other data for storage and subsequent use in the evaluation of at least one condition of the patient (also referred to herein as the user).

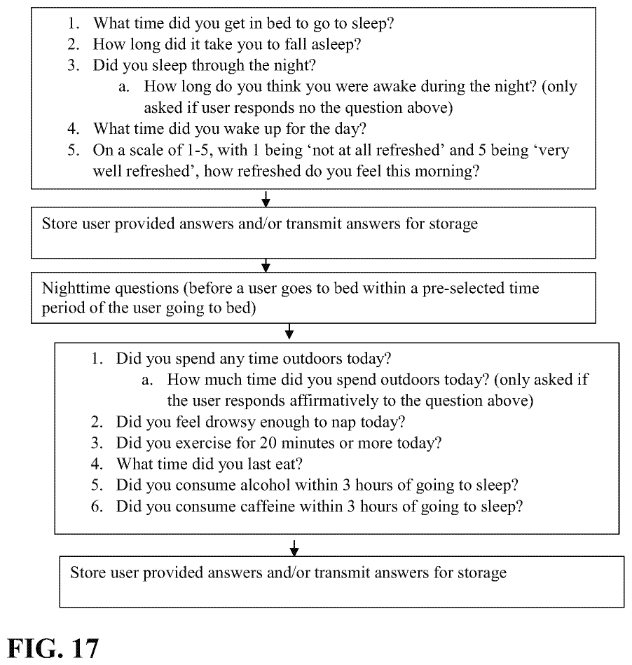

FIG. 17 illustrates an example of questions that may be asked via output provided to a user to solicit receipt of subjective data input the user may provide via at least one input device (e.g. a microphone, a keyboard, a touch screen display, a pointer device, a mouse, a combination of these input devices, etc.). The questions can be provided audibly via a speaker and/or maybe provided visually via a display of text provided by a graphical user interface that is generated on a display. For example, a user can be asked questions within a pre-selected time period before the user is to go to bed. This time can be determined so that the user is automatically prompted for providing this input via queries at a particular time via a smart speaker, smart phone, or other electronic device that the user may have linked to his or her account with the system. Alternatively, the user can activate an application for having these questions asked for providing this input at a user selected time before going to bed and/or after waking up from sleeping.

For instance, after waking up, the user can be asked the following questions:
1. What time did you get in bed to go to sleep?
2. How long did it take you to fall asleep?
3. Did you sleep through the night?
   a. How long do you think you were awake during the night? (only asked if user responds no the question above)
4. What time did you wake up for the day?
5. On a scale of 1-5, with 1 being ‘not at all refreshed’ and 5 being ‘very well refreshed’, how refreshed do you feel this morning?

The user's input responsive to each of these questions can be stored in connection with date and time information so that the user's input can be stored and used in the subsequent analysis for the generation of different charts, graphs, other types of displays and for analyzing different sleep patterns and behaviors that can be linked or associated with those sleep patterns. The wearable device 3, input/output device 13, docking station 5, or central server 7 can be configured to utilize the sensor data and the acquired subjective data and perform this analysis as well as provide data for the generation of a graph, chart, text, or other display of information via a GUI for providing information to a user via a display 13a.

As another example, after waking up, the user can be asked the following questions:
1. Did you spend any time outdoors today?
   a. How much time did you spend outdoors today? (only asked if the user responds affirmatively to the question above)
2. Did you feel drowsy enough to nap today?
3. Did you exercise for 20 minutes or more today?
4. What time did you last eat?
5. Did you consume alcohol within 3 hours of going to sleep?
6. Did you consume caffeine within 3 hours of going to sleep?

The user's input responsive to each of these questions can be stored in connection with date and time information so that the user's input can be stored and used in the subsequent analysis for the generation of different charts, graphs, other types of displays and for analyzing different sleep patterns and behaviors that can be linked or associated with those sleep patterns. The wearable device 3, input/output device 13, docking station 5, or central server 7 can be configured to utilize the sensor data and the acquired subjective data and perform this analysis as well as provide data for the generation of a graph, chart, text, or other display of information via a GUI for providing information to a user via a display 13a.

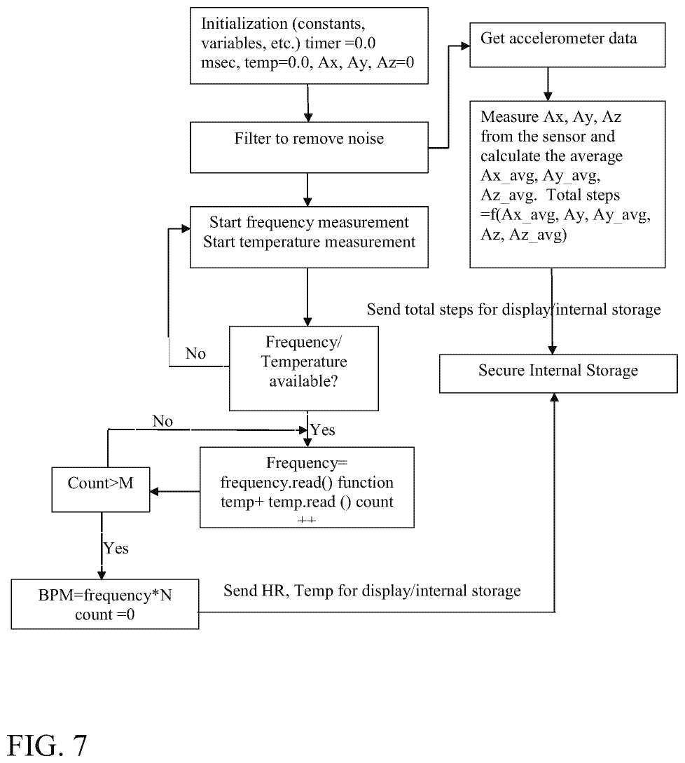

FIG. 7 illustrates an exemplary process by which sensor data can be collected and subsequently processed for storage via sensors 4 of the sensor array 3a connected to the processor 3p. For example, temperature sensor data and optical sensor data can be received over a pre-selected time period for use in measuring at least one health metric of the user. That received sensor data can be filtered to remove noise. To evaluate the heart rate, the temperature and optical sensor data can be received periodically for a pre-selected time period, or frequency to determine a heart rate (e.g. beats per minute (“BPM”), etc.). The heart rate data and the body temperature data can be stored in the non-transitory computer readable medium for storage and subsequent transmission to the first platform 8 or the second platform 9 (e.g. central server 7 via docking station 5, directly from the wearable device 3 to the central server 7 via a wireless access point or cellular network base station, etc.) to the docking station 5 for subsequent processing. The data can also be processed for display on the display device of the wearable device.

Other sensor data can also be collected and processed for storage. For example, as shown in FIG. 7, accelerometer data can be received from the accelerometer 4c and that measurement data can be processed to determine the total number of steps the user has taken in a pre-selected time period (e.g. number of steps taken in a day, number of steps taken per hour, etc.). That measurement data can be processed for storage and/or display as well.

The wearable device 3 can also utilize other methodologies for detecting different health conditions that may be related to sleep. For example, the accelerometer and heart rate data can be processed and evaluated to determine whether the user is sleeping or attempting to sleep. Such data can be evaluated in the context of time information that is maintained by a timer (indicating an amount of time the user has not moved beyond a movement threshold for sleep detection or attempted sleep detection) and/or clock information (e.g. identifying the time of day and date)) of the wearable device 3. In response to determining that the user is attempting to sleep or is asleep, the microphone can be activated for recording sound emitted by the user or in the environment around the user to evaluate environmental noise, user coughing, user snoring, and other sound issues that may affect the user's sleep. That recorded data can be stored continuously or can be stored so that only sounds that exceed a pre-determined threshold are stored. The stored sound data can be stored so it is associated with the clock information and/or timer information indicating a time and date at which the data was recorded and stored. The stored data can also be linked or associated with other stored data, such as a duration at which the user was detected as being asleep or attempting to go to sleep via other sensor data. This collected and stored data can then be processed to evaluate the quality of the user's sleep, whether the user had an insomnia event, or other issues. The wearable device 3, input/output device 13, docking station 5, or central server 7 can be configured to utilize the sensor data and perform this calculation. as well as provide data for the generation of a graph, chart, text, or other display of information via a GUI for providing information to a user about the user's detected sleep via a display 13a.

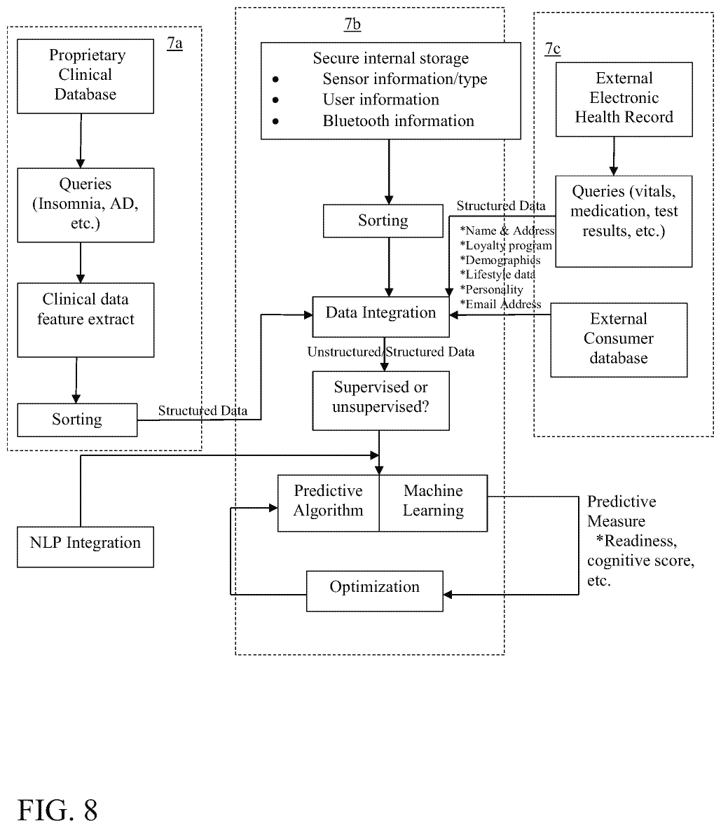

FIG. 8 illustrates an exemplary process by which data obtained via the sensor array 3a of the wearable device 3 can be utilized by the wearable device 3 as well as (or alternatively) the central server 7, docking station 5, input/output device 13 or other computer device for evaluating the sensor data for use in monitoring a health condition of a patient and/or treatment provided to a patient.

The sensor data from the wearable device 3 can be received at the central server 7 as shown in FIG. 8 (or the docking station 5 in some alternative embodiments). The received sensor data can be sorted or otherwise processed by the central server for forwarding to the wearable device data management device 7a and/or the patient data management device 7b. The central server 7 can also be communicatively connected to these data management devices so that data stored in those devices can be retrieved by the central server 7 for use by the central server 7 (in addition to the central server 7 being able to send data to those devices for storage).

The wearable device data management device 7a can have non-transitory memory for storing data from many wearable devices of different users. The data can be stored so that the data can be stored in at least one large database. The data from each wearable device 3 can be stored so that the data is sortable based on a number of different metrics. These metrics can include wearable device location, type of treatment the wearable device is to monitor, type of condition detected via the wearable devices, etc. This data can be stored so it can be queried and sorted in a number of ways to facilitate the evaluation of a large population of data from a large number of wearable devices so that the data can be evaluated to assess how a particular type of condition is being addressed by a particular type of treatment for a large population of users. Such data collection and evaluation can facilitate clinical research activities by facilitating the evaluation of how a placebo group monitored by wearable devices progressed as compared to a group that was given a particular pharmacological based treatment for the same condition.

The collection of data provided by the wearable devices 3 can also permit such data to be addressed at a group level and also at an individual level to allow for a more granular evaluation of data that can better account for a particular patient's specific health metrics while also permitting evaluations at a larger group specific level so that sub-groups of interest can be better evaluated to account for unanticipated reactions to a particular treatment or other circumstances that may arise from a given treatment to allow for a more effective and efficient evaluation of the treatment and its potential side effects.

For example, microphone data from the wearable devices can be stored and subsequently evaluated to identify coughing and/or snoring patterns that can be associated with an insomnia condition or other condition affecting a user's sleep quality. Such data can also be linked with other user data (e.g. blood alcohol content, glucose level, environmental noise levels, etc.) to better predict a bad sleep condition or an insomnia triggering event. For instance, the data can be processed so that a prediction can be made in response to determining an association with a particular pattern may exist with the bad sleep condition and/or an insomnia triggering event to better predict the bad sleep condition or an insomnia triggering condition.

The patient data management device 7b can receive wearable device data from the central server 7 for different users for storage so that the user's data is stored with other health metric data of the user stored in the patient data management device 7b (e.g. lab report data, other health metric data of the user from procedures or hospital visits, etc.). The patient data management device 7b can be configured to store the user's wearable device 3 data so it is linked and associated to the user of the device and stored with the user's other health data (e.g. data related to medication, vitals taken of the user at different times by health care providers, lab work results, personality testing results, mental health related care data, etc.) so it can be queried and sorted. The data can also be linked to other data specific to the user such as the user's name, address, contact information, and demographic information in the patient data management device 7b. The central server 7 can be configured to communicate with the patient data management device 7b to obtain such data. In other embodiments, the central server 7 can be connected to the patient data management device 7b so that only HIPPA compliant data related to the user stored on the patient management device can be obtained and evaluated by the central server 7.

The central server 7 can be configured to integrate data from the patient data management device 7b and the wearable device data management device 7a for evaluation of that data. The evaluation of the data can be performed to evaluate the efficacy of one or more treatments provided to users of the wearable devices in some embodiments. The central server 7 can be configured to evaluate such data automatically in accordance with a pre-defined method that is defined by one or more applications that may be stored on the memory of the central server 7. The central server can also (or alternatively) help evaluate such data by running one or more applications in conjunction with the receipt of input provided by a user of the central server via at least one input device (e.g. keyboard, pointer device, mouse, stylus, touch screen display, etc.) that may be provided by use of a graphical user interface supported by the central server 7. The central server 7 can be configured to natural language processing (NLP) for use in assessing and reacting to user input and may also utilize machine learning and predictive algorithms to optimize data processing it may perform. The evaluation of the stored data can permit the large collection of data from a large number of users to detect triggers for insomnia or other sleep related conditions a user may experience that could require an intervention. The collected and stored data can also be used to compare and contrast how large groups of users react to a particular intervention (e.g. a behavioral change, taking a particular medication, taking a particular dose of a drug, etc.) by comparing their pre-intervention data with their data collected while utilizing or engaged in the intervention.

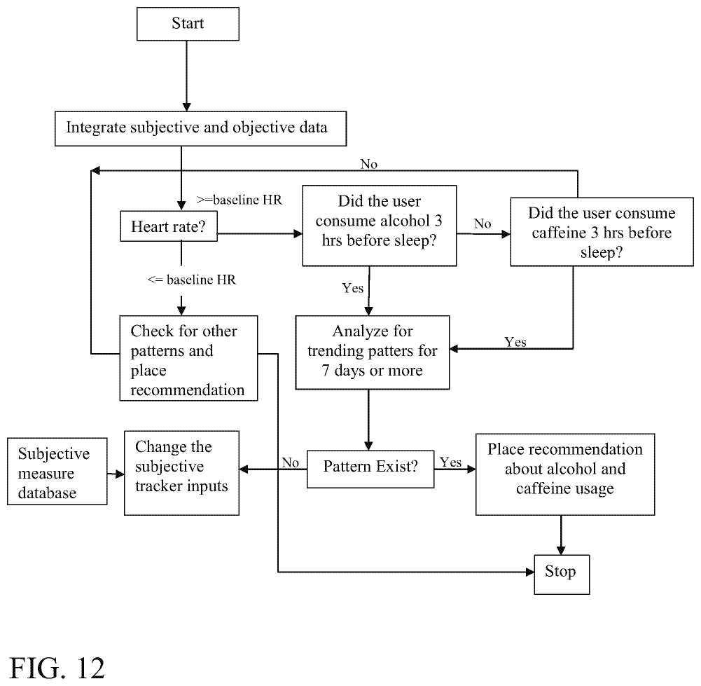

The data that is collected can also be utilized at a user-specific level to evaluate the type of sleep a user is experiencing and to predict different user activities that can trigger good sleep or trigger insomnia or other sleep related problems. Some examples of data collection and processing that can be utilized using embodiments of the wearable device 3 and apparatus 1 can be appreciated from the exemplary processes shown in FIGS. 12-17, 21-23, 25, and 27.

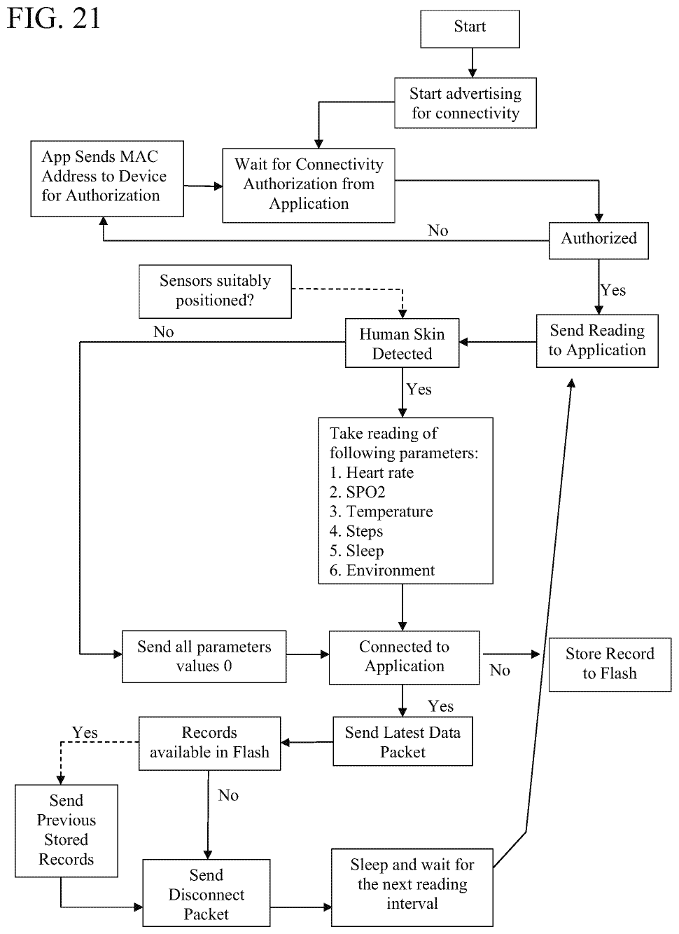

As may best be appreciated from FIG. 21, the wearable device 3 can be configured to facilitate connections to one or more input/output devices 13 and/or docking station 5 in response to activation of the wearable device 3. The wearable device 3 can have a power button defined on the housing or have another mechanism for being activated (e.g. detection of being in contact with skin to automatically turn on the device, etc.). In response to being activated, the wireless transceiver unit can output a transmission to other devices to advertise its availability for connection for forming a wireless communication connection with a desired input/output device 13 and/or the docking station 5. The input/output device 13 and/or docking station 5 may have an application stored in their memory that can be executed by a processor of that device to facilitate automatic connection with the wearable device in response to the connectivity advertisement transmission output from the wearable device 3.

In an event where the wearable device 3 had not previously formed a secure authorized connection, it may respond to the availability of the input/output device 13 and/or docking station 5 by requesting and/or receiving the MAC address of the device to which the wearable device 3 is to connect for establishing the communicative connection with that device. Additional protocols may be utilized to form such a connection. In addition, a user may have to enter a password or other information into the input/output device 13 or docking station 5 to facilitate the formation of the connection with the wearable device by use of an application stored thereon that is run to facilitate connection with the wearable device 3 and/or receipt of data from the wearable device 3.

After an authorized connection is formed with the docking station 5 and/or input/output device 13, the wearable device 3 can be configured to send stored sensor data collected from the user when the user wears the device to the input/output device 13 and/or docking station 5. This data may subsequently be sent to the central server 7 that can be connected to the input/output device 13 and/or docking station 5 (e.g. via a network connection and application programming interface (API) the server may have with the input/output device running the application, etc.). The central server 7 may also send this received data or a portion of it to the wearable device data management device 7a and/or patient data management device 7b for organization and storage by those devices.

The wearable device 3 can be configured to communicate at least one challenge to any device to which it is connectable to verify that the input/output device 13 and/or docking station is authorized to receive data from the wearable device 3. The particular protocol utilized for forming a connection or pairing the wearable device 3 to the docking station 5 and/or one or more input/output devices 13 can be adjusted to meet any particular type of desired communication protocol and authorization protocol that may be desired to account for ease of use and a desired level of security.

After a connection to at least one authorized device is formed, the wearable device 3 can send sensor data that has been collected and saved to the input/output device 13 and/or docking station 5 since the last time such data was transmitted to that device. In some embodiments, the wearable device 3 and input/output device 13 or docking station 5 can perform a synchronization to ensure that the data that the wearable device has provided includes all new data that the connected device has not yet received from the wearable device 3 since they were last communicatively connected to each other.

While connected to at least one paired device (e.g. input/output device 13 and/or docking station 5), the wearable device 3 can also be activated for further collection of data from a user in response to determining that it is being worn by the user. Such data collection can be actuated by the wearable device determining that it is positioned on human skin, for example. The wearable device 3 can be configured to make this determination automatically without requiring any input from a user who may be wearing the device.

If the wearable device 3 determines it is not currently being worn and has confirmed it has previously successfully transmitted its sensor data to at least one other device (e.g. input/output device 13 or docking station 5) for transmission to the central server and/or that device, then the wearable device 3 can clear its saved data and set relevant parameters to zero or other default position for subsequent data collection. The clearing of saved data can be performed to only delete certain old data while maintaining other more recent data as an alternative to clearing all saved sensor data that the wearable device 3 has communicated to at least one other device. In yet other embodiments, no such clearing may occur automatically and may only occur in response to receipt of input instructing the wearable device 3 to delete some or all of the saved data that may be communicated to the wearable device 3 via the input/output device 13 or docking station 5.

In response to determining that the wearable device 3 is being worn by a user, the device can activate its sensor array to activate the sensors for monitoring one or more health conditions of the user. The monitoring may be performed continuously or at pre-selected monitoring intervals (e.g. every five seconds, every minute, etc.). Each sensor of the sensor array may collect data continuously or discretely within pre-selected sensor measurement intervals spaced apart from each other by a pre-selected monitoring interval time period.

If the wearable device 3 is connected to the input/output device 13 or docking station 5, the sensor data collected by the sensors can be sent immediately to the device(s) to which it is connected and can also save that data in its own memory 3m as well. If no such connection is formed, the collected data can be saved in memory 3m for subsequently sending it after such a communication connection with the input/output device 13 or docking station 5 is subsequently formed.

In the event there is a communication error detected that resulted in some transmitted data not being received by the input/output device 13 or docking station 5, the wearable device 3 can be configured to detect the error and resend that data. For instance, the wearable device 3 can detect that it lost its Bluetooth connection for a period of time with a paired device before then re-establishing that connection (e.g. due to device power failure, the device being outside the range of the transceiver unit 3b, etc.) and then resend the affected data after that connection is restored.

After collected and stored sensor data has been sent to the device to which it is paired or otherwise communicatively connected, the wearable device 3 may stop transmitting sensor data for a pre-selected period of time to conserve the power of the device. For example, after transmission of the sensor data, the wearable device 3 can halt such transmissions and store sensor data for at least a pre-selected transmission interval time period. After this time period elapses, the wearable device 3 can then send the newly collected and not yet previously transmitted sensor data to the input/output device 13 and/or docking station 5 to which the wearable device 3 is paired or communicatively connected. This communication of sensor data may occur repeatedly after every pre-selected transmission interval time period may pass while the wearable device 3 remains connected to and paired with the input/output device 13 and/or docking station 5. The pre-selected transmission interval time period can be a time period that is deemed to be suitable for a particular set of design criteria. It can be 30 seconds, a minute, five minutes, ten minutes, less than 30 minutes, not more than 30 minutes, or another time period that is between 1 second and 30 minutes in some embodiments.

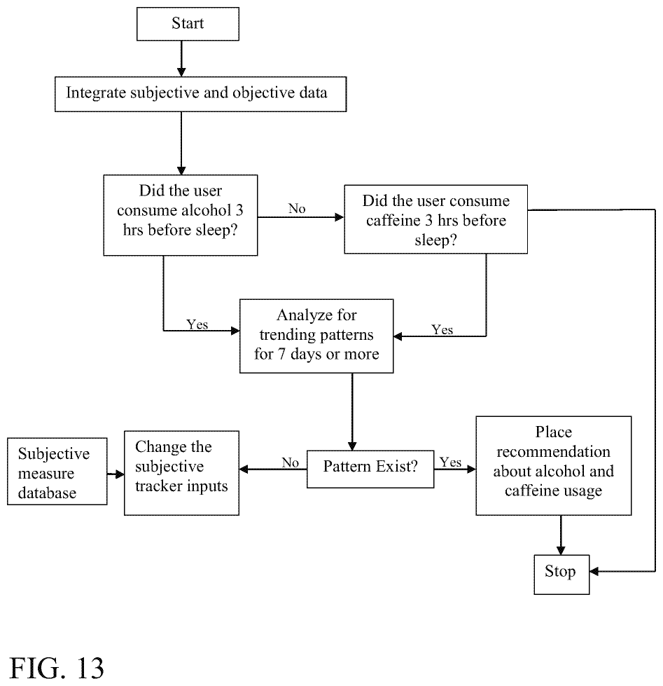

With reference to FIGS. 12 and 13, objective data obtained via the array 3a of sensors 4 can be stored in a memory of an input/output device 13 or a central server 7. Additionally, subjective data obtained via at least one input/output device 13 can be collected and stored in the memory of the input/output device 13 and also forwarded on to a central server 7 for analysis and use. The subjective data can be data acquired from the user that requests the user to provide input for storage of data that can be relevant to a condition of the user being monitored. Such information that is requested can include solicitation of yes or no inputs on questions related to consumption of food or drugs (e.g. consumption of alcohol, consumption of caffeine, consumption of marijuana) or other activity (patient activity levels within the day or an extent to which the patient performed physical exercise, etc.). The subjective data can be obtained from a patient via at least one input/output device 13, such as a smart speaker, tablet, or smart phone that can run an application that defines a methodology of acquiring the subjective data from the user for storage in a central server that can be connected to the input/output device 13 via a network connection or other type of communicative connection. In some embodiments, the central server 7 may host services in conjunction with the application being run on the input/output device 13 for subjective data acquisition and storage. The central server 7 can receive the user data via an API connection or other connection the central server 7 has with the input/output device 13 for storing that data for use in providing the service to the user or the input/output device 13.

For example, the subjective data that is obtained can include the input/output device 13 querying a patient about whether the patient has consumed alcohol within a first pre-sleep time period before the patient went to bed and/or whether the patient has consumed caffeine first pre-sleep time period before the patent went to bed. The patient's input provided in response to such queries can be provided to a central server 7 for storage. The central server 7 can also receive objective patient data via the wearable device 3 as discussed herein. The central server 7 can store the patient's subjective data and objective data so that the data can be integrated and analyzed for identifying patient patterns of behavior and health conditions for different times within a time period (e.g. different times of a day, week, month, year, and/or number of years).

For example, after the subjective and objective patient data is stored and integrated, the patient's heart rate data can be evaluated during a time period at which the patient was detected as being asleep. If the heat rate is less than a pre-selected heart rate limit (e.g. 70 beats per minute, 80 beats per minute, etc.) then the patient's heart rate may be considered to be within a normal or healthy range and other objective data of the patient can be evaluated to detect possible patterns of behavior related to the patient's ability to sleep, duration of sleep, and/or quality of sleep.

If the patient's heart rate is over the pre-selected heart rate limit, the patient can be determined to potentially have a condition that could affect the patient's sleep or other health related condition. In response to such a determination, the subjective data of the patient can be further evaluated to determine if potential patterns of conduct of the patient may contribute to a heart rate condition that may detrimentally affect the patient's sleep. For example, subjective data input provided by the patient to an input/output device concerning whether the patient consumed alcohol within a pre-selected alcohol consumption time limit (e.g.one hour, three hours, five hours, 24 hours, 48 hours, etc.) can be evaluated. If the patient has not had any consumption of alcohol within the pre-selected alcohol consumption time limit, another subjective factor can be evaluated. For example, the subjective patient data can be evaluated to determine if the patient had consumed caffeine within a pre-selected caffeine consumption time limit (e.g. one hour, three hours, five hours, 24 hours, 48 hours, etc.). Other subjective topics related to food or drug conditions could also be evaluated in addition (or as an alternative) to caffeine and alcohol (see e.g. FIG. 17).

In response to the patient's subjective data indicating that no possibly problematic food or drug consumption occurred, other patient data can be evaluated to try and detect a pattern that could have affected the patient heart rate metric. This additional information could include, for example, accelerometer data or other subjective data for evaluating whether the patient underwent significant physical activity prior to attempting to sleep, for example.

In response to the patient's subjective data indicating that there may be a problematic food or drug consumption that occurred, the patient data can be further evaluated to detect a pattern of behavior in which an elevated heart rate is associated with patient food or drug consumption that was identified. The detection for a pattern of behavior can include an evaluation of data that extends over a pre-selected pattern of behavior evaluation time period (e.g. seven days, two weeks, a month, etc.). If the pattern of behavior is detected, the central server 7 can communicate with the wearable device 3 and/or the input/output device 13 to provide a recommendation output to the patient to suggest a change in food or drug consumption prior to sleeping. For instance, if the patient had consumed alcohol or caffeine within a pre-selected consumption time period, the patient can be provided with output indicating that this consumption could be problematic and that the patient should attempt to avoid such consumption for a pre-selected remediation time period to try and evaluate whether that change can improve the patient's sleep quality and/or sleep duration. This type of output can be communicated to provide an audible, video or text based suggestion to the patient via a GUI displayed on a display of the wearable device 3 or a display of the input/output device 13.

As yet other examples, a determination that the patient did not exercise sufficiently or that the patient was not exposed to a pre-selected duration of sunlight based on the patient's subjective data inputs can result in the system outputting a suggestion for the patient to exercise more, exercise more outside, or do other tasks to increase the duration of exercise and/or exposure to natural sunlight.

Further, the output that is provided to suggest a behavioral change to the patient can be configured to include a graphical display that may further illustrate the detected pattern. For instance, a graph illustrating the patient's sleep quality and/or sleep duration in association with the patient's drug or food consumption can be shown to help explain to the patient why the suggested change in behavior has been recommended. The evaluation of the data and the output of the suggestion can occur automatically so that no user input is needed to evaluate the data and provide the output to the patient. In some embodiments, the suggestion and data evaluation can occur automatically after the patient provides a first input to trigger initialization of the evaluation of the data via the input/output device 13 or the wearable device 3. The input can be communicated to the central server 7 for the central server to process the data and communicate with the wearable device 3 or input/output device 13 for providing the output to the patient related to the evaluation of the patient data and recommendation of a change in patient behavior.

After a suggested change is made, the user can be prompted to provide a first input indicating whether the patient will try and implement the change. Such input can be utilized to update subjective tracker data for follow-up communications with the patient to prompt the patient to provide input in response to other subjective data queries related to food consumption, drug consumption and/or patient behavior for further evaluation of the patient's sleep that may occur after the patient tries to implement the change in behavior that was suggested. Such data that is acquired and stored can subsequently be utilized in a later evaluation of the patient data that can be performed for a second pre-selected pattern of behavior evaluation time period that may run from a time after the patient indicated the behavioral change would be made up to a time that the patient's condition was then re-evaluated to assess how effective that behavioral change was to the patient's sleep.

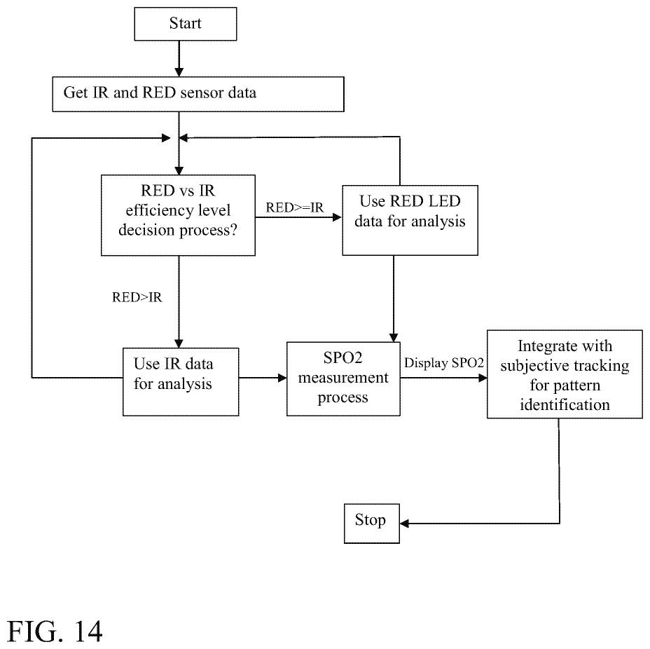

The triggering of a patient data evaluation of patient sleep condition evaluation, such as the evaluation that integrates subjective and objective data shown in FIGS. 12 and 13 and discussed herein can be triggered in different ways. For instance, as discussed herein, user input can be provided via a user interface run on an input/output device 13 (e.g. smart phone, smart speaker, computer, tablet, etc.) or wearable device 3 or other devices to trigger a central server 7 that hosts a service to perform the evaluation based on the user subjective and objective data that has been stored and integrated. Such an evaluation can also be triggered due to an evaluation of objective data that may have been performed at a pre-selected time by the central server without any particular user input provided trigger. One such example of this type of automated evaluation triggering process is shown in FIG. 14.

As can be appreciated from FIG. 14, the wearable device 3 can be configured to collect data from an IR and red light LED sensors of the array of sensors 4. The wearable device can be configured to determine which of the IR or red light LED sensor should be used for determining a peripheral capillary oxygen saturation value, SPO₂, (e.g. an estimate of the amount of oxygen in the blood of the patient wearing the wearable device 3). For instance, if the red light LED sensor provides a greater than or equal efficiency of evaluating the patient's SPO₂, the red light LED sensor data can be used. But, if the IR sensor provides a greater efficiency level for measurement and/or evaluation of the SPO₂, then the IR sensor data can alternatively be used. After selecting which sensor to use, that sensor's data can then be used to evaluate SPO₂ of the patient to determine an SPO₂ value. The wearable device 3 can communicate the SPO₂ value for display on a display of the wearable device 3, a display of the docking station 5, a display of an input/output device 13, or display on a user interface that is displayable to a user via a central server 7. The SPO₂ value data can also be stored and transmitted for use in integrating subjective and objective patient data for monitoring and/or evaluation of a condition of the patient (e.g. sleep quality and/or sleep duration of the patient, etc.).

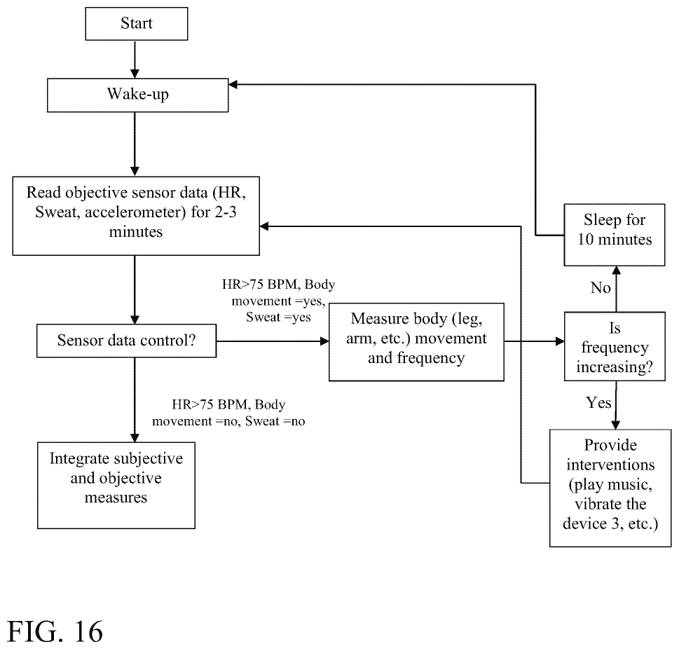

The wearable device 3 can also be configured to facilitate a treatment that can be provided to a patient wearing the device while the patient is asleep. Such a treatment can occur automatically via the wearable device 3 while the patient is still asleep. FIG. 16 illustrates an exemplary embodiment of such a process.

For example, the wearable device 3 can be configured to activate one or more of its sensors 4 of its sensor array at a pre-selected time interval for periodic measurement collection that can be used to trigger an automatic response to try and improve the quality and/or duration of the patient's sleep while also monitoring the patient's sleep. For at least some of the sensors 4, the wearable device 3 can be configured so that the sensors are continuously collecting data from the patient. In other embodiments, all the sensors 4 can be activated at different pre-selected time intervals for the collection of measurement data of the patient while the patient is wearing the wearable device 3.

The wearable device 3 can be configured so that different sensor data is received and evaluated for a pre-selected evaluation time period (e.g. a time period of 1 minute, 2-3 minutes, 5 minutes, etc.). The sensor data can include heart rate sensor data, sweat sensor data, and accelerometer sensor data. The wearable device 3 can be configured to evaluate the sensor data to determine whether the patient has measurement data that exceeds a pre-selected movement frequency threshold or pre-selected poor sleep quality threshold. Such a threshold can include a collection of parameters. For example, if the patient heart rate is over a heart rate baseline (e.g. 75 beats per minute, etc.), and the patient is also detected as sweating and having accelerometer data indicating the patient has been moving, the wearable device 3 can be configured to detect a possible problematic sleep condition and, as a result, further evaluate the patient objective measurement data for a pre-selected period of time to determine if the patient is experiencing an increase in body movement, sweat, and/or relatively high sleeping heart rate. The wearable device 3, input/output device 13, central server 7, or docking station 5 can be configured to evaluate the user data, or patient data, as well after that data is received from the wearable device 3 for use in the evaluation of the patient's sleep related data for use in determining different sleep quality parameters (duration of deep sleep, REM sleep, light sleep, etc.) and evaluation of the user's sleep.

If the frequency of sleep movement type events is increasing and/or the body movement, heart rate, and sweat data of the patient exceed a pre-selected sleep problem threshold, the wearable device 3 can be configured to communicate with at least one input/output device 13 and/or the docking station 5 or central server 7 to cause at least one output to be emitted while the patient is sleeping. For example, the wearable device 3 can be configured to vibrate via a vibration mechanism to provide a sensory and/or audible output that may help reduce patient movement, sweat, or heart rate to try and improve the quality of sleep the patient is experiencing. The vibration mechanism can be configured as a vibration motor, a shaftless vibration motor, a linear resonant actuator, a vibration motor that produces an oscillating force across a single axis, or an eccentric rotating mass (ERM) motor that is connected to the wearable device housing. The vibration mechanism can be configured so that when the vibration mechanism is actuated, the housing of the wearable device or the entire wearable device 3 can vibrate. In addition, the wearable device 3, input/output device 13 or docking station 5 can output an audible output via a speaker by playing a particular type of music or sound that the patient can hear while asleep to try and improve the sleep duration and/or sleep quality of the patient.

In the event the frequency of the patient movement, sweat, and heart rate is not increasing and these values are not above a pre-selected activation threshold, the wearable device 3 can be configured to stop collecting sleep data of the patient for a pre-selected period of time to preserve the battery life of the wearable device 3. After that pre-selected delay period of time is reached, the wearable device 3 can re-activate the sensors 4 for collection and evaluation of the data to evaluate whether the patient is experiencing a sleep related issue while asleep.

The wearable device 3 can also be configured so that the patient data that is collected while the patient is asleep is periodically transmitted to the docking station 5, input/output device 13, and/or central server 7 for storage and further analysis or evaluation. Such periodic transmission can occur while the patient is asleep and the sensors are activated to collect the patient data after each such activation sequence and can also include data identifying the time that an output was provided to the sleeping patient and what type of output was provided to the sleeping patient (e.g. just vibration, vibration and music via a speaker of the wearable device 3 or a speaker of the docking station 5 or input/output device 13, etc.). In other embodiments, the collected and evaluated data as well as data identifying when an output was provided to the sleeping patient and what that type of output was provided to the sleeping patient can be stored in the memory of the wearable device 3 and subsequently transmitted to the docking station 5, input/output device 13, and/or central server 7 for storage and further analysis, use, and evaluation as discussed herein.

The wearable device 3 can be configured to activate one or more of its sensors 4 of its sensor array at a pre-selected time interval for periodic measurement collection that can be used to trigger an automatic response to try and reduce a user's detected anxiety that may be detected as exceeding a pre-selected anxiety threshold level. The process shown in FIG. 16 can be utilized to detect a user's anxiety and subsequently trigger actuation of output to facilitate a reduction in the user's detected anxiety. For at least some of the sensors 4, the wearable device 3 can be configured so that the sensors are continuously collecting data from the user. In other embodiments, all the sensors 4 can be activated at different pre-selected time intervals for the collection of measurement data of the user while the user is wearing the wearable device 3.

The wearable device 3 can be configured so that different sensor data is received and evaluated for a pre-selected evaluation time period (e.g. a time period of 1 minute, 2-3 minutes, 5 minutes, etc.). The sensor data can include heart rate sensor data, sweat sensor data, and accelerometer sensor data. The wearable device 3 can be configured to evaluate the sensor data to determine whether the patient has measurement data that exceeds a pre-selected anxiety threshold. Such a threshold can include a collection of parameters. For example, if the patient heart rate is over a user baseline (e.g. 75 beats per minute (bpm) or 70 bpm, etc.), and the patient is also detected as sweating and having accelerometer data indicating the patient has been having one or more tremors that meet or exceed a pre-selected anxiety threshold rate, the wearable device 3 can be configured to detect an anxiety condition and, as a result, further evaluate the patient objective measurement data for a pre-selected period of time to determine if the patient is experiencing an increase in body movement, sweat, and/or relatively high heart rate (HR) that meets or exceed a pre-selected anxiety threshold rate. For example, in some embodiments, such a determination can also be made in response to detecting that the user is experiencing body motion that meets or exceed a pre-selected anxiety threshold rate. The input/output device 13, central server 7, or docking station 5 can be configured to evaluate the user data, or patient data, as well after that data is received from the wearable device 3 for use in the evaluation of the patient's monitored health related data for use in determining different anxiety related health parameters.

If the frequency of tremors is increasing and/or the body movement is increasing, and the heart rate (HR), and sweat data of the patient exceed a pre-selected anxiety threshold, the wearable device 3 can be configured to communicate with at least one input/output device 13 and/or the docking station 5 or central server 7 to cause at least one output to be emitted while the patient is sleeping and/or actuate a speaker or other mechanism of the wearable device to emit the output. For example, the wearable device can be configured to vibrate via a vibration mechanism to provide a sensory and/or audible output that may help reduce patient anxiety. The vibration mechanism can be configured as a vibration motor, a shaftless vibration motor, a linear resonant actuator, a vibration motor that produces an oscillating force across a single axis, or an eccentric rotating mass (ERM) motor that is connected to the wearable device housing. The vibration mechanism can be configured so that when the vibration mechanism is actuated, the housing of the wearable device or the entire wearable device 3 can vibrate. In addition, the wearable device 3, input/output device 13 or docking station 5 can output an audible output via a speaker by playing a particular type of music or sound that the patient can hear to try and improve the anxiety of the patient.

In the event the frequency of the patient movement, sweat, and heart rate is not increasing and these values are not above a pre-selected anxiety threshold, the wearable device 3 can be configured to stop collecting data of the patient for a pre-selected period of time to preserve the battery life of the wearable device 3. After that pre-selected delay period of time is reached, the wearable device 3 can re-activate the sensors 4 for collection and evaluation of the data to evaluate whether the patient is experiencing a level of anxiety that meets or exceeds the pre-selected anxiety threshold.

The wearable device 3 can also be configured so that the patient data that is collected while the patient is wearing the device and that data can be periodically transmitted to the docking station 5, input/output device 13, and/or central server 7 for storage and further analysis or evaluation. Such periodic transmission can occur while the patient is wearing the device and the sensors are activated to collect the patient data after each such activation sequence and can also include data identifying the time that an output was provided to the patient and what type of output was provided to the patient (e.g. just vibration, vibration and music via a speaker of the wearable device 3 or a speaker of the docking station 5 or input/output device 13, etc.). In other embodiments, the collected and evaluated data as well as data identifying when an output was provided to the patient and what that type of output was provided to the patient can be stored in the memory of the wearable device 3 and subsequently transmitted to the docking station 5, input/output device 13, and/or central server 7 for storage and further analysis, use, and evaluation as discussed herein.

The wearable device 3 can be configured to detect and count tremors a user may experience. The tremor related data can be communicated to the input/output device 13 and/or docking station 5. The central server 7, input/output device 13, docking station 5, and/or wearable device can also be configured to utilize this data to generate a tremor score that may be used in the evaluation of the user's health as well.

The tremor that is detectable by the wearable device can include a tremor associated with a neurological disorders, including, for example, multiple sclerosis, stroke, and traumatic brain injury, and neurodegenerative diseases that affect parts of the brain, including, for example, Parkinson's disease, attention deficit hyperactivity disorder (ADHD), attention deficit disorder (ADD), dementia, or Alzheimer's disease. The user tremors that are detectable for evaluation can also include tremors which result from the use of certain medicines (for example, asthma medication, amphetamines, caffeine, corticosteroids, and drugs used for certain psychiatric and neurological disorders), alcohol abuse or alcohol withdrawal, overactive thyroid, liver failure, kidney failure, anxiety or panic. A number of different forms of tremor exist and may be detected, including, for example, essential tremor (also known as benign essential tremor or familial tremor), dystonic tremor, cerebellar tremor (e.g., tremors caused by damage to the cerebellum and its pathways to other brain regions resulting from a stroke or tumor, or caused by disease (such as multiple sclerosis or an inherited degenerative disorder, e.g., ataxia and Fragile X syndrome) or resulting from chronic damage to the cerebellum due to alcoholism), psychogenic tremor (also called functional, and includes tremors resulting from stress or a psychiatric disorder such as depression or post-traumatic stress disorder (PTSD)), enhanced physiological tremor (e.g., tremors resulting from certain drugs, alcohol withdrawal, or medical conditions including an overactive thyroid and hypoglycemia, Parkinsonian tremor, and orthostatic tremor.

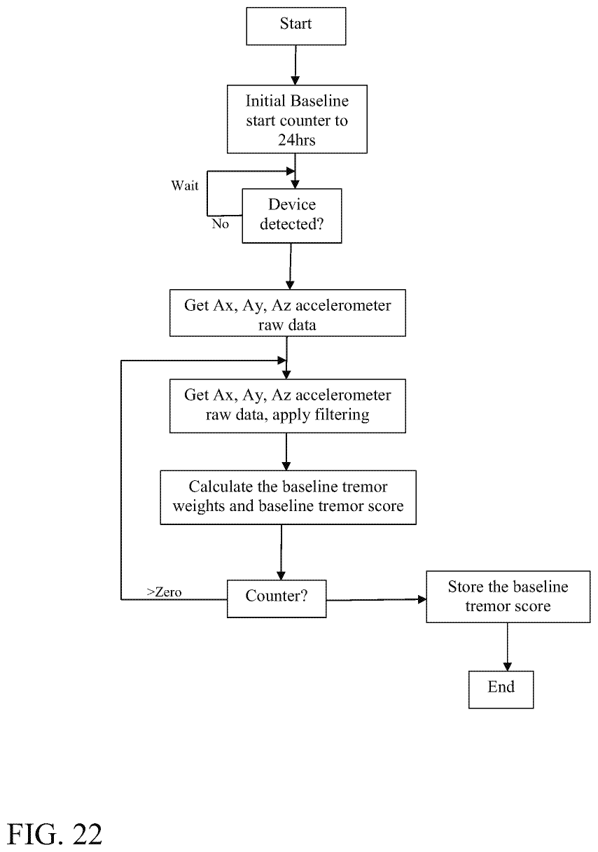

Referring to FIG. 22, user tremors can be detected for evaluation after a baseline is formed. To form a baseline for tremor evaluation, a tremor baseline time period can be set for recording sensor data for the tremor baseline time period. This time period can be 24 hours, multiple days, or a week, for example. After this time period has elapsed, sensor data collected during the tremor baseline time period can be evaluated. This data can include accelerometer data that includes detected motion data along multiple axes. For instance, the accelerometer data can include accelerometer x direction data Ax, accelerometer y direction data Ay, and accelerometer z direction data Az collected for each time segment of a particular measurement time. The x direction can be horizontal, the y-direction can be vertical (e.g. a direction along an axis that extends vertically upward and downward), and the z direction can be a direction that is perpendicular to vertical and perpendicular to the horizontal x direction. For example, the x direction can be a direction along a horizontally extending axis that extend horizontally in right and left side directions and the z direction can be a direction along a horizontal axis that extends in front and rear directions. This accelerometer data can be filtered and subsequently used to calculate baseline tremor weights and a baseline tremor time score. The filtering that is performed can be to filter out the accelerometer data that does not meet or exceed a pre-determined tremor threshold level of tremor motion. The baseline accelerometer data that is collected within the tremor baseline time period can be evaluated to detect:

1. The number of tremors that happened within the baseline time period, which can be considered a Tremor Count Tc;

2. The amount of time elapsed during occurrence and non-occurrence of a detected tremor, which can be considered a Tremor Duration T_{D};

3. An amplitude of the tremor, which can be a measured change in the detected tremor within a single period of the tremor, which can be considered Tremor Amplitude T_{A}; and

4. A number of tremor occurrences within a particular unit of time, which can be considered the tremor frequency T_{F}.

Based on the baseline tremor data, varying weights can also be determined. The weights can include a first weight w1, a second weight w2, a third weight w3, and a fourth weight w4. The baseline tremor data and weights can be utilized to calculate a baseline tremor score, Ts. The tremor score can be calculated according to the following formula in some embodiments:

T_{S}=(w1*T_{F}+w2*T_{A}+w3*T_{D}+w4*T_{C})*100 (Tremor Score Equation 1).

In other embodiments, a different scoring calculation can be utilized. For instance, the tremor score may not utilize the value 100 in some embodiments to generate the score and may instead be: calculated by use of the following tremor score calculation:

T_{S}=w1*T_{F}+w2*T_{A}+w3*T_{D}+w4*Tc (Tremor Score Equation 2).

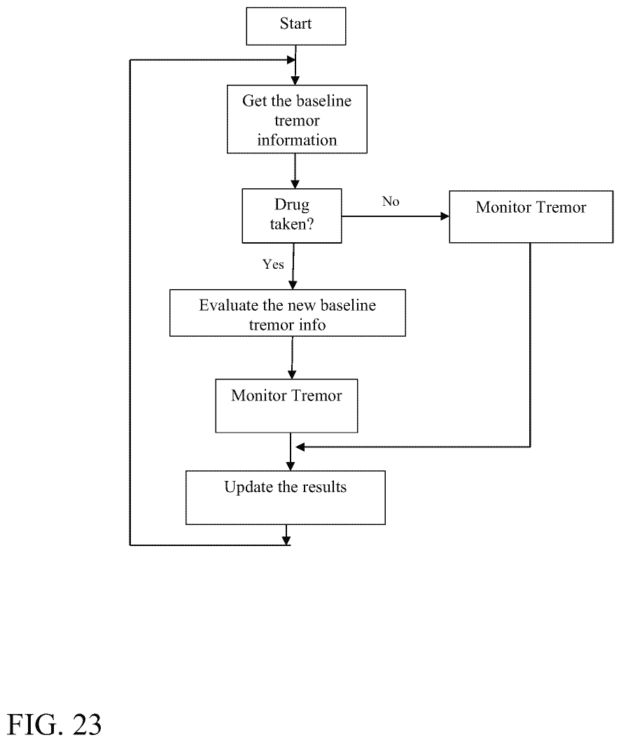

After a baseline tremor evaluation is made and a baseline tremor score is determined, a user may utilize a drug or other type of medical intervention to try and improve his or her condition. Such an improvement (or lack thereof) can be evaluated by comparing the tremors a user may experience while using the medical intervention, which can also be referred to herein as a behavioral change (e.g. drug, meditation, yoga, or other intervention), with the baseline tremor data and baseline tremor score that was obtained prior to that intervention. FIG. 23 illustrates an exemplary process by which such comparison data can be obtained and utilized.

For example, after the baseline tremor information is obtained as discussed herein, the wearable device 3, input/output device 13, or central server 7 can receive data indicating the user is undergoing an intervention, such as taking a newly prescribed medication, changing the user's diet, or changing a user activity (e.g. engaging in meditation or yoga at an increased frequency etc.). In response to that first input concerning the intervention, the wearable device 3 can be used to evaluate a new baseline tremor score for the user after the user has engaged in the intervention activity. The new baseline tremor score can be obtained using the same process discussed above with reference to FIG. 22. For example, the user's tremors can be detected for evaluation after the user engages in the intervention to obtain a new baseline score. The new tremor baseline can be determined after an intervention timer period has passed after the intervention was initiated. For instance, the determination of the new baseline can be initiated one day after a new drug is prescribed and taken or after 30 days of the new drug being taken if it may take 30 days before the drug is likely to be effective. Other new baseline time periods can also be utilized for different interventions to meet the particular time related properties of the particular intervention being utilized for evaluation and comparison of that intervention and its effect on the user.

The new tremor baseline can be determined by collecting sensor data for tremor detection and evaluation within a new tremor baseline time period. This time period can be set for recording sensor data for the new tremor baseline time period. This time period can be 24 hours, multiple days, or a week, for example. After this time period has elapsed, sensor data collected during the tremor baseline time period can be evaluated. This data can include accelerometer data that includes detected motion data along multiple axes. For instance, the accelerometer data can include accelerometer x direction data Ax, accelerometer y direction data Ay, and accelerometer z direction data Az collected for each time segment of a particular measurement time. The x direction can be horizontal, the y-direction can be vertical (e.g. a direction along an axis that extends vertically upward and downward), and the z direction can be a direction that is perpendicular to vertical and perpendicular to the horizontal x direction. For example, the x direction can be a direction along a horizontally extending axis that extend horizontally in right and left side directions and the z direction can be a direction along a horizontal axis that extends in front and rear directions.

The accelerometer data can be filtered and subsequently used to calculate new baseline tremor weights and a new baseline time score. The filtering that is performed can be to filter out the accelerometer data that does not meet or exceed a pre-determined tremor threshold level of tremor motion.

Based on the new baseline tremor data, varying weights can also be determined. The weights can include a first weight w1, a second weight w2, a third weight w3, and a fourth weight w4. The new baseline tremor data and weights can be utilized to calculate the new baseline tremor score, Ts. The new tremor score can be calculated according to the same aforementioned formula(s) used to form the baseline tremor score (e.g. Tremor Score Equation 1 or Tremor Score Equation 2, etc.). The new tremor score can then be compared with the old baseline score (e.g. a second tremor baseline score can be compared to a first tremor baseline score) to determine if the intervention has provided an improvement or had no meaningful effect. The evaluation can include, for example, comparing the difference between first and second tremor baseline scores with a pre-selected efficacy value that is defined to help evaluate whether a change of statistical significance has occurred in the user's tremor baselines that were determined from the comparison. If no improvement or meaningful effect is found to have occurred, a change to the intervention can be made (e.g. increase dosage of new drug from a first dosage to a second dosage, adjust frequency of taking the drug from a first frequency to a second frequency (e.g. from once a day to once every 12 hours or once a day to once every six hours, etc.), change to the medication taken by the patient, further adjust user activity or diet, etc.). After the change in dosage of the drug from the first dosage to the second dosage, drug taking frequency (e.g. a change from a first drug frequency to a second drug frequency), or other change is made, the second tremor baseline score can be compared to a third tremor baseline score that is obtained using the similar process used to generate the second tremor baseline score after this change has begun (e.g. using the sensors to obtain additional sensor data, etc. for the second change in dosage, frequency, and/or other behavior and processing that data as discussed above). The third tremor baseline score can then be compared to the first tremor baseline score and/or the second tremor baseline score to evaluate whether the additional change has a meaningful effect. If no improvement is provided, yet another change can again be made to either drug dosage (e.g. a third dosage can be utilized as another change), frequency (e.g. a third drug frequency can be utilized as another changer), or another parameter. This process of drug dosage or frequency adjustment (or other activity adjustment) can be iteratively performed multiple times repeatedly until a meaningful improvement has been obtained (e.g. there may be changes and sensor data obtained from a fourth drug dosage, fourth drug frequency, and fourth baseline tremor score, . . . etc.). The time taken between performing the tremor score baseline calculations can be considered a pre-selected improvement time period (e.g. a time period after the change would be expected to result in a meaningful change in the user's health). In some embodiments, the new baseline score may not be calculated until the user provides a second input related to the change in dose, frequency of drug taking, or other change to rigger the calculation or determination of the new baseline score.

The evaluation of tremors and calculation of tremor score can be performed by the input/output device 13, central server 7, and/or wearable device 3 depending on the configuration of a particular apparatus 1. In some embodiments, the central server 7 can perform the calculation based on the wearable device's sensor data it receives via its connection with an input/output device 13 or docking station 5. The tremor score information can then be communicated to the user via the input/output device 13 or docking station 5 via a GUI of that device that can be output by a display 13a and the device's communicative connection with the central server 7. In other embodiments, the input/output device 13 (e.g. smartphone, laptop, or tablet, etc.) can perform the evaluation by running an application stored thereon that may be hosted or supported by the central server 7 by using the sensor data received from the wearable device 3.

The user's sleep or other health condition can also be monitored in conjunction with utilization of a combination of subjective and objective data. For example, a mind, body, and diet (MBD) score can be calculated to provide a MBD score that helps evaluate a patient's health or the quality of sleep the patient may be getting. The MBD score can be calculated based on MBD weights such as first and second MBD weights w1_{MBD} and w2_{MBD}. The first MBD weight w1_{MBD} can be used to provide a weight for the subjective mind and body score MB input a user may provide and the second MBD weight w2MBD can be used to weigh a diet score D for the user based on dietary information obtained from the user. The MBD score can be calculated according to:

MBD score=MB*w1_{MBD}+D*w2_{MBD} (MBD score equation 1)

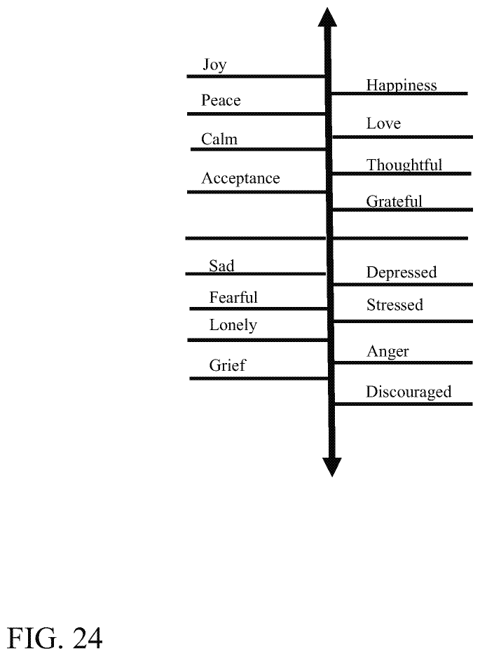

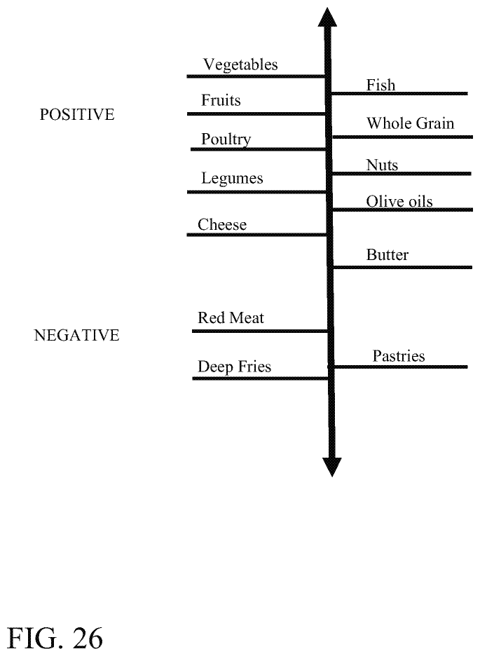

FIGS. 24 and 26 illustrate scales that can be utilized for providing the MB score and the D score that can be generated based on user input received about the user's diet and mind and body input. For example, an input/output device 13 can generate a GUI that solicits the user to indicate the user's mind and body score (MB) by selecting a term that may best describe how the user is feeling at that time. Entering input indicated joy or happiness can generate a higher score than providing input corresponding to grief, anger, or discouraged, for example. The selection of a particular input value can be assigned a pre-selected MB value corresponding to that input. The MB score can also be considered an Emotion score, or EQ score. For such embodiments, the MBD score can be considered an EQ score and diet score or an ED score.

For a diet score (D), the input/output device 13 can generate a GUI that solicits the user to indicate the user's diet score (D) by selecting dietary choices the user has made that may best describe the user's diet. The input that is provided related to a diet can indicate the type of food eaten, such as fish F, vegetables V, fruits FR, whole grains WG, poultry P, nuts N, legumes L, olive oil 00, cheese C, butter B, red meat RM, deep fried foods DFF or pastries PS, as well as the amount of these foods eaten in a particular meal or during a particular day. Other embodiments may utilize different dietary entry options that are more or less than this exemplary set of entry options. This dietary data can be used for the generation of a diet score D. The diet score valuations for different dietary input can be configured so that a higher score is generated for a person who eats more healthy food as compared to a selection of dietary items that may be associated with junk food or other less nutritious food (e.g. candy, corn chips, pastries, etc.) The selection of a particular input value for diet received from the user can be assigned a pre-selected value corresponding to that dietary input, frequency or amount of that food eaten X, and a diet score weight W_{nD}. For example, an overall diet score D can be calculated based on the dietary information obtained from the user according to the following calculation:

Diet Score D=ΣW_{nD}*n*X, where n is the dietary option. (Diet Score Equation 1).

For example, this calculation can be utilized in the above referenced example as:

Diet Score D=w_{1D}*V*X+w_{2D}*F*X+w_{3D}*FR*X+w_{4D}*WG*X+w_{5D}*P*X+w_{6D}*N*X+w_{7D}*L*X+w_{4SD}*C*X+w_{9D}*B*X+w_{10D}*RM*X+w_{11D}*DF*X+w_{12D}*PS*X+w_{13D}*OO*X. (Diet Score Equation 2).

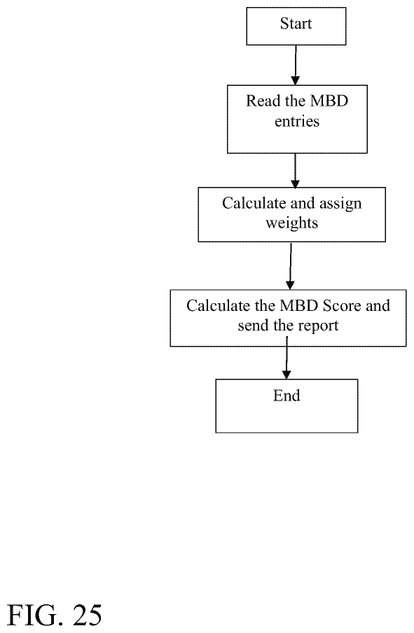

As may be appreciated from FIG. 25, after the user entered MB and D input is obtained and MB and D scores are determined, the weights to those scores can be generated and applied to form the MBD score. The score and a report related to the user input that was entered and saved can be transmitted to the user via the input/output device and can also be sent to another device (e.g. wearable device data management device 7a and also storable in a patient data management device 7b) for storage and subsequent use.

The MBD score can be calculated by the input/output device 13, the docking station 5, or the central server 7 based on data received from the wearable device 3 or based on the input obtained from the user via the GUI of the input/output device 13 or docking station 5, which may have been generated in conjunction with an API formed via a connection with the central server 7. In yet other embodiments, the wearable device 3 may facilitate receipt of such input and output of such score data via a GUI it generates via its display 13a (if present).

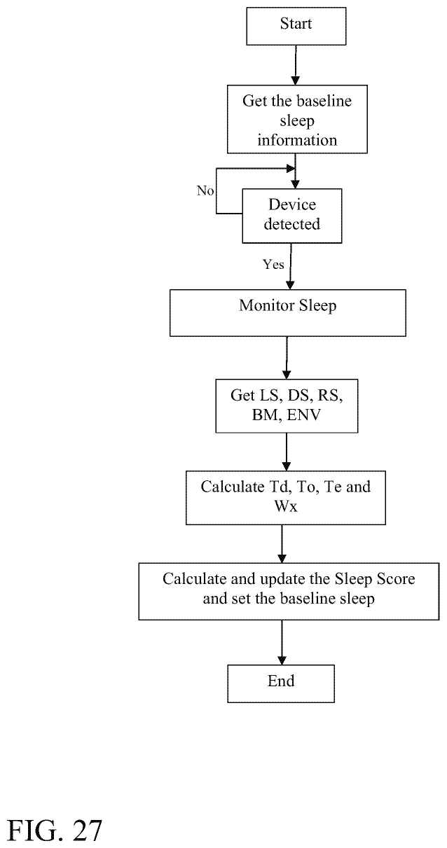

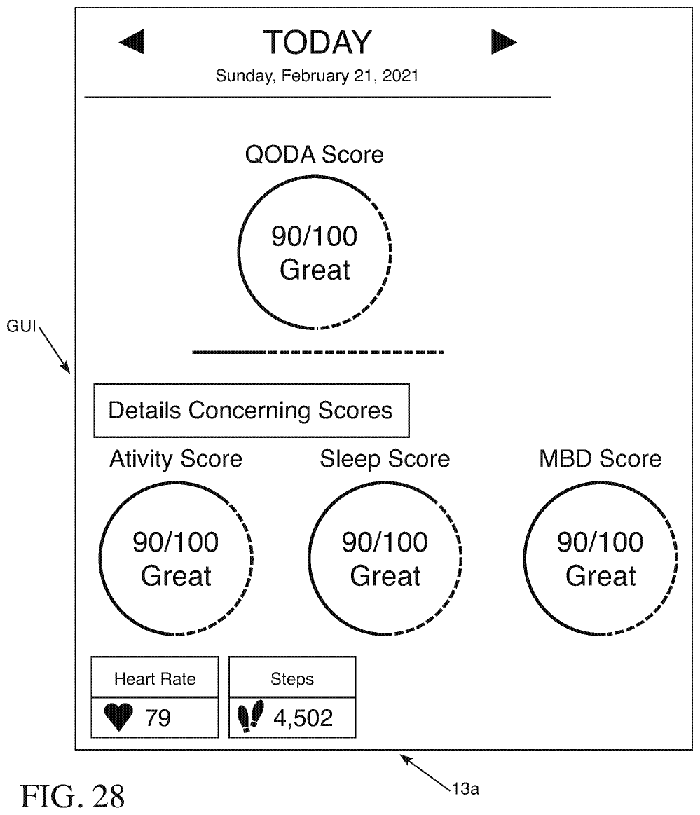

Referring to FIG. 27, the sensor data as well as user input can be utilized to determine a sleep score. The sleep score and other scores can also be combined for use in determining a quality of daytime activity (QODA) score as well. These calculated scores can be displayed to a user via the GUI of a display 13a of the wearable device 3, input/output device 13, or docking station 5 as shown in FIG. 28, for example.

As can be appreciated from FIG. 27, an exemplary sleep score calculation process can be performed utilizing sensor data collected via the wearable device 3. The wearable device 3, input/output device 13, docking station 5, or central server 7 can be configured to utilize the sensor data and perform this calculation. as well as provide data for the generation of a graph, chart, text, or other display of information via a GUI for providing information to a user about the sleep score for display via a display 13a.

User baseline sleep information can be retrieved from stored data. This sleep information can include past user sleep concerning REM sleep (RS), body movement that occurs during sleep (BM), environmental effect (ENV) related to detected noise or light that was in the environment around the user while the user slept, sleep information concerning light sleep (LS), sleep data related to deep sleep (DS), and total time sleep (Td). Additionally, sensor data related to a detected sleep onset (To) and a sleep efficiency value (Te) can be utilized. The sensor data can be evaluated to determine when the user slept, and how long the user slept to obtain the Td and To values. The sensor data can also be used to ascertain the DS, LS, ENV, BMₛₗₑₑₚ, and RS values. Weighting values for each parameter Wₛₗₑₑₚ, can also be determined. These parameters may be pre-defined or can be within pre-defined ranges based on the values of the parameters to which the weight is to be applied. After these values are determined, the sleep score can be calculated. This sleep score can also be used to set a baseline sleep score for comparison with subsequently calculated sleep scores for different time periods. The sleep score can be calculated in accordance with the following sleep score calculation:

Sleep Score=f(Wnₛₗₑₑₚ,LS,DS,RS,MB,ENV,Td,To,Te} (Sleep Score Equation 1).

For example, the sleep score can be calculated in accordance with the below equation:

Sleep Score=W_{LS}*LS+W_{DS}*DS+W_{RS}*RS+W_{BMsleep}*BMₛₗₑₑₚ,+W_{ENV}*ENV+W_{Td}*Td+W_{To}*To+W_{Te}*Te (Sleep Score Equation 2).

### In Sleep Score Equation 2:

W_{LS} is the weight for LS;
W_{DS} is the weight for DS;
W_{RS} is the weight for RS;
W_{BMsleep} is the weight for BMₛₗₑₑₚ,
W_{ENV} is the weight for ENV;
W_{Td} is the weight for Td;
W_{To} is the weight for To; and
W_{Te} is the weight for Te.

In some embodiments, the weights can be determined such that the weight for light sleep W_{LS} is in a range of 0.02-0.10 (2%-10%) or 0.05-0.07 (5%-7%), the weight for deep sleep, W_{DS} is in a range of 0.45-0.75 (45%-75%) or 0.50-0.60 (50%-60%), and the weight for REM sleep, W_{REM} is in a range of 0.23-0.53 (23-53%) or 0.20-0.30 (20%-30%). The W_{DS}*DS term of the sleep score calculation can be broken into different components for the first and second stages of deep sleep. For such embodiments, the weight for deep sleep of stage 1 (W_{DS1}) can be in the range of 0.40-0.60 (40%-60%) and the weight for deep sleep in stage two (W_{DS2}) can be in the range of 0.10-0.30 (10%-30%). An example of such a sleep score calculation involving deep sleep stages is provided below:

Sleep Score=W_{LS}*LS+W_{DS1}*DS1+W_{DS2}*DS2+W_{RS}*RS+W_{BMsleep}*BMₛₗₑₑₚ,+W_{ENV}*ENV+W_{Td}*Td+W_{To}*To+W_{Te}*Te (Sleep Score Equation 3).

### In Sleep Score Equation 3:

W_{LS} is the weight for LS;
W_{DS1} is the weight for stage 1 DS;
W_{DS2} is the weight for stage 2 DS;
W_{RS} is the weight for RS;
W_{BMsleep} is the weight for BMₛₗₑₑₚ,
W_{ENV} is the weight for ENV;
W_{Td} is the weight for Td;
W_{To} is the weight for To; and
W_{Te} is the weight for Te.

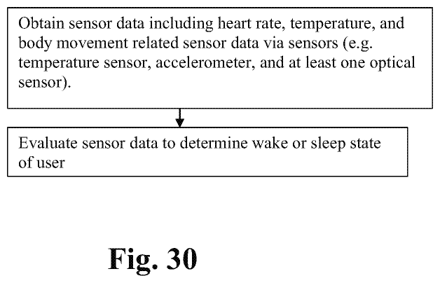

Referring to FIGS. 30-34, the sensor data can be utilized to determine the sleep parameters for the sleep score calculations as well as monitor the different sleep states of a user as well as a wakefulness state of the user for evaluation of the user's sleep over an empirical time period to help provide an evaluation of the user's sleep as well as provide an efficacy evaluation for determining whether sleep has improved or not based on a particular treatment the user may be engaged in (e.g. taking a medication, increasing the dose of a medication, changing the frequency of the dose of a medication being taken by the user, etc.).

For example, the sensor data can be obtained and subsequently evaluated to determine whether the user is sleeping or awake. The time frame for which a user is determined to be awake can be tracked via time information obtained via the clock or timer of the wearable device. The time frame for which a user is determined to be sleeping as well as the time a user spent in different sleep states can also be tracked via time information obtained via the clock or timer of the wearable device for the obtained sensor data.

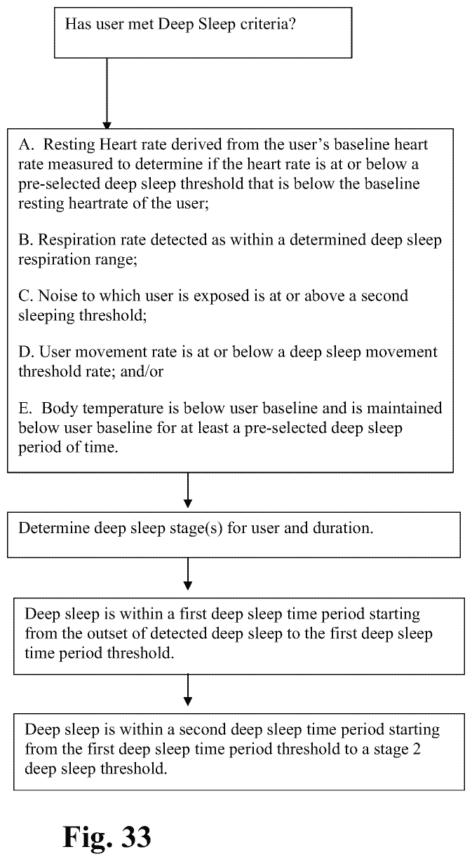

In some embodiments, the user's sleep states can include multiple states that include light sleep, deep sleep, and REM sleep. The user's sleep state can also include a first stage of deep sleep and a second stage of deep sleep as can be appreciated from FIG. 33, for example. The user may be determined to have shifted between different sleep states based on the user sensor data provided by the sensors of the wearable device 3. The sensor data can include, for example, temperature data via a temperature sensor, heart rate data via at least one optical sensor, and accelerometer data that can indicate an extent of motion by a user or a rate of motion by a user. Other sensor data can also be utilized.

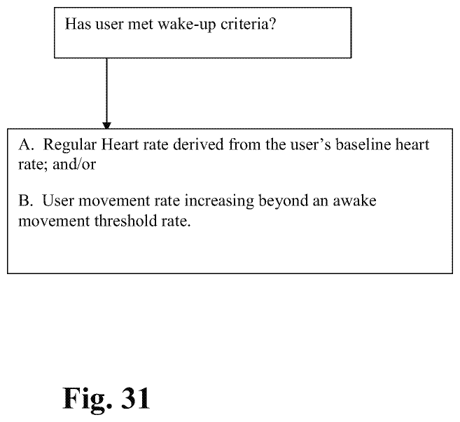

The user can be determined to be awake in response to the sensor data indicating that the user meets a pre-selected set of wake-up criteria or awake criteria. FIG. 31 illustrates one exemplary process for this type of determination. The awake criteria can include the use's regular heart rate that can be derived from the user's baseline heart rate. For example, a heart rate of 72 beats per minute (bpm) can be considered a baseline heart rate based on the user's empirical heart rate data obtained by the sensors of the wearable device. The user's baseline heart rate can be derived based on the user's detected heart rate over a period of weeks or months. This baseline hear rate, which can also be referred to a “regular heart rate,” can be updated periodically to account for changes that may occur in the user's hear rate over such a time period.

In addition to the heart rate being at or above a baseline heart rate of the user, user body movement can be evaluated based on the accelerometer data and time of that data. The user can be considered to have awaken from sleep when the user is determined to have a heart rate at his or her regular heart rate in addition to the user's detected body movement being above a sleep movement threshold and/or be continuously increasing over a pre-selected waking up time period (e.g. the user is determined to have exceeded a pre-selected step count for the pre-selected waking up time period). The sleep movement threshold can be a movement rate within a pre-selected sleep movement time period or an absolute amount of motion that may be detected within a particular time period.

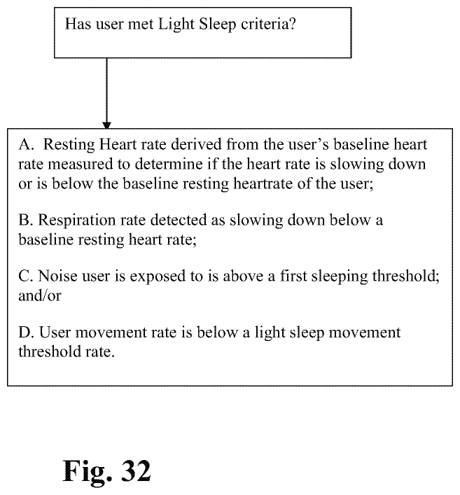

The user can be determined to be in a light sleep state (e.g. LS) based on a determination made using light sleep criteria. FIG. 32 illustrates one example of such a determination process. The light sleep criteria that can be evaluated for determination of an extent to which a user experienced light sleep during his or her sleeping time can be based on the sensor data meeting the light sleep criteria for a particular time period. The light sleep criteria can include the user's detected resting heart rate slowing below the resting heart rate baseline of the user (e.g. if the user baseline resting heart rate is 72 beats per minute, bpm, then a detection of the user heart rate slowing to under 72 beats per minute (bpm) can meet this aspect of the light sleep criteria for the user). In some embodiments, the light sleep heart rate can be determined if the heart rate is below the user's baseline resting heart rate and above the user's deep sleep threshold heart rate value.

Additionally, the sensor data can show that the user's respiration rate slowed below a user's resting respiration rate for the user to be determined to meet the light sleep criteria. The audible noise near the user detected by the microphone can also be utilized to help detect light sleep of a user. If the user is determined to have a suitably low heart rate below the resting hear rate as well as a slowed respiration rate below a baseline respiration rate of the user and the noise is also detected as being above a light sleep noise threshold (e.g. a noise threshold of 30 dB, 35 dB, 25 dB, a noise of at least 30 dB, etc.), this can also be an additional factor used to determine the user is in a light sleep state. User body movement can also be used to determine the user's light sleep state. For instance, when the user's movement is at or below a light sleep movement threshold in addition to the user's heart rate being below the baseline resting heart rate and the respiration rate of the user being below the baseline resting respiration rate, the user can be determined to be in a light sleep state for this time period. The user's light sleep state can also be further clarified by accounting for other sleep state determinations. If the user is determined to be asleep and is not within the REM or deep sleep state criteria, the user can be considered to be in a light sleep state.

For most users, it would be expected for the user to have at least 50% of his or her sleeping time be at the light sleep state classification. However, it is possible the light sleep classification can be set so that this light sleep state may be lower than this expected range in some embodiments.

The user's deep sleep can be determined form the sensor data as well. The deep sleep criteria can be evaluated and considered met based on evaluation of the sensor data of the wearable device 3. An exemplary process for deep sleep evaluation and tracking can be appreciated from FIG. 33. Deep sleep of the user can be determined if the deep sleep criteria is met via evaluation of the sensor data of the wearable device 3. This criteria can include the user's heart rate being at a deep sleep threshold or below the deep sleep threshold. The deep sleep threshold can be determined to be 10-25% below the user's baseline regular resting heart rate. In some embodiments the deep sleep threshold can be 10-15% below the user's baseline resting heart rate, or at least 10% below the user's baseline resting heart rate. The selected deep sleep threshold can also be selected so that it is above a pre-selected light sleep heart rate range.

In addition to having a required heart rate (HR), the user can also have to have a respiration rate that is within a pre-selected deep sleep range that can be below a light sleep respiration rate range, the user's body temperature is within a pre-selected deep sleep temperature range, and the user's detected body motion can be within a pre-selected deep sleep body motion range. Noise near the user can also be utilized to help evaluate the sleep state of the user. For instance, if noise detected by the microphone is at or above a pre-selected deep sleep noise level, which may be higher than the pre-selected light sleep noise level, and the user's motion, respiration rate, and heart rate all meet their deep sleep targets, the user can be determined to be in a deep sleep or can have a higher estimated certainty of being in a deep sleep state.

For a deep sleep determination, the user's body motion deep sleep range can be negligible to no detected body movement within a particular sampling time period. For example, if a user is detected as having less than a deep sleep number of movements within a sampling time period, the user's body movement can be considered negligible and this can meet the deep sleep criteria for body motion.

For user body temperature, the deep sleep temperature range can be found to be met when the user temperature is determined to be below the user's baseline temperature that is determined to be the user's body temperature at rest when awake based on empirical recorded temperature data of the user and is found to be within the deep sleep temperature range for at least a pre-selected deep sleep temperature time period (e.g. at least 5 minutes, at least 10 minutes, etc.).

The deep sleep of the user can also be evaluated to categorize the deep sleep as falling within a first stage or a second stage of deep sleep. Deep sleep in a first stage of deep sleep can be any time from when the onset of a deep sleep timer period is detected to a first stage ending time threshold, which can be a particular pre-defined time period for stage one deep sleep. This time period can be 45 minutes, 60 minutes, or another time between 45-90 minutes, for example. A second stage of deep sleep can be determined to exist for a time period in which the user is found to have been in deep sleep continuously since the determined onset of deep sleep that occurs after the first stage ending time threshold has been met (e.g. the time in which the user stayed in a deep sleep state after the first stage ending time threshold was met, and before the user was determined to have left a deep sleep state).

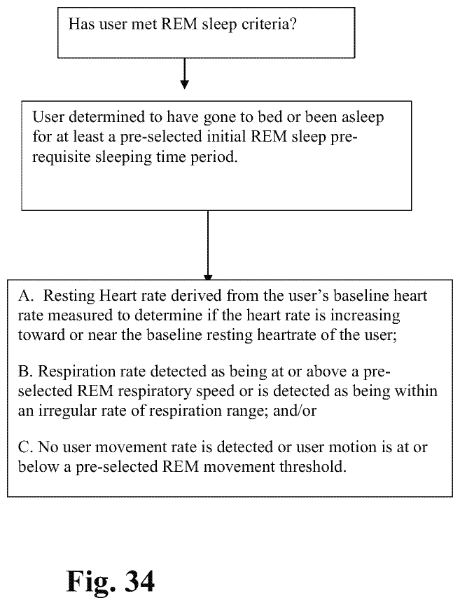

The user's rapid eye movement (REM) sleep can additionally be determined form the sensor data. The REM sleep criteria can be evaluated and considered met based on evaluation of the sensor data of the wearable device 3. An exemplary process for REM sleep evaluation and tracking can be appreciated from FIG. 34. REM sleep of the user can be determined if the REM sleep criteria is met via evaluation of the sensor data of the wearable device 3.

The REM sleep criteria can include the user's hear rate being determined to be increasing toward the user's baseline resting heart rate. The REM sleep criteria can also include body movement data from the accelerometer. If no or negligible motion is detected, the user can be found to meet the REM sleep state body motion criteria. The user's respiration rate being within a REM respiration level can also be utilized. To meet the REM respiration rate, the user respiration rate can be irregular and/or higher than the light sleeping or deep sleeping respiration rate. The REM criteria can also include a requirement that the user be determined to be in a sleeping state continuously for at least 90 minutes. Any sleep data that may otherwise meet the REM sleep state criteria but is obtained less than 90 minutes of an onset of a user having been determined to be asleep may not be considered a REM sleep state, but may instead be considered a light sleep state or a deep sleep state.

The QODA score can be a summation of all these other scores or a subsect of such scores. For example, the QODA can be the sum of the sleep score and the MBD score or the sleep score, the MBD score and an activity score. The QODA can also be a modification of such sums to place these values in a percentage form that can range from 0% to 100%. For instance, the QODA score can be calculated by use of the formula:

QODA=Sleep Score+MBD Score (QODA score equation 1)

As another example, the QODA score can be calculated by use of the formula:

QODA=Sleep Score+MBD Score+Activity Score (QODA score equation 2)

As yet another example, the QODA score can be calculated by use of the formula:

QODA=Sleep Score*W_{QODASS}+MBD Score*W_{QODAMBD} (QODA score equation 3) or

QODA=Sleep Score*W_{QODASS}+MBD Score*W_{QODAMBD}+Activity Score*W_{QODAAct} (QODA score equation 4)

where:
W_{QODASS} is a QODA weight for the Sleep Score;
W_{QODAMBD} is a weight for the MBD Score; and
W_{QODAAct} is a weight for the Activity Score.
The weights used in QODA score equations 3 and 4 can be pre-selected or pre-defined based on a set parameter or a range of set parameters that are selectable based on user data obtained via the wearable device 3, subjective data obtained from the user, and/or other information.

The activity score can be calculated based on accelerometer data as well as other sensor data that can be used to determine the level of activity of the user. The activity score can also be updated to account for user input that may be provided to indicate other activities the user may have engaged in such as stationary bike riding, weight lifting activities, yoga, playing a sport, or other activities.

For example, in some embodiments, the activity score can be determined based on accelerometer data indicating movement of the user. The movement data obtained from the accelerometer as well as other sensors can be used to determine the type of activity the user is engaged in (e.g. walking, running, etc.) and can also determine a number of steps the user has taken in a particular time period (e.g. a day, a week, a month, a year, etc.). For example, the accelerometer data can be evaluated to determine the speed of each stride a user has taken and the length of the stride. When the detected stride length and speed of each stride is at or above a running threshold, the user can be determined to be running. When the detected stride length and speed of each stride is below the running threshold and at or above a walking threshold, the user can be determined to be walking.

The user can also provide input to facilitate such activity related determinations, such as input indicating an activity goal (e.g. taking 10,000 steps a day, etc.) as well as how tall the user is and/or what the user's stride length is. The user can also enter activity data input such as a duration of time spent performing a particular activity or a distance the user may have ran, walked, rode a bicycle, swam, or engaged in another type of activity. The wearable device 4, central server 7, docking station 5, and/or input/output device 13 can be configured to utilize such data in conjunction with the sensor data to determine the activity type, extent of activity (e.g. number of steps taken, etc.) and how that activity compares to the user's goal to determine an activity score. For example, if a user has a goal of taking 10,000 steps a day, and the user in a particular day took 9,000 steps, the user's activity score can be determined to be 90% or 0.90.

In some embodiments, the activity score can be determined using the combination of heart rate and accelerometer data to determine the intensity of the activity or activities the user has engaged in. For example, the heart rate sensors can detect the increasing heart rate while performing a detected activity, and the heart rate data can be combined with the accelerometer data to detect the type of activity and its intensity to determine the activity score.

As with the exemplary sleep score calculation process as well as other exemplary score processes discussed herein, the activity score process can be performed utilizing sensor data collected via the wearable device 3. The wearable device 3, input/output device 13, docking station 5, or central server 7 can be configured to utilize the sensor data and perform this calculation. as well as provide data for the generation of a graph, chart, text, or other display of information via a GUI for providing information to a user about the sleep score for display via a display 13a.

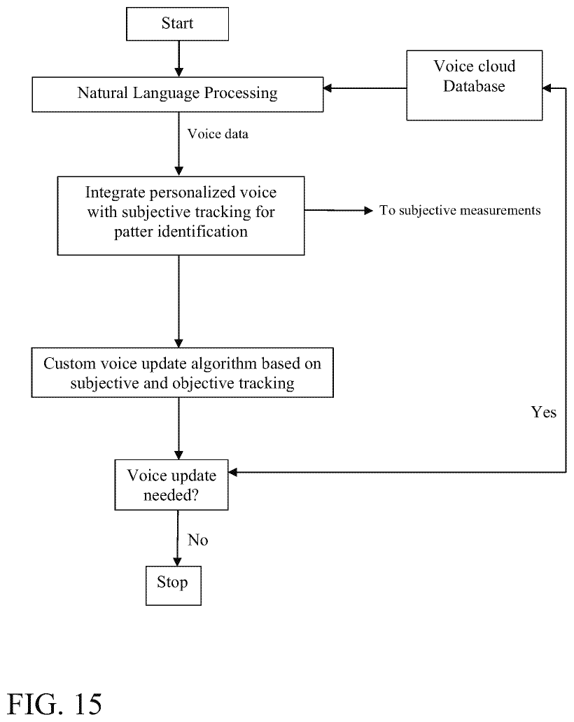

The wearable device 3 and the input/output device 13 (e.g. smart speaker, smart phone, etc.) can be configured to provide voice personalization for querying or prompting outputs audibly and/or visually output to a patient to try and improve the usability of the device or system and also improve the quality of patient data obtained via patient input provided in response to such prompts or queries. FIG. 15 illustrates an exemplary voice personalization process that can be utilized by the input/output device 13 and/or wearable device 3. The central server 7 and/or docking station 5 can also be configured to utilize such a process for causing audible outputs to a patient to occur via a personalized voice for different prompts and/or queries that may be provided to a patient.

For instance, natural language processing can be utilized to generate voice data for pre-selected prompts and queries to be communicated to a user audibly via a speaker. The wearable device 3, input/output device 13, docking station 5, and/or central server 7 can also be configured to facilitate a replacement of that voice data so that the voice data that is audibly output sounds to be the voice of a particular person known to the patient (e.g. the patient's spouse, child, uncle, aunt, relative, or friend). For instance, the voice of the particular person can be recorded via a microphone and that data can be used to generate the personalized voice data for output. In some embodiments, such a recording can be of the person actually saying the entire prompt or query for each prompt or query. In other embodiments, voice samples of the person can be acquired via microphone for storage and then used to generate voice data prompts that have a sound that mimics the sound of the person's voice based on the stored personalized voice data.

The personalized voice data can also be updated to improve its quality of mimicking the voice of the person. For example, a user interface can be utilized by the patient to trigger further voice data acquisition via a microphone for the collection of additional voice samples for improving the personalized voice data generated by the device.

In some embodiments, the personalized voice data that is collected can be transmitted to central server 7 via input/output device 13, wearable device 3 and/or docking station 5 for storage and use in a voice cloud database. Such a database can link the personalized voice data to the patient so that voice prompts and queries provided via different user interfaces of the wearable device 3, input/output device 13, and/or docking station 5 are provided via personalized voice generated via the stored personalized voice data in the voice cloud database. Such personalized voice data prompts and queries can be utilized in the collection of subjective data from patients as discussed herein (e.g. FIG. 17) in addition to other prompts or queries that can be output to a patient in connection with a user interface of each device of the system.

The collection and storage of the user's objective and subjective data related to the user's sleep, sleep quality, and/or sleep duration can be stored for analysis and evaluation. The stored data can also be utilized to generate displays for displaying to the user the user's experienced sleep patterns for a particular period of time (e.g. weeks, days, months, etc.). Such displays can include graphs, bar charts, or other graphical displays or video displays illustrating such graphs or charts in a video form. The stored data can also be stored for evaluation of different user specific patterns of behavior for use in suggesting changes to that behavior for attempting to improve the user's sleep quality and/or duration. The stored data can also be grouped with the stored data of other users for collecting a larger database of sleep related data to allow for a big data evaluation of this data to improve on behavioral suggestions and/or behavior pattern detection that could be provided by the system for identifying and suggesting behavioral changes to improve a user's sleep duration and/or quality.

A graphical user interface GUI can be caused to be displayed via a display 13a of the wearable device 3, docking station 5, input/output device 13, or a user computer device that can be in communicative connection with the central server 7. The GUI can be configured to provide visual output to the user and allow the user to provide input in response to queries or other prompts that may be provided to the user via the GUI and/or an audible prompt provided by a speaker of the device. Such data can include subjective data that is to be communicated to the central server 7 or stored locally on the device for use by the application defining the program for the GUI as well as other features of the device and its user of sensor data obtained from the wearable device 3. As may be seen from the example of FIG. 28, a user can be shown a display of one or more calculated scores related to the user's health and detected sleep quality. These scores can also be shown to indicate improvement or degradation of a user health metric that may be occurring over time. Additional indicia can be displayed to actuate a display of additional information related to any particular score or health metric as well. In response to the actuation of such indicia, the device can generate another display to provide information determined from the sensor data as well as subjective data input provided by the user.

It should be appreciated that the sleep score calculation as well as other health metric monitoring and score calculations can be utilized to develop multiple different sleep baseline scores for evaluation of a user's health or sleep. For example, after a first baseline sleep score evaluation is made and a first baseline sleep score is determined, a user may utilize a drug or other type of medical intervention to try and improve his or her sleep. Such an improvement (or lack thereof) can be evaluated by comparing the sleep a user may experience while using the medical intervention, which can also be referred to herein as a behavioral change (e.g. drug, meditation, yoga, or other intervention), with the first baseline sleep data and first baseline sleep score that was obtained prior to that intervention.

For example, after the baseline first sleep information is obtained as discussed herein, the wearable device 3, input/output device 13, or central server 7 can receive data indicating the user is undergoing an intervention (e.g. a treatment), such as receiving a first input indicating that the user is taking a newly prescribed medication, changing the frequency or dosage of that medication, changing the user's diet, or changing a user activity (e.g. engaging in meditation or yoga at an increased frequency etc.). In response to that first input concerning the intervention, the wearable device 3 can be used to evaluate a new second baseline sleep score for the user after the user has engaged in the intervention activity. The new second baseline sleep score can be obtained using the same process discussed above with reference to the first sleep score or first baseline sleep score. For example, the user's sleep can be detected and monitored for evaluation after the user engages in the intervention to obtain a new baseline sleep score. The new baseline sleep score can be determined after an intervention timer period has passed after the intervention was initiated. This time period can also be considered a pre-selected sleep improvement detection time period. For instance, the determination of the new baseline can be initiated one day after a new drug is prescribed and taken or after 30 days of the new drug being taken if it may take 30 days before the drug is likely to be effective. The continued effectiveness of the drug may also be monitored by comparing the baseline sleep score to a sleep score evaluated monthly, at two-month, four-month, six-month, eight-month, ten-month or yearly intervals and/or another efficacy evaluation time period. Other new baseline time periods can also be utilized for different interventions to meet the particular time related properties of the particular intervention being utilized for evaluation and comparison of that intervention and its effect on the user.

The new sleep score baseline can be determined by collecting sensor data for the user's sleep and evaluation within a new sleep score baseline time period. This time period can be set for recording sensor data for the new sleep score baseline time period. This time period can be 24 hours, multiple days, or a week, for example. After this time period has elapsed, sensor data collected during the sleep baseline time period can be evaluated. This data can include accelerometer data that includes detected motion data along multiple axes that was collected during sleep, user heart rate (HR), noise of the environment (ENV) recorded during the user's sleep, user's temperature, and other parameters. For instance, the accelerometer data can include accelerometer x direction data Ax, accelerometer y direction data Ay, and accelerometer z direction data Az collected for each time segment of a particular measurement time. The x direction can be horizontal, the y-direction can be vertical (e.g. a direction along an axis that extends vertically upward and downward), and the z direction can be a direction that is perpendicular to vertical and perpendicular to the horizontal x direction. For example, the x direction can be a direction along a horizontally extending axis that extend horizontally in right and left side directions and the z direction can be a direction along a horizontal axis that extends in front and rear directions.

The accelerometer data as well as the other sensor data can be filtered and subsequently used to calculate new baseline sleep score weights and a new baseline sleep score. The filtering that is performed can be to filter out the accelerometer data or other sensor data that does not meet or exceed a pre-determined sleep threshold level of sleep motion or other sleep related data parameter (e.g. noise is not above a pre-selected light sleep and/or a pre-selected deep sleep noise threshold, etc.).

Based on the new baseline sleep data, varying weights can also be determined. The weights can include a first weight w1, a second weight w2, a third weight w3, and a fourth weight w4. The new baseline sleep data and weights can be utilized to calculate the new baseline sleep score. The new sleep score (e.g. a second sleep score for comparison to an older first sleep score or a third sleep score for comparison to an older second sleep score, etc.) can be calculated according to the same aforementioned formula(s) used to form the baseline tremor score. The new sleep score can then be compared with the old baseline sleep score (e.g. a second baseline sleep score can be compared to a first baseline sleep score or a third sleep score can be compared to a second sleep score or first sleep score that was previously calculated at an earlier time) to determine if the intervention has provided an improvement or had no meaningful effect. The evaluation can include, for example, comparing the difference between first and second sleep baseline scores with a pre-selected efficacy value that is defined to help evaluate whether a change of statistical significance has occurred in the user's sleep that were determined from the comparison. If no improvement or meaningful effect is found to have occurred, a change to the intervention can be made (e.g. increase dosage of new drug from a first dosage to a second dosage, adjust frequency of taking the drug from a first frequency to a second frequency (e.g. from once a day to once every 12 hours or once a day to once every six hours, etc.), adjust to a different drug being taken, further adjust user activity or diet, etc.). After the change in dosage of the drug from the first dosage to the second dosage, drug taking frequency (e.g. a change from a first drug frequency to a second drug frequency), drug taken, or other change is made, the second baseline sleep score can be compared to a third baseline sleep score that is obtained using the similar process used to generate the second baseline sleep score after this change has begun (e.g. using the sensors to obtain additional sensor data, etc. for the second change in dosage, frequency, drug, and/or other behavior and processing that data as discussed above). The calculation of the third baseline sleep score can occur in response to receipt of a second input indicating a change has occurred to trigger the calculation or determination of the new baseline sleep score.

The third baseline sleep score can then be compared to the first baseline sleep score and/or the second baseline sleep score to evaluate whether the additional change has a meaningful effect. If no improvement is provided, yet another change can again be made to either drug dosage (e.g. a third dosage can be utilized as another change), frequency (e.g. a third drug frequency can be utilized as another changer), drug taken, or another parameter. This process of drug dosage, frequency or drug adjustment (or other activity adjustment) can be iteratively performed multiple times repeatedly until a meaningful improvement has been obtained (e.g. there may be changes and sensor data obtained from a fourth drug dosage, fourth drug frequency, and fourth baseline tremor score, . . . etc.). The time taken between performing the sleep score baseline calculations can be considered a pre-selected improvement time period (e.g. a time period after the change would be expected to result in a meaningful change in the user's sleep or health). This same type of evaluation process can also be utilized to evaluate a change in medication from one drug to another new drug. Once the change in medication is utilized by the patient, the drug dosage, frequency, and other parameters can be evaluated and changed as discussed herein to evaluate the effectiveness of the new drug as compared to how the patient's health was while using the prior drug.

It should be appreciated that modifications to the embodiments explicitly shown and discussed herein can be made to meet a particular set of design objectives or a particular set of design criteria. For example, the sensor array 3a can utilize more sensors 4 or less sensors 4 than shown in the exemplary embodiment of FIGS. 1-5 and 18-20. As another example, the wearable device 3 can have a housing 3d that is configured to be attached to an adjuster so that the housing has an elliptical annular shape, circular annular shape, or other types of annular shape so that a central opening defined by the housing 3d and/or straps of the housing can receive a portion of a user's body (e.g. neck, arm, leg, wrist, ankle, upper arm, lower arm, upper leg, lower leg, waist, chest, etc.) The size, width, and thickness of such components can be any particular dimension that may help meet a particular set of design criteria. As another example, the type of processor, circuit board, display, or non-transitory computer readable medium of the wearable device can be designed for meeting a particular set of design objectives or a particular design criteria. As yet another example, one or more input/output devices 13 can be connectable to the wearable device to facilitate the collection of subjective data or outputting of information about the collected health data to a user. The input/output devices 13 can include a smart speaker, a smart phone, a tablet, a laptop computer, a personal computer, or other types of personal electronic device having a speaker and/or display for outputting data to the user while also having at least one input device (e.g. a pointer device, a keypad, a button, a microphone, etc.) to receive input data from the user. As yet another example, the type of data encryption and data collection and transmission protocols utilized can be any of a number of suitable options for meeting a particular set of design criteria. As yet another example, the type of input/output device 13 that can be utilized in embodiments of the communication system and embodiments of methods can be a number of different types of devices including without limitation, a smart speaker, a smart phone, an electronic tablet, a personal computer device, a laptop computer device, or a medical condition monitoring device.

As another example, it is contemplated that a particular feature described, either individually or as part of an embodiment, can be combined with other individually described features, or parts of other embodiments. The elements and acts of the various embodiments described herein can therefore be combined to provide further embodiments. Thus, while certain exemplary embodiments of the electronic devices and communication systems that can be configured to facilitate the monitoring of a patient's sleep and/or the efficacy of a sleep treatment for a patient who has been diagnosed as having a sleep related health issue as well as methods of making and using the same and methods of evaluating a sleep treatment and methods of facilitating the diagnosis of a sleep related health issue have been shown and described above, it is to be distinctly understood that the invention is not limited thereto but may be otherwise variously embodied and practiced within the scope of the following claims.

1-129. (canceled)

130. An apparatus for health monitoring comprising:
a wearable device comprising a processor, a non-transitory computer readable medium connected to the processor; and a sensor array connected to the processor and/or the non-transitory computer readable medium; and/or
a server communicatively connectable to the wearable deice to receive sensor data from the wearable device; and/or
an input/output device communicatively connectable to the wearable device to receive sensor data from the wearable device.

131. The apparatus of claim 130, wherein the wearable device is configured to obtain the sensor data via the sensor array when a user wears the wearable device, analyze the sensor data to track a condition of the user, and generate output to help the user improve the condition or maintain the condition,
wherein the apparatus includes the server, the input/output device, and the wearable device, and
wherein the condition is a tremor condition, epilepsy, a sleep condition, an Alzheimer's disease condition, a neurological disorder, a neurodegenerative disease associated with a tremor or for which the tremor is a symptom, multiple sclerosis, stroke, traumatic brain injury, Parkinson's disease, ADHD, dementia, Alzheimer's disease, the condition is a result of use of a medicine, the condition is a result of alcohol abuse, the condition is a withdrawal of a drug, the condition is a thyroid condition, the condition is an overactive thyroid, the condition is a liver condition, the condition is liver failure, the condition is kidney failure, the condition is anxiety or the condition is panic.

132. The apparatus of claim 130, wherein the wearable device, the server, and/or the input/output device is configured to evaluate the sensor data to track a condition of the user and determine a baseline for the condition; and
the wearable device, the server, and/or the input/output device is configured to respond to a first input indicating that a drug at a first dosage is being taken by the user by comparing the baseline for the condition with the sensor data obtained after the receipt of the first input to determine whether the condition has improved.

133. The apparatus of claim 132, wherein the wearable device, the server, and/or the input/output device is configured to respond to a determination that the condition has not improved within a pre-specified improvement time period after receipt of the first input by suggesting a change to the user for adjusting a frequency of taking the drug and/or adjusting a dosage of the drug to a second dosage that differs from the first dosage,
wherein the wearable device, the server, and/or the input/output device is configured to respond to a second input indicating that a change to the drug being taken by the user has occurred by comparing the baseline for the condition with the sensor data obtained after the receipt of the second input to determine whether the condition has improved, and
wherein the wearable device, the server, and/or the input/output device is configured to respond to a determination that the condition has not improved within a pre-specified improvement time period after receipt of the second input, by suggesting a change to the user for adjusting a frequency of taking the drug and/or adjusting a dosage of the drug to a third dosage that differs from the first dosage.

134. The apparatus of claim 130, wherein the wearable device, the server, or the input/output device is configured to evaluate the sensor data to track sleep of the user to determine a baseline level of sleep for the user.

135. The apparatus of claim 134, wherein the wearable device, the server, or the input/output device is configured determine one or more time durations at which the user was in a light sleep state during the sleep, a deep sleep state during the sleep, and/or a rapid eye movement (REM) sleep state during the sleep,
wherein the wearable device, the server, or the input/output device is configured to calculate (i) a total time slept during a monitoring of the sleep, (ii) a time of sleep onset indicating a time it took the user to fall asleep after being detected as attempting to go to sleep, and/or (iii) a sleep efficiency indicating an amount of time the user was asleep during the total time the sleep of the user was monitored, and
wherein the wearable device, the server, or the input/output device is configured to determine (i) an amount of time during the monitored sleep that the user was in the light sleep state, (ii) an amount of time during the monitored sleep that the user was in the deep sleep state, and/or (iii) an amount of time during the monitored sleep that the user was in the REM sleep state.

136. The apparatus of claim 135, wherein the wearable device, the server, or the input/output device is configured to determine a sleep score for the user based on the sensor data obtained from monitoring of the sleep of the user,
wherein the sleep score is determined in accordance with a sleep score formula:
Sleep Score=W_{LS}*LS+W_{DS}*DS+W_{RS}*RS+W_{BMsleep}*BMₛₗₑₑₚ,+W_{ENV}*ENV+W_{Td}*Td+W_{To}*To+W_{Te}*Te;
wherein:
LS is a value for an amount of time the user was determined to be in a light sleep state;
DS is a value for an amount of time the user was determined to be in a deep sleep state;
RS is value for an amount of time the user was determined to be in a REM sleep state;
Td is a value for duration of total sleep time of the user;
To is a value for a determined sleep onset for the sleep;
Te is a value for the determined sleep efficiency for the sleep;
BMₛₗₑₑₚ is a value for the detected body movement of the user during the sleep;
ENV is a value for the detected environment during the sleep;
W_{LS} is the weight for LS;
W_{DS} is the weight for DS;
W_{RS} is the weight for RS;
W_{BMsleep} is the weight for BMₛₗₑₑₚ,
W_{ENV} is the weight for ENV;
W_{Td} is the weight for Td;
W_{To} is the weight for To; and
W_{Te} is the weight for Te.

137. The apparatus of claim 130, wherein the apparatus is configured so that accelerometer data of the sensor data is evaluated for a first tremor baseline time period to determine:
(i) a number of tremors that happened within the first tremor baseline time period to determine a Tremor Count (T_{C});
(ii) a Tremor Duration (T_{D}) as an amount of time elapsed during occurrence and non-occurrence of a detected tremor for each tremor detected within the first tremor baseline time period based on the sensor data;
(iii) a Tremor Amplitude (T_{A}) in the detected tremor within a single period of the tremor for each tremor detected within the first tremor baseline time period; and
(iv) tremor frequency (T_{F}) as a number of tremor occurrences within the first tremor baseline time period,
wherein a first weight w1, a second weight w2, a third weight w3, and a fourth weight w4 are determined to calculate a first baseline tremor score (first T_{S}) according to:
first T_{S}=(w1*T_{F}+w2*T_{A}+w3*T_{D}+w4*T_{C}),

138. The apparatus of claim 137, wherein the apparatus is configured such that, in response to input indicating a drug is taken by the user, the accelerometer data of the sensor data obtained after the drug is taken by the user is evaluated for a second tremor baseline time period to determine:
(i) a number of tremors that happened within the second tremor baseline time period to determine a Tremor Count (T_{C});
(ii) a Tremor Duration (T_{D}) as an amount of time elapsed during occurrence and non-occurrence of a detected tremor for each tremor detected within the second tremor baseline time period based on the sensor data;
(iii) a Tremor Amplitude (T_{A}) in the detected tremor within a single period of the tremor for each tremor detected within the second tremor baseline time period; and
(iv) tremor frequency (T_{F}) as a number of tremor occurrences within the second tremor baseline time period, and
wherein a first weight w1, a second weight w2, a third weight w3, and a fourth weight w4 are determined to calculate a second baseline tremor score (second T_{S}) according to:
second T_{S}=(w1*T_{F}+w2*T_{A}+w3*T_{D}+w4*T_{C}).

139. The apparatus of claim 138, wherein the input/output device, the server, or the wearable device is configured to evaluate the accelerometer data to determine the first baseline tremor score,
wherein the input/output device, the server, or the wearable device is configured to evaluate the accelerometer data to determine the second baseline tremor score,
wherein the input/output device, the server, or the wearable device is configured to compare the second baseline tremor score to the first baseline tremor score to evaluate efficacy of the drug, and
wherein the input/output device, the server, or the wearable device is configured to generate output for suggesting a change to a dose of the drug and/or a frequency at which the drug is to be taken in response to determining that (i) the second baseline tremor score indicates a worse tremor condition as compared to the first baseline tremor score, (ii) the second baseline tremor score indicates a tremor condition that is the same as the first baseline tremor score; (iii) that the second baseline tremor score is higher than the first baseline tremor score, (iv) that the second baseline tremor score is within a pre-selected non-efficacy range of the first baseline tremor score, or (iv) that the second baseline tremor score differs from the first baseline tremor score by no more than a pre-selected efficacy value.

140. The apparatus of any of claim 130 wherein the input/output device, server, and/or wearable device is configured to determine a quality of daytime activity (QODA) score, wherein the QODA score is determined based on a formula of:
QODA=Sleep Score+mind,body and diet (MBD) Score;
QODA=Sleep Score+MBD Score+Activity Score;
QODA=Sleep Score*W_{QODASS}+MBD Score*W_{QODAMBD}; or
QODA=Sleep Score*W_{QODASS}+MBD Score*W_{QODAMBD}+Activity Score*W_{QODAAct}
where:
W_{QODASS} is a QODA weight for the Sleep Score;
W_{QODAMBD} is a weight for the MBD Score; and
W_{QODAAct} is a weight for the Activity Score,
wherein the MBD score is determined from:
MBD score=MB*w1_{MBD}+D*w2_{MBD}
where:
MB is a mind and body score based on subjective input the user provided;
D is a diet score based on dietary information of the user;
w1_{MBD} is a weight for the MB score; and
w2_{MBD} is a weight to weigh the diet score D, and
wherein the Sleep Score is determined from:
Sleep Score=W_{LS}*LS+W_{DS}*DS+W_{RS}*RS+W_{BMsleep}*BMₛₗₑₑₚ,W_{ENV}*ENV+W_{Td}*Td+W_{To}*To+W_{Te}*Te;
wherein:
LS is a value for an amount of time the user was determined to be in a light sleep state;
DS is a value for an amount of time the user was determined to be in a deep sleep state;
RS is value for an amount of time the user was determined to be in a REM sleep state;
Td is a value for duration of total sleep time of the user;
To is a value for a determined sleep onset for the sleep;
Te is a value for the determined sleep efficiency for the sleep;
BMₛₗₑₑₚ is a value for the detected body movement of the user during the sleep;
ENV is a value for the detected environment during the sleep;
W_{LS} is the weight for LS;
W_{DS} is the weight for DS;
W_{RS} is the weight for RS;
W_{BMsleep} is the weight for BMₛₗₑₑₚ,
W_{ENV} is the weight for ENV;
W_{Td} is the weight for Td;
W_{To} is the weight for To; and
W_{Te} is the weight for Te.

141. A method of monitoring a health condition of a user comprising:
obtaining sensor data via a sensor array when a user wears a wearable device having the sensor array;
analyzing the sensor data to track a condition of the user;
generating output to suggest a change to help the user improve the condition or maintain the condition based on the analyzed sensor data.

142. The method of claim 141, wherein the analyzing of the sensor data to track the condition of the user is performed to determine a first baseline for the condition, the method further comprising:
responding to a first input indicating that a drug at a first dosage is being taken by the user by comparing the baseline for the condition with the sensor data obtained after the receipt of the first input to determine whether the condition has improved;
responding to a determination that the condition has not improved within a pre-specified improvement time period after receipt of the first input by suggesting a change to the user for adjusting a frequency of taking the drug and/or adjusting a dosage of the drug to a second dosage that differs from the first dosage and/or adjusting the drug to a different drug indicated for the condition; and
responding to a second input indicating that a change to the drug being taken by the user has occurred by comparing the baseline for the condition with the sensor data obtained after the receipt of the second input to determine whether the condition has improved.

143. The method of claim 142, wherein the analyzing the sensor data to track the condition of the user includes determining a baseline level of sleep for the user, the method further comprising:
monitoring sleep of the user based on the sensor data obtained when the user wears the wearable device having the sensor array while sleeping to determine one or more time durations at which the user was in a light sleep state during the sleep, a deep sleep state during the sleep, and/or a rapid eye movement (REM) sleep state during the sleep;
calculating: (i) a total time slept during a monitoring of the sleep, (ii) a time of sleep onset indicating a time it took the user to fall asleep after being detected as attempting to go to sleep, and/or (iii) a sleep efficiency indicating an amount of time the user was asleep during the total time the sleep of the user was monitored; and
determining (i) an amount of time during the monitored sleep that the user was in the light sleep state based on the sensor data, (ii) an amount of time during the monitored sleep that the user was in the deep sleep state based on the sensor data, and/or (iii) an amount of time during the monitored sleep that the user was in the REM sleep state based on the sensor data.

144. The method of claim 143, further comprising:
determining a sleep score for the user based on the sensor data obtained from monitoring of the sleep of the user,
wherein the sleep score is determined in accordance with a sleep score formula:
Sleep Score=W_{LS}*LS+W_{DS}*DS+W_{RS}*RS+W_{BMsleep}*BMₛₗₑₑₚ,+W_{ENV}*ENV+W_{Td}*Td+W_{To}*To+W_{Te}*Te;
wherein:
LS is a value for an amount of time the user was determined to be in a light sleep state;
DS is a value for an amount of time the user was determined to be in a deep sleep state;
RS is value for an amount of time the user was determined to be in a REM sleep state;
Td is a value for duration of total sleep time of the user;
To is a value for a determined sleep onset for the sleep;
Te is a value for the determined sleep efficiency for the sleep;
BMₛₗₑₑₚ is a value for the detected body movement of the user during the sleep;
ENV is a value for the detected environment during the sleep;
W_{LS} is the weight for LS;
W_{DS} is the weight for DS;
W_{RS} is the weight for RS;
W_{BMsleep} is the weight for BMₛₗₑₑₚ,
W_{ENV} is the weight for ENV;
W_{Td} is the weight for Td;
W_{To} is the weight for To; and
W_{Te} is the weight for Te.

145. The method of claim 141, further comprising:
evaluating accelerometer data of the sensor data for a first tremor baseline time period to determine:
(i) a number of tremors that happened within the first tremor baseline time period to determine a Tremor Count (T_{C});
(ii) a Tremor Duration (T_{D}) as an amount of time elapsed during occurrence and non-occurrence of a detected tremor for each tremor detected within the first tremor baseline time period based on the sensor data;
(iii) a Tremor Amplitude (T_{A}) in the detected tremor within a single period of the tremor for each tremor detected within the first tremor baseline time period; and
(iv) tremor frequency (T_{F}) as a number of tremor occurrences within the first tremor baseline time period; and
determining a first weight w1, a second weight w2, a third weight w3, and a fourth weight w4 to calculate a first baseline tremor score (first T_{S}) according to:
first T_{S}=(w1*T_{F}+w2*T_{A}+w3*T_{D}+w4*T_{C}).

146. The method of claim 145, further comprising:
in response to input indicating a drug is taken by the user, the accelerometer data of the sensor data obtained after the drug is taken by the user is evaluated for a second tremor baseline time period to determine:
(i) a number of tremors that happened within the second tremor baseline time period to determine a Tremor Count (T_{C});
(ii) a Tremor Duration (T_{D}) as an amount of time elapsed during occurrence and non-occurrence of a detected tremor for each tremor detected within the second tremor baseline time period based on the sensor data;
(iii) a Tremor Amplitude (T_{A}) in the detected tremor within a single period of the tremor for each tremor detected within the second tremor baseline time period; and
(iv) tremor frequency (T_{F}) as a number of tremor occurrences within the second tremor baseline time period;
determining a first weight w1, a second weight w2, a third weight w3, and a fourth weight w4 a to calculate a second baseline tremor score (second T_{S}) according to:
second T_{S}=(w1*T_{F}+w2*T_{A}+w3*T_{D}+w4*T_{C}); and
comparing the second baseline tremor score to the first baseline tremor score to evaluate efficacy of the drug.

147. The method of claim 146, further comprising:
changing a dose of the drug and/or a frequency at which the drug is to be taken in response to determining that (i) the second baseline tremor score indicates a worse tremor condition as compared to the first baseline tremor score, (ii) the second baseline tremor score indicates a tremor condition that is the same as the first baseline tremor score; (iii) that the second baseline tremor score is higher than the first baseline tremor score, (iv) that the second baseline tremor score is within a pre-selected non-efficacy range of the first baseline tremor score, or (iv) that the second baseline tremor score differs from the first baseline tremor score by no more than a pre-selected efficacy value.

148. The method of claim 141, comprising:
determining a quality of daytime activity (QODA) score based on the sensor data, wherein the QODA score is determined based on a formula of:
QODA=Sleep Score+mind,body and diet (MBD) Score;
QODA=Sleep Score+MBD Score+Activity Score;
QODA=Sleep Score*W_{QODASS}+MBD Score*W_{QODAMBD}; or
QODA=Sleep Score*W_{QODASS}+MBD Score*W_{QODAMBD}+Activity Score*W_{QODAAct}
where:
W_{QODASS} is a QODA weight for the Sleep Score;
W_{QODAMBD} is a weight for the MBD Score; and
W_{QODAAct} is a weight for the Activity Score,
wherein the MBD score is determined from:
MBD score=MB*w1_{MBD}+D*w2_{MBD}
where:
MB is a mind and body score based on subjective input the user provided;
D is a diet score based on dietary information of the user;
w1_{MBD} is a weight for the MB score;
w2_{MBD} is a weight to weigh the diet score D, and
wherein the Sleep Score is determined from:
Sleep Score=W_{LS}*LS+W_{DS}*DS+W_{RS}*RS+W_{BMsleep}*BMₛₗₑₑₚ, +W_{ENV}*ENV+W_{Td}*Td+W_{To}*To+W_{Te}*Te;
wherein:
LS is a value for an amount of time the user was determined to be in a light sleep state;
DS is a value for an amount of time the user was determined to be in a deep sleep state;
RS is value for an amount of time the user was determined to be in a REM sleep state;
Td is a value for duration of total sleep time of the user;
To is a value for a determined sleep onset for the sleep;
Te is a value for the determined sleep efficiency for the sleep;
BMₛₗₑₑₚ is a value for the detected body movement of the user during the sleep;
ENV is a value for the detected environment during the sleep;
W_{LS} is the weight for LS;
W_{DS} is the weight for DS;
W_{RS} is the weight for RS;
W_{BMsleep} is the weight for BMₛₗₑₑₚ,
W_{ENV} is the weight for ENV;
W_{Td} is the weight for Td;
W_{To} is the weight for To; and
W_{Te} is the weight for Te.

149. A wearable device comprising:
a processor;
a non-transitory computer readable medium connected to the processor;
a sensor array connected to the processor and/or the non-transitory computer readable medium,
wherein the sensor array comprises:
an optical sensor to monitor heart rate and blood oxygen content;
a temperature sensor to measure temperature of a user wearing the wearable device;
a microphone to detect audible noise during sleep;
a sweat sensor to measure sweat of the user, and
wherein the wearable device is configured to periodically evaluate sensor data to detect a heart rate, body movement and sweat of a user wearing the wearable device and, upon a determination that the heart rate, body movement, and sweat exceed a pre-selected threshold sleep condition criteria, cause at least one output to be emitted to improve a duration and/or quality of sleep of the user,
wherein the output includes the wearable device vibrating via a vibration mechanism and/or triggering an audible output of at least one sound or music.
